(19) United States
(12) Patent Application Publication
(10) Pub. No.: US 2007/0243132 A1
(43) Pub. Date: Oct. 18, 2007

(54) Transdermal delivery of pharmaceutical agents

(75) Inventors: Gregory J. Russell-Jones, Middlecove (AU); Michael R. Luke, Potts Point (AU); Stewart R. Himes, Dee Why (AU)

Correspondence Address: SEED INTELLECTUAL PROPERTY LAW GROUP PLLC, 701 FIFTH AVE, SUITE 5400, SEATTLE, WA, 98104, US

(73) Assignee: Apollo Life Sciences Limited, Beaconsfield (AU)

(21) Appl. No.: 11/645,122

(22) Filed: Dec. 22, 2006

Related U.S. Application Data
(60) Provisional application No. 60/753,454, filed on Dec. 22, 2005.

(30) Foreign Application Priority Data
Sep. 15, 2006 (AU) 2006905107

(51) Int. Cl. A61K 51/12; A61K 31/56; A61K 31/60; A61K 31/7052; A61K 35/32; A61K 35/36; A61K 38/16; A61K 38/18; A61K 38/19; A61K 38/20; A61K 38/21; A61K 38/22; A61K 38/28; A61K 38/43; A61K 39/00; A61K 39/395

(52) U.S. Cl. 424/1.11; 424/130.1; 424/139.1; 424/184.1; 424/548; 424/572; 424/718; 424/85.1; 424/85.2; 424/85.4; 424/94.1; 514/1; 514/12; 514/154; 514/159; 514/169; 514/3; 514/44; 514/458; 514/474; 514/52

(57) ABSTRACT

The present invention generally relates to a vehicle useful for delivering a pharmaceutically active compound including a genetic molecule or composition. More particularly, the present invention provides microemulsions for transdermal delivery of pharmaceutically active agents to a subject.

### CROSS-REFERENCE TO RELATED APPLICATIONS

This application claims the benefit under 35 U.S.C. § 119(e) of U.S. Provisional Patent Application No. 60/753,454 filed Dec. 22, 2005; and Australian Provisional Patent Application No. 2006905107 filed Sep. 15, 2006; where these (two) provisional applications are incorporated herein by reference in their entireties.

### BACKGROUND OF THE INVENTION

1. Field of the Invention

The present invention generally relates to a vehicle useful for delivering a pharmaceutically active compound including a genetic molecule or composition transdermally to a subject. More particularly, the present invention provides microemulsions for transdermal delivery of pharmaceutically active agents to a subject.

2. Description of the Prior Art

Bibliographic details of references provided in the subject specification are listed at the end of the specification.

Reference to any prior art in this specification is not, and should not be taken as, an acknowledgment or any form of suggestion that this prior art forms part of the common general knowledge in any country.

It is generally accepted in the art that delivery of therapeutic quantities of water-soluble macromolecules such as peptides and proteins or other such pharmaceutical agents across the skin is extremely difficult, as the skin functions as a barrier to prevent transdermal penetration of most water-soluble molecules.

However, transdermal delivery is a highly desired method by which therapeutic amounts of peptides, proteins or other pharmaceutical agents, could be administered particularly for local and/or systemic therapy. This is because biologically-active macromolecules, such as certain peptides or proteins, generally have low oral bioavailability, making oral administration difficult, and often have short biological half-lives, making parenteral delivery impractical outside a hospital setting (Shahrokh et al. Therapeutic Protein and Peptide Formulation and Delivery, American Chemical Society, 1997). Additionally, there are many medical conditions and diseases of the skin, which would greatly benefit from direct local therapy, without exposing the other organs of the body to the deleterious side-effects of delivery via other routes. In this regard various pharmaceutical agents, such as non-steroidal anti-inflammatory drugs (NSAIDs), have been shown to have considerable gastric toxicity, thereby reducing their effectiveness. Additional side effects of NSAIDs include renal insufficiency and failure, gastrointestinal ulceration, bleeding or perforation, exacerbation of hypertension and congestive heart failure.

Delivery of therapeutic pharmaceutical agents across the skin addresses the above-mentioned problems by offering a number of potential advantages compared to conventional methods, such as pills and injections, including (1) little or no degradation or modification of the pharmaceutical agents such as digestion of the proteins by the enzymes and surfactants resident in the stomach and small intestine, (2) an increase in drug effectiveness as the transdermal route avoids first pass metabolism by the liver; (3) minimization of gastrointestinal and other side effects caused by ingestion of the pharmaceutical agent; (4) an improvement in patient comfort and compliance due to a more user-friendly delivery method; and (5) the potential for prolonged and controlled drug delivery.

Current research into transdermal transport of water-soluble molecules focuses on (1) using chemical enhancers to alter the skin's lipid environment; (2) using liposomes to facilitate transdermal transport; (3) using iontophoresis to provide an electrical driving force for transdermal transport; (4) using electroporation to create a new transdermal pathway; and (5) using ultrasound to create a new transdermal pathway. Although chemical enhancers have been shown to increase transdermal transport of small compounds, they are often shown to cause significant skin irritation and may affect drug stability (Santus et al. J Control Release 25:1-20, 1993). Introduction of electrical or ultrasonic energy into skin alters its properties as a consequence of the high energy input and issues of safety and drug stability via this method need to be further investigated.

Emulsions have been proposed as a means to enhance transdermal uptake of a small number of hydrophobic molecules. Generally, an emulsion is a composition comprising more than one phase where at least one of the phases consists of a finely divided phase domain (such as, for example, particles or droplets) distributed throughout a continuous phase domain. The finely divided domains are generally referred to as dispersed or discontinuous phase domains. A microemulsion generally is a system comprising an oil phase, a water phase and a surfactant. Some microemulsions are quaternary systems and further comprise a co-surfactant or a co-solvent. Microemulsions differ from ordinary emulsions (or macroemulsions) in that they are thermodynamically stable and form spontaneously upon mixing. The average drop size of ordinary emulsions grows continuously with time so that phase separation ultimately occurs under gravitational force, i.e. they are thermodynamically unstable and their formation requires input of work. In addition, the drops of the dispersed phase are generally large (>0.2 μm) so that ordinary emulsions often take on a milky opaque appearance. In contrast, microemulsions have an observable transparency, which is due to the fact that the maximum size of the droplets of the dispersed phase is not larger than one-half of the wavelength of visible light. On average, the droplet diameter in stable microemulsions is usually within the range of 5-200 nm. Microemulsions also possess specific physicochemical properties such as optical isotropy, low viscosity and thermodynamic stability.

Sintov et al. (Journal of Controlled Release 95:173-183, 2004) describe a system with formulations that contain isopropyl palmitate, water, tetraglycol (as the co-surfactant) and a combination of glyceryl oleate (HLB=2.8) and PEG-40 hydrogenated castor oil (HLB=ca. 15) at a ratio of 1:2 This microemulsion system was loaded with 2.5% lidocaine (a highly lipophilic molecule of low molecular weight) as base or as a hydrochloride salt.

In addition, Kreilgaard et al. (Journal of Controlled Release 69:421-433, 2000) compared microemulsions (i) Labrasol (a mixture consisting of 30% mono-, di and triglycerides of C and C fatty acids, 50% of the ap- mono- and di-esters of poly(ethylene glycol) (PEG 400) and 20% of free PEG 400); (ii) Plurol Isostearique (isostearic acid ester of polyglycerol, containing 30-35% of diglycerol, 20-25% of triglycerol, 15-20% tetraglycerol, and 10% of pentaglycerol and higher oligomers); (iii) isostearylic isostearate (92% purity); and (iv) water on their transdermal delivery potential of a lipophilic (lidocaine) and a low molecular weight hydrophilic model drug (prilocaine hydrochloride). The microemulsions were applied to excised rat skin in a Franz-type diffusion cell and were found to increase transdermal flux of lidocaine through the excised rat skin by up to four times compared to a conventional oil-in-water emulsion, and that of prilocaine hydrochloride by almost 10 times compared to a hydrogel.

Li et al. (International Journal of Pharmaceutics 237:77-85, 2002) examined the use of a microemulsion for intranasal delivery of the hydrophobic drug diazepam. Diazepam, a practically water-insoluble drug, displayed a high solubility of 41 mg/ml in a microemulsion consisting of 15% ethyl laurate, 15% H₂O, and 70% (w/w) surfactant/co-surfactant (Tween 80:propylene glycolethanol at 1:1:1 weight ratio). The study demonstrated an intranasal bioavailability of 50%.

In a study reported by Valenta et al. (Journal of Controlled Release 95:257-265, 2004), the ability of three different microemulsions (A: 2.5 g Brij 97, 7.2 g water and 0.34 g tributyrin; B: 1.87 Brij 97, 5.17 g water and 0.45 miglyol; and C: 2.5 g Brij 97, 6.62 g water and 0.78 g soybean oil) to increase the permeability of the hydrophobic compound sodium fluorescein through porcine skin was examined.

Escribano et al. (European Journal of Pharmaceutical Sciences 19:203-210, 2003) studied the use of microemulsion formed with 19% Transcutanol, 19.5% Plurol oleique, 30.6% water, 10.9% Isostearol isostearate, Labrasol 19% for transdermal delivery of the low molecular weight chemical diclofenac across human skin. The microemulsion was found to be less efficient than a preparation made with the absorption enhancers oleic acid and d-limonene.

However, these microemulsions have not been demonstrated to carry macromolecules, such as certain peptides or proteins, water soluble vitamins, NSAIDs and chemical compounds across the dermis. There is a need, therefore, to develop formulations for transdermal delivery of these molecules.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The present invention provides a topical delivery system which assists penetration of a desired pharmaceutically active agent, such as peptides, polypeptides and proteins, water soluble vitamins, NSAIDs, genetic molecules and chemical compounds into the skin, which does not necessitate the use of chemical enhancers, nor electrical or ultrasonic energy to facilitate penetration into and though the skin. Furthermore, the delivery system is easy to apply to small or large areas of the skin.

The present invention further enables the use of hydrophilic molecules which are incorporated within the submicron sized water droplets contained within the microemulsion, thereby enhancing transdermal delivery of desired pharmaceutical agents.

The topical delivery system of the present invention is in the form of a microemulsion. The subject microemulsions for transdermal delivery of peptides, polypeptides and proteins or other molecules such as water soluble vitamins, NSAIDs, genetic molecules and chemical compounds are able to be formed with little or no shear force applied to the molecules during the preparation of the microemulsion, which aids in the preservation of the structure and bioactivity of these molecules. The microemulsion also provides both a hydrophilic and hydrophobic environment, which can aid in the solubilization of various molecules and which additionally provides the ability to mix hydrophobic molecules and hydrophilic molecules within the same formulation.

Hence, the present invention provides a microemulsion which is capable of transdermal delivery of pharmaceutically active agents. The present invention further extends to a pharmaceutical composition comprising the microemulsion and a pharmaceutically active agent wherein the pharmaceutical composition is suitable for topical application on a subject.

The present invention further provides a method suitable for the transdermal delivery of pharmaceutical agents in a range of therapeutic, cosmetic and research applications. The method comprises (a) preparing a water phase, including dissolving one or more selected pharmaceutically active agents in water; (b) preparing an oil phase, including dissolving one or more selected pharmaceutical agents in the oil phase; (c) emulsifying the water phase and the oil phase thereby forming a topical pharmaceutical composition; and (d) applying the topical pharmaceutical composition on a subject.

The microemulsion of the present invention is useful inter alia for the topical delivery of anti-inflammatory agents, such as small molecules, proteins, antibodies, Fc fusion molecules and soluble receptors; vaccines; imaging agents; anti-cancer agents, particularly anti-melanoma agents; skin lightening including whitening agents; UV blocking agents, agents to reduce or nullify nitric oxide, agents useful for the treatment of erectile dysfunction; agents for increasing local circulation, agents for the treatment of obesity, re-pigmentation agents; genetic molecules such as RNAi, DNA, virosomes and anti-sense RNA; and agents useful in treating muscle dystrophy. The microemulsions have application in human and veterinary medicine as well as for agricultural animals.

A list of agents contemplated for use in the microemulsions includes but is not limited to the list provided in Table 1.

TABLE 1
Agents

| Agents | Description and/or Alternate names/or alternative drugs |
|---|---|
| AdeCbl | Adenosylcobalamin, Cobamide coenzyme, |
| | Deoxyadenosylcobalamin, Cobamamide, Vitamin B12 |
| | coenzyme, Cobamamid, 5,6-Dimethylbenzimidazolyl-5- |
| | deoxyadenosyl-cobamide, Cobinamide, Co-(5′- |
| | deoxyadenosine-5′) derivative hydroxide, dihydrogen |
| | phosphate (ester), inner salt, 3′-ester with 5,6-dimethyl-1-a-D- |
| | ribofuranosyl-1H-benzimidazole, (5′-Deoxy-5′-adenosyl) |
| | cobamide coenzyme, (5,6-dimethylbenzimidazolyl) cobamide |
| | coenzyme, a-(5,6-Dimethylbenzimidazolyl) cobamide |
| | coenzyme, 5′-Deoxy-5′-adenosylcobalamin, 5′- |
| | Deoxyadenosyl vitamin B12, 5′-Deoxyadenosyl-5,6- |
| | dimethylbenzimidazolylcobamide, 5′- |
| | Deoxyadenosylcobalamin, 5,6-Dimethylbenzimidazolyl-Co-5′- |
| | deoxyadenosylcobamide, Calomide, Cobalamin coenzyme, |
| | Cobalamin, Co-(5′-deoxy-5′-adenosyl)-, Cobamide coenzyme, |
| | (5′-deoxy-5′-adenosyl)-, Cobamide coenzyme, (5,6-dimethyl- |
| | 1H-benzimidazolyl)-, Cobinamide, Co-(5′-deoxyadenosine-5′) |
| | derivative, Cobinamide, Co-(5′-deoxyadenosine-5′) derivative., |
| | hydroxide, 3′-ester with 5,6-dimethyl-1-a-D- |
| | ribofuranosylbenzimidazole, DBC coenzyme, Dibencozide, |
| | Funacomide, Vitamin B12, Co-(5′-deoxy-5′-adenosyl) |
| | derivative |
| Amphiregulin | Colorectum cell-derived growth factor (CRDGF); colorectal cell |
| | growth factor (CRGF); colorectal cell-derived growth factor; |
| | colorectum-associated factor; colorectal-associated factor; |
| | keratinocyte-derived autocrine factor (KAF); schwannoma- |
| | derived growth factor (SDGF); AREG |
| Antibacterial agents | Curcumin, tetrahydrocurcumin, sodium circuminate, |
| | bisdemethoxycurcumin, demethoxycurcumin, 5′- |
| | methoxycurcumin and dihydrocurcumin, vitamin E, selenium, |
| | zinc, vitamin C, azaleic acid, pantothenic acid, benzyl peroxide |
| | and extract of tea tree oil, gentamycin, amikacin, neomycin, |
| | penicillin, streptomycin |
| Anti-coagulants | Heparin, Warfarin, Herbal extracts such as those derived from |
| | Danshen, Devil's Claw, Eleuthero, Garlic, Ginger, Ginkgo, |
| | Horse Chestnut, Panax Ginseng, Papain, Red Clover, Saw |
| | Palmetto, capsaicin |
| Antibodies | Antibodies to inflammatory cytokines such as G-CSF, M-CSF |
| | and GM-CSF; anti-TNF alpha antibodies such as infliximab |
| | and adalimumab; antibodies directed against interleukins |
| | including anti IL-1, IL-8, IL-6, IL-12 |
| Anti-cancer agents | Curcumin derivatives, Glycyrrhizin, Glycyrrhetinic acid, |
| | antracycline, platinum drugs, methotrexate derivatives, heary |
| | metals, genistein, chlorambucil, cyclophosphamide, |
| | melphalan, cyclopropane, doxorubicin, daunomycin, |
| | adriamycin, mitomycin C, [2-(hydroxymethyl)anthraquinone], |
| | methotrexate, dichloromethatrexate: cisplatin, carboplatin, |
| | metallopeptides containing platinum, copper, vanadium, iron, |
| | cobalt, gold, cadmium, zinc and nickel, DON, thymidine, |
| | pentamethylmelamin, dianhydrogalactitol, 5-Methyl-THF, |
| | anguidine, maytansine, neocarzinostatin, chlorozotocin, AZQ, |
| | 2′deoxycoformycin, PALA, AD-32, m-AMSA and misonidazole, |
| | doxorubicin, epirubicin and daunorubicin, vincristine, |
| | vinblastin, paclitaxol, taxol, camptothecin, 5-fluorouracil, |
| | leucovorin, Capecitabine, Gemcitabine, Topotecan, 9- |
| | aminocamtotecin, Rubitecan, Irinotecan, Exatecan, Docetaxol, |
| | Doxifluridine, Carmofur, UFT, Eniluracil, ZD-9331, MMI-166, |
| | Eniluracil, 6-hydroxymethylacylfulvene, Nediplatin, Satroplatin, |
| | Lipoplatin, BBR3464, ZD0473, lobaplatin, Spirogermanium, |
| | Gallium trinitrate, etoposide, gallium chloride, Budotitane, |
| | titanocene dichloride, trans-indazolium-[tetrachlorobis(2H- |
| | indazole)ruthenate(III, N1)] (HInd[RuInd2CI4]), Trans- |
| | Na[RuInd2CI4], ruthenium dimethylsulfoxide, thioguanidine, |
| | hexamethylmelamine), 5_nor-anhydro-vinblastine |
| | (Vinorelbine), Avastin (bevacizumab), BEC2 (mitomomab), |
| | Tositumomab (Bexxar), Campath (alemtuzumab), CeaVab, |
| | herceptin (trastuzumab), IMC-C225 (centuximab), Lymphocide |
| | (epratuzumab), MDX-210, Mylotarg (Gemtuzumab), Panorex |
| | (Edorcolomab), Rituxan (Rituximab), Theragyn |
| | (pemtumomab), Zamyl, Zevalin (Ibritumomab), Gefitinib |
| | (Iressa),, Erlotinib, ABX-EGF, GSK 572016 |
| | (lapatinibditosylate), CI-1033 (canertinib diHCl), Bortezomib, |
| | Arsenic trioxide (Trisenox), CC-394 (Actimid), CDC-501 |
| | (Revlimid), Thalidomide (Thalomid), Oblimersen (Genasense), |
| | Abarelix (Plenaxis), Zevalin, Gemtuzumab, 2-MPPA, CPI- |
| | 0004Na |
| Anti-inflammatory | Vitamin B12 derivatives, curcumin derivatives, quercetin, |
| agents | glabridin, phytosphingosine germacrone, turmerone, ar-(+)-, a-, |
| | β-turmerones; β-bisabolene; a-curcumene; zingiberene; β- |
| | sesquiphellandene, bisacurone; curcumenone; |
| | dehydrocurdione; procurcumadiol; bis-acumol; curcumenol; |
| | isoprocurcumenol epiprocurcumenol; procurcumenol; |
| | zedoaronediol; curlone; and turmeronol A and turmeronol B, |
| | NSAIDs include salicylates (including aspirin, choline |
| | magnesium trisalicylate, dilfunisal, salasalate, benorylate); |
| | phenylalkanoic acids (including carpofen, fenoprofen, |
| | fluribiprofen, ibuprofen, ketoprofen, naproxen, naproxen Na, |
| | suprofen, oxaprozin,); acetic acids or indoles (including |
| | alclofenac, diclofenac, fenclofenac, indomethacin, sulindac, |
| | tolemetin); enolic acids (including isoxicam, meloxicam, |
| | piroxicam, tenoxicam); fenamic acids (including flufenamic |
| | acid, meclofenamate, mefenamic acid); napthylalkanones |
| | (including nabumetone); niflumic acid; pyranocarboxylic acids |
| | (including etodolan); pyrazolones (including phenylbutazone, |
| | oxyphenobutazone); pyrroles (including ketorolac); COX-2 |
| | inhibitors (including celecoxib, parecoxib, valdecoxib, |
| | rofecoxib) |
| Anti-oxidants | Curcumin derivatives, ellagic acid, bixin, alpha-tocopherol, |
| | vitamin C, Vitamin E, quinidine, silibinin and silymarin, |
| | germacrone, turmerone, ar-(+)-, a-, β-turmerones; β- |
| | bisabolene; a-curcumene; zingiberene; β-sesquiphellandene, |
| | bisacurone; curcumenone; dehydrocurdione; procurcumadiol; |
| | bis-acumol; curcumenol; isoprocurcumenol epiprocurcumenol; |
| | procurcumenol; zedoaronediol; curlone; and turmeronol A and |
| | turmeronol B |
| Antiplatelet factors | Plavix, Ticlid |
| Anti-TNF molecules | Humira, Remicade, Enbrel, ALSTII |
| Anti-venoms | |
| BAFF | B-cell-activating factor; TNF-and APO L-related leukocyte |
| | expressed ligand 1; TNF and ApoL related leukocyte |
| | expressed ligand-1 (TALL-1, TALL1); B lymphocyte stimulator |
| | (BlyS); B cell-activating factor; dendritic cell-derived TNF-like |
| | molecule; UNQ401/PRO738; TNF homologue activating |
| | apoptosis; nuclear factor-kappaB and c-Jun NH2-terminal |
| | kinase (THANK); ZTNF4; tumor necrosis factor ligand |
| | superfamily member 13B (TNFSF13B) |
| BDNF | Brain-derived neurotrophic factor |
| bFGF | Basic fibroblast growth factor, FGF2 |
| Blood Glucose | Actos, Amaryl, Avandia, Glipizide (Glucotrol XL), glybuzide |
| modifying drugs | (Glynase), Glyset, Metofrmin (Glucophase), Prandin, Precose, |
| | herbal extracts such as those derived from Eleuthero, |
| | Fenugreek, Ginger, Kudzu, Panax Ginseng |
| BMP | Bone morphogenetic protein |
| BMP-4 | Bone morphogenetic protein 4 (BMP4); BMP2B; BMP2B1; |
| | ZYME; decapentaplegic-Vg-related 4 (DVR4) |
| BMP-7 | Bone morphogenic protein 7 (BMP7); osteogenic protein 1 |
| | (OP1, OP-1) |
| Breast Cancer | 5-FU, anthracylines, doxorubicin, epirubicin, idarubycin, and |
| treatment | daunorubicin Platinum drugs, Cisplatin, oxaliplatin, |
| | carboplatin, diaminocyclohexylplatinin (DACH-platinum), |
| | nedaplatin, cycloplatan, SKI2053R, Loboplatin, enloplatin, |
| | zeniplatin, Miboplatin, L-NDDP, TRK-710, MBA (bis |
| | monobromo acetato trans 1,2-diaminocyclohexane platinum |
| | II), PYP (Bis-pyruvato 1,2-diaminocyclohexaneplatinum II), |
| | Sulphato 1,2 diaminocyclohexane platinum II, Malonato 1,2 |
| | diaminocyclohexane platinum II, Isocitrato 1,2- |
| | diaminocyclohexane platinum II, 4-Carboxyphtalato 1,2- |
| | diaminocyclohexane platinum II, bis-monobromoacetato trans |
| | 1,2-diaminocyclooctane platinum II, 1,1 diaminomethyl |
| | cyclohexane sulphate platinum II, JM-216 (bis-acetato- |
| | ammine-dichloro-cyclohexylamine platinum IV, Iproplatin (cis- |
| | dichloro-trans-dihydroxy-bis-isopropylamino platinum IV, |
| | CHIP, JM-9), Iproplatin (cis-dichloro-trans-dihydroxy-bis- |
| | isopropylamino platinum IV, CHIP, JM-9) genistein, curcumin |
| | derivatives, tamoxifen, ¹³¹1, ⁶⁷Cu, ²¹¹At, ²¹²Bi, and IsS/IseRe |
| BSA | Bovine Serum Albumin |
| CD209L | Liver/lymph node specific ICAM-3-grabbing non-integrin (L- |
| | SIGN); dendritic cell specific ICAM-3 grabbing non-integrin |
| | related (DCSIGNR) molecule; DC-SIGN2; CD209L1; |
| | HP10347 |
| Cholesterol Lowering | Curcumin extracts, demethoxycurcumin and |
| drugs | bisdemethoxycurcumin |
| COX | Cyclooxygenase |
| Cox inhibitors | |
| CSF | Colony Stimulating Factor |
| Curcuminoid | Curcumin, tetrahydrocurcumin, sodium circuminate, |
| derivatives | bisdemethoxycurcumin, demethoxycurcumin, 5′- |
| | methoxycurcumin and dihydrocurcumin, |
| | tetrahydrodemethoxycurcumin (INCI: |
| | tetrahydrodemethoxydiferuloymethane), and |
| | tetrahydrobisdemethoxycurcumin (INCI: |
| | tetrahydrobisdemethoxydiferuloylmethane |
| EGF | Epidermal Growth Factor |
| EPO | Erythropoietin; Epoetin alpha; Epogen; Epoetin delta; Dynepo |
| Fas-Ligard | FasL; apoptosis antigen ligand; apoptosis antigen ligand 1 |
| | (APTL, APO-1, APT1LG1); CD178; CD95 ligand (CD95L); |
| | FASLG |
| Fc fragments | Fragment Crystallizable or Immunoglobulin constant region |
| FGF | Fibroblast growth factor |
| FGF2 | Basic Fibroblast Growth Factor, bFGF |
| FGFR1 | Fibroblast growth factor receptor 1 (FRFR-1); basic fibroblast |
| | growth factor receptor (bFGF-R); BFGFR; Fms-like tyrosine |
| | kinase-2 (Pfeiffer syndrome); Fms-related tyrosine kinase 2; |
| | FLT2; heparin binding growth factor receptor; FLG; c-FRG; |
| | CEK; KAL2; N-SAM; N-SAM tyrosine kinase; hydroxyaryl |
| | protein kinase; protein tyrosine kinase; tyrosylprotein kinase |
| FGFR4 | Fibroblast growth factor receptor 4 (FGFR-4); EC 2.7.1.112; |
| | JTK2, MGC20292; TKF; hydroxyaryl-protein kinase; protein- |
| | tyrosine kinase; tyrosine kinase related to fibroblast growth |
| | factor receptor; tyrosylprotein kinase |
| Flt3 | Fms Like Tyrosine kinase 3; Flt3R; EC 2.7.1.112; stem cell |
| | tyrosine kinase 1; STK-1; CD135 antigen; FLK2 |
| Flt3-Ligand | Fms Like Tyrosine kinase 3 ligand; Flt3 ligand (Flt3L); SL |
| | cytokine; Fms-related tyrosine kinase 3 ligand; FLT3LG; FLEX |
| G-CSF | Granulocyte colony stimulating factor (GCSF); colony |
| | stimulating factor 3 (granulocyte) (CSF3); colony stimulating |
| | factor-2; colony stimulating factor-beta; 5637-derived factor; |
| | DF differentiation factor; leukemia cell differentiation inducing |
| | factor; G-CSA granulocyte neutrophil colony stimulating |
| | activity; GM-DF granulocyte/macrophage and leukemia cell |
| | differentiation inducing factor; LBGF leukemic blast growth |
| | factor; M1 differentiation inducing activity; MGI-1G |
| | macrophage-granulocyte inducer-1G; MGI-2 macrophage- |
| | granulocyte inducer-2; NAP-IF neutrophil alkaline |
| | phosphatase inducing factor; Pluripoietin; Pluripoietin-beta; |
| | pCSF pluripotent colony stimulating factor; SCIF suppressor |
| | cell inducing factor |
| GM-CSF | Granulocyte-macrophage colony stimulating factor (GMCSF); |
| | colony stimulating factor 2 (granulocyte-macrophage) (CSF2) |
| | (approved gene symbol); burst promoting activity (BPA); |
| | colony stimulating factor-alpha or beta (CSF-alpha or CSF- |
| | beta); colony stimulating factor 2 (CSF-2); eosinophil colony |
| | stimulating factor (Eo-CSF); eosinophil viability enhancing |
| | factor; eosinophil stimulation promoter (ESP); granulocyte |
| | macrophage colony stimulating activity (GM-CSA); |
| | hematopoietic cell growth factor (HCGF); histamine-producing |
| | cell stimulating factor (HCSF); KM102 burst promoting activity |
| | (KM102-BPA); keratinocyte-derived T-cell growth factor |
| | (KTGF); leukemic blast growth factor (LBGF); macrophage |
| | fusion factor (MFF); macrophage granulocyte inducer (MGI |
| | 1GM); neutrophil migration inhibition factor from T- |
| | lymphocytes or T-cell derived neutrophil migration inhibition |
| | factor (NIF-T); pluripoietin alpha; WEHI-3B differentiation |
| | inducing factor |
| H-2 Receptor | Cimetidine (Tagemet), famotidine (pepcid), nizatidine (Axid), |
| blockers | ranitidine (Zantac) |
| hGH | Human growth hormone |
| hGHR | Human growth hormone receptor |
| Growth Hormone | GHRH1-29 |
| Releasing Hormone | |
| Growth Hormone | GHRP-1, GHRP-2, GHRP-6 |
| Releasing Peptides | |
| IFN | Interferon |
| Ig | Immunoglobulin |
| IGF-1 | Human insulin-like growth factor 1 (IGF-1); somatomedin C |
| IGFBP-3 | Insulin-like growth factor binding protein-3 (IGFBP3); growth |
| | hormone dependent binding protein BP53; IBP-3; IBP3. |
| IL | Interleukin |
| Imaging agents | ^{99m}-Tc-HYNIC, ^{99m}-Tc-DTPA, ^{99m}Tc-ciprofloxacin, ^{99m}Tc- |
| | methylene diphosphonate (MDP), indium or technetium-^{99m}hexamethylpropylene |
| | amine oxime (HMPAO), ¹¹¹In-DTPA- |
| | Folate, ^{99m}Tc diadenosine tetraphosphate (Ap4A; AppppA, |
| | P1,P4-di(adenosine-5*)-tetraphosphate) and its analog ^{99m}Tc |
| | AppCHClppA, ^{99m}Tc-HYNIC-IL-8., ^{99m}Tc-TRODAT-1, ^{99m}Tc-M- |
| | TRODAT, ^{99m}Tc-RP128, Gd(III) DOTA, ^{99m}Tc-HYNIC-folate, |
| | [^{99m}Tc(SG38)(tricine)(TPPTS)] (RP517), natCu-DOTADY1- |
| | TATE and DOTA-natl-DY1-TATE, ⁶¹Cu-DOTA-DY1-TATE, |
| | ^{99m}Tc labeled [(N-[2-((39-N9-propyl-[3,3,1]aza-bicyclononan- |
| | 3a-yl)(20-methoxy-5-methyl-phenylcarbamate) |
| | (2-mercaptoethyl)amino)acetyl]-2-aminoethanethiolato] |
| | technetium(V) oxide),, Albumin-(biotin) 10-(gadopentetate)25,, |
| | ^{99m}TcO(BAT-NI), (¹³¹1, ¹²³1, ″′In, (⁶⁷Ga, ⁶⁶Ga, ⁶⁴Cu, ⁶⁷Ga- |
| | deferoxamine-folate, ¹¹¹In-octadentate-DTPA-folate (g), ¹¹¹In- |
| | DTPA-NOON-pteroate., ^{99m}Tc(CO)3-DTPA-folate (g), ^{99m}Tc- |
| | EC20 (^{99m}Tc-Cys-Asp-Dap-D-Glu-Pte), ^{99m}Tc-oxa-PnAO-(a |
| | and g)-folate1155, ^{99m}Tc-ethylene dicysteine (^{99m}Tc-L, L-EC), |
| | Co-⁵⁶, Co-⁵⁷, Co-⁵⁸, and Co-⁶⁰-labelled vitamin B12, DCTA, |
| | ¹¹¹Indium-DTPA-vitamin B12 |
| Immunosuppresants | Cyclosporin |
| Inhibitors of | Curcumin and derivatives |
| Angiogenesis | |
| Langerin | Langerhans cell specific c-type lectin; CLEC4K; CD 207 |
| LHRH analog | ANTIDE, luprolide, buserelin, nafarelin |
| LIF | Leukemia inhibitory factor; leukocyte migration inhibitory |
| | factor; leukocyte inhibitory factor; differentiation-stimulating |
| | factor; differentiation-inducing factor (DIF); D factor; |
| | Melanoma-derived LPL inhibitor (MLPLI); Emfilermin; |
| | hepatocyte-stimulating factor (HSF-II HSF-III) |
| L-Selectin | L-selectin; Selectin-L; SELL; leukocyte adhesion molecule-1 |
| | (LAM-1, LAM1); leukocyte cell adhesion molecule-1 (LECAM- |
| | 1); lymph node homing receptor (LNHR); lymphocyte |
| | adhesion molecule 1 (LYAM1); Leu-8; Leu-8/TQ-1 antigen; |
| | leukocyte homing receptor (LHR, hLHRc); LEM1; LSEL; |
| | PLNHR; gp90-mel; DREG antigen; MEL-14 antigen; CD62L |
| Lymphotoxin-a | Lymphotoxin alpha (LTA); lymphotoxin a; tumour necrosis |
| | factor superfamily I (TNFSFI); TNF (lymphocyte derived); |
| | TNFB; TNF β; Coley's toxin; CTX (cytotoxin); DIF |
| | (differentiation inducing factor); F-1 (factor-1); hemorrhagic |
| | factor; necrosin; NKCF (natural killer cytotoxic factor); NK-CIA |
| | (Natural killer colony-inhibiting activity) |
| MC-148 | MC148; MC148P; MC148R (gene symbol); MCC1; v-MCC-1; |
| | v-MCC-I; putative CC chemokine |
| MCP-1 | Monocyte chemoattractant protein-1 (MCP1); small inducible |
| | cytokine A2 (SCYA2); monocyte chemotactic and activating |
| | factor (MCAF); monocyte chemotactic protein 1 (MCP-1); |
| | smooth muscle cell chemotactic factor (SMC-CF); lymphocyte- |
| | derived chemotactic factor (LDCF); glioma-derived monocyte |
| | chemotactic factor (GDCF); tumor-derived chemotactic factor |
| | (TDCF); monocyte secretory protein JE; HC-11 |
| MIP-1a | Macrophage inflammatory protein 1 alpha (MIP1A, MIP-1A, |
| | MIP-1a); chemokine CC motif; ligand 3; CCL3; Tonsillar |
| | lymphocyte LD78 alpha protein; G0/G1 switch regulatory |
| | protein 19-1; G0S19-1 protein; SIS-beta; PAT 464.1; small |
| | inducible cytokine A3; SCYA3; SYO3 |
| Melanoma treatment | Genistein, Platinum drugs Cisplatin, oxaliplatin, carboplatin, |
| | diaminocyclohexylplatinin (DACH-platinum), nedaplatin, |
| | cycloplatan, SKI2053R, Loboplatin, enloplatin, zeniplatin, |
| | Miboplatin, L-NDDP, TRK-710, MBA (bis monobromo acetato |
| | trans 1,2-diaminocyclohexane platinum II), PYP (Bis-pyruvato |
| | 1,2-diaminocyclohexaneplatinum II), Sulphato 1,2 |
| | diaminocyclohexane platinum II, Malonato 1,2 |
| | diaminocyclohexane platinum II, Isocitrato 1,2- |
| | diaminocyclohexane platinum II, 4-Carboxyphtalato 1,2- |
| | diaminocyclohexane platinum II, bis-monobromoacetato trans |
| | 1,2-diaminocyclooctane platinum II, 1,1 diaminomethyl |
| | cyclohexane sulphate platinum II, JM-216 (bis-acetato- |
| | ammine-dichloro-cyclohexylamine platinum IV, Iproplatin (cis- |
| | dichloro-trans-dihydroxy-bis-isopropylamino platinum IV, |
| | CHIP, JM-9), Iproplatin (cis-dichloro-trans-dihydroxy-bis- |
| | isopropylamino platinum IV, CHIP, JM-9), curcumin |
| | derivatives, Imiquimod, Calcitriol, Temozolomide, Interferon |
| | Beta, Betulinic Acid |
| MIP-1b | Macrophage inflammatory protein 1 beta (MIP-1 beta, MIP-1β |
| | MIP1β); SCYA4; chemokine CCL-4; chemokine (C—C motif) |
| | ligand 4; CCL4; Immune activation 2; ACT2; Lymphocyte |
| | activation gene 1 (LAG1); AT744.1; Small inducible cytokine |
| | A4; T cell activation protein 2; PAT 744; H400; SIS gamma |
| | Lymphocyte activation gene 1; HC21; G-26 T lymphocyte |
| | secreted protein; ACT-2; Lag-1; HC-21; G26 |
| MLIF | Monocyte Locomotion Inhibitory Factor |
| NSAID | Non-steroidal anti-inflammatory drugs |
| NGF | Nerve growth factor |
| NGFR | Nerve growth factor receptor; p75 NGFR; Gp80-LNGFR; p75 |
| | ICD; low affinity neurotrophin receptor; p75 neurotrophin |
| | receptor (p75 NTR); tumour necrosis factor receptor |
| | superfamily member 16 (TNFRSF16) |
| NO | Nitric oxide |
| Nitric Oxide | iNOS, nNOS, eNOS |
| Synthetase | |
| NOS inhibitors | Niacinamide, vitamin B12 and derivatives, curcumin and |
| | derivatives L-NAME, N^{G}-nitro-L-arginine methyl ester; L-NMA, |
| | N^{W}-methyl-L-arginine, L-NMMA, N^{G}-monomethyl-L-arginine, S- |
| | Methylisothiourea sulphate, Aminoguanidine. 7-nitro indazole |
| | Inhibitro of nNOS, N-nitro-L-arginine |
| Nucleic acids | Anti-sense and RNAi molecules, effector RNA molecules, |
| | virosomes, DNA |
| OSM | Human oncostatin M |
| OX-40 | ACT-35; CD134; Tumor Necrosis Factor Receptor |
| | Superfamily member 4 (TNFRSF4); tax-transcriptionally |
| | activated glycoprotein 1 receptor (TXGP1L) |
| PDGF | Platelet Derived Growth Factor |
| Proton pump | Nexium, omeprazole, Prevacid, Protonix |
| inhibitors | |
| Prostate Cancer | LHRH and analogs, Platinum drugs Cisplatin, oxaliplatin, |
| treatment agents | carboplatin, diaminocyclohexylplatinin (DACH-platinum), |
| | nedaplatin, cycloplatan, SKI2053R, Loboplatin, enloplatin, |
| | zeniplatin, Miboplatin, L-NDDP, TRK-710, MBA (bis |
| | monobromo acetato trans 1,2-diaminocyclohexane platinum |
| | II), PYP (Bis-pyruvato 1,2-diaminocyclohexaneplatinum II), |
| | Sulphato 1,2 diaminocyclohexane platinum II, Malonato 1,2 |
| | diaminocyclohexane platinum II, Isocitrato 1,2- |
| | diaminocyclohexane platinum II, 4-Carboxyphtalato 1,2- |
| | diaminocyclohexane platinum II, bis-monobromoacetato trans |
| | 1,2-diaminocyclooctane platinum II, 1,1 diaminomethyl |
| | cyclohexane sulphate platinum II, JM-216 (bis-acetato- |
| | ammine-dichloro-cyclohexylamine platinum IV, Iproplatin (cis- |
| | dichloro-trans-dihydroxy-bis-isopropylamino platinum IV, |
| | CHIP, JM-9), Iproplatin (cis-dichloro-trans-dihydroxy-bis- |
| | isopropylamino platinum IV, CHIP, JM-9) |
| RANTES | Regulated on Activation, Normal T Cell Expressed and |
| | Presumably Secreted (RANTES); SIS-delta; Small Inducible |
| | Cytokine A5; SCYA5; eosinophil chemotactic polypeptide |
| | (EoCP-1); T cell-specific protein p228; TCP228; Chemokine |
| | CC motif ligand 5 (CCL5) |
| Targeting agents | Amphiregulin, Antibodies including Avastin (bevacizumab), |
| | BEC2 (mitomomab), Tositumomab (Bexxar), Campath |
| | (alemtuzumab), CeaVab, herceptin (trastuzumab), IMC-C225 |
| | (centuximab), Lymphocide (epratuzumab), MDX-210, Mylotarg |
| | (Gemtuzumab), Panorex (Edorcolomab), Rituxan (Rituximab), |
| | Theragyn (pemtumomab), Zamyl, Zevalin (Ibritumomab),, |
| | BAFF, BDNF, bFGF, BMP, BMP-4, BMP-7, CD209L, CSF, |
| | EGF, Fas-Ligand, Fc fragments, FGF, FGF2, FGFR1, FGFR4, |
| | Flt3, Flt3-Ligand, G-CSF, GM-CSF, hGH, hGHR, Growth |
| | Hormone Releasing Hormone and analogs, Growth Hormone |
| | Releasing Peptides, IFN, IgG, IGF-1, IGFBP-3, Interleukins, |
| | Langerin, LHRH analogs, LIF, L-Selectin, Lymphotoxin-a, MC- |
| | 148, MCP-1, MIP-1a, MIP-1b, MLIF, NGF, NGFR, OSM, OX- |
| | 40, PDGF, RANTES, Tf, TGF, TGF-b1, TNF, TNF-a, TPO, |
| | TrkA, TrkB, VEGF, Vitamin derivatives, including derivatives of |
| | vitamin B12, biotin, folate and riboflavin |
| Testicular cancer | Carboplatin |
| treatment agents | |
| Tissue remodelling | Botox (Clostridium botulinum toxin) |
| molecules | |
| Tf | Transferrin |
| TfR | Transferrin receptor |
| TGF | Transforming Growth Factor |
| TGF-b1 | Transforming growth factor beta 1 (TGFB1); TGF-beta 1; |
| | Transforming growth factor B1; TGFB; TGF-b1; cartilage |
| | inducing factor A (CIF-A); differentiation-inhibiting factor (DIF); |
| | epithelial growth inhibitor (EGI); Epstein-Barr virus inducing |
| | factor (EIF); glioma-derived T-cell suppressor factor (G-TsF); |
| | immunosuppressive factor (ISF); milk-derived growth factor |
| | (MDGF); milk growth factor-b (MGF-b); platelet-derived |
| | endothelial cell growth inhibitor (PDGI); Polyergin; Simian |
| | BSC-1 cell growth inhibitor; transformed cell growth factor |
| | (TCGF); tissue-derived growth inhibitor (TGI); tumor inducing |
| | factor-1 (TIF-1); CED; DPD1 |
| TGF-bR2 | Transforming growth factor beta receptor 2; TGFBRII; TGF- |
| | beta receptor type II; EC 2.7.1.37; TGFR-2; HNPCC6; RIIC |
| TNF | Tumor Necrosis Factor |
| TNF-a | Tumor necrosis factor (TNF); tumor necrosis factor ligand |
| | superfamily member 2 (TNFRSF2); TNF-alpha; TNF-a; TNF-a; |
| | TNFA; TNF (monocyte derived); TNF (macrophage derived); |
| | DIF; cachectin |
| TNFR | Tumor Necrosis Factor Receptor |
| TNFR1 | Tumor necrosis factor receptor 1 (TNFRI); TNF-RI; TNFR1; |
| | TNF-R1; TNFAR; CD120a; p55; p60; TNF receptor |
| | superfamily member 1A (TNFRSF1A) |
| TNFR2 | Tumor necrosis factor receptor type II (TNFRII, TNF-RII); |
| | TNFR2; TNF-R2; CD120b; p75; p80; TNF-alpha receptor; |
| | TNFBR; TNF receptor superfamily member 1B (TNFRSF1B); |
| | commercially available TNF receptor Fc fusion proteins such |
| | as etanercept |
| TPO | Thrombopoietin (TPO); thrombocytopoietin; megakaryocyte |
| | colony stimulating factor (MKCSF); cellular homologue of the |
| | murine myeloproliferative leukemia virus oncogene ligand (c- |
| | Mpl ligand, MPL ligand) ML; MPLLG; megakaryocyte growth |
| | and development factor (MGDF); THPO (gene name) |
| TrkA | Neurotrophic tyrosine kinase receptor type 1; TRK1 |
| | transforming tyrosine kinase protein; p140-TrkA; neurotrophic |
| | tyrosine kinase receptor |
| TrkB | Neurotrophic tyrosine kinase receptor type 2; NTRK2; GP145- |
| | TrkB |
| VEGF | Vascular endothelial growth factor |
| PDE5 inhibitors | phosphodiesterases type 5 inhibitors; sildenafil, tadalafil, |
| | vardenafil |
| Vitamin B12 | See AdeCbl |
| derivatives | |
| Weight Modifying | Insulin |
| drugs | |

### BRIEF DESCRIPTION OF THE FIGURES

FIG. 1 is a photographic representation showing penetration of rhodamine-labeled BSA (red) into a hair follicle and extending to the bottom of the follicle.

FIG. 2 is a photographic representation showing penetration of rhodamine-labeled BSA (red) into the follicle-associated sebaceous gland.

FIG. 3 is a representation showing the distribution of EGF in mice following transdermal application of EGF in a microemulsion formulation after 3 hours.

FIG. 4 is a representation demonstrating the distribution of insulin in mice following transdermal application of insulin in a microemulsion formulation overnight.

FIG. 5 is a graphical representation showing the distribution of TNFR2-Fc fusion protein in mice following transdermal application of TNFR2-Fc in a microemulsion formulation after 3 hours.

FIG. 6 is a graphical representation showing the time-course of rear foot thickness of a C57/BI6 strain mouse following Carrageenan-induced inflammation in the right rear foot. Topically administered pharmaceutical composition(s) containing containing VB12 analogs and/or curcumin analogs were active in reducing inflammation within the right rear foot. Microemulsion (ME) alone, left foot (open circles); ME alone, right foot (solid circles); ME containing AdeCbl, right foot (open diamonds); ME containing TTC, right foot (crosses); ME containing AdeCbl and TTC, right foot (open diamonds); AdeCbl=adenosyl-cobalamin; TTC=tetrahydrocurcumin.

FIG. 7 is a graphical representation showing the time-course of increase in the rear foot thickness of a C57/BI6 strain mouse following Carrageenan-induced inflammation in the right rear foot. Topically administered pharmaceutical composition(s) containing various Vitamin B12 (VB12) analogs were active in reducing inflammation within the right reat foot. Microemulsion (ME) alone, left foot (open circles); ME alone, right foot (solid circles); ME containing Cbl, left foot (open triangles); ME containing Cbl, right foot (solid triangles); ME containing VB12-CN, right foot (crosses); ME containing OH-VB12, right foot (open diamonds); ME containing co-enzyme VB12, left foot (open squares); ME containing co-enzyme VB12, right foot (solid squares). VB12-CN=cyanocobalamin; OH-VB12=hydroxycobalamin; Cbl=cobalamin.

FIG. 8 is a graphical representation showing the time-course of rear foot thickness of a C57/BI6 strain mouse following Carrageenan-induced inflammation in the right rear foot. Topically administered pharmaceutical composition(s) containing MLIF molecules or VB12 analogs were active in reducing inflammation within the right rear foot. AdeCbl=adenosyl-cobalamin; Microemulsion (ME) alone, left foot (open circles); ME alone, right foot (solid circles); ME containing AdeCbl, right foot (open diamonds); ME containing MLIF-A, right foot (solid squares); ME containing MLIF-B, right foot (open squares); ME containing MLIF-C, right foot (crosses).

FIG. 9 is a graphical representation showing the time-course of rear foot thickness of a BAlb/C strain of mouse following Carrageenan-induced inflammation in the right rear foot. Topically administered pharmaceutical compositions contained either recombinant human TNF receptor II-IgG1 Fc chimera (5 mg/ml) or adalimumab, a commercially available anti-TNF antibody; Humira®; Abbott; 4 mg/ml). Microemulsion (ME) alone, left foot (open circles); ME alone, right foot (solid circles); ME containing recombinant human TNF receptor II-IgG1 Fc chimera, right foot (open triangles); ME containing adalimumab, right foot (solid triangles). Both pharmaceutical compositions were active in reducing inflammation within the right rear foot.

FIG. 10 is a graph showing the modification in the weight of skin, muscle and visceral fat in overweight Swiss mice who have been topically or subcutaneously administered with GRHP-6, Insulin-like Growth Factor 1 (IGF1) or Insulin.

FIG. 11 is a graphical representation showing the time course of weight loss in overweight Swiss mice who have been topically administered with GRHP-6, or Insulin-like Growth Factor 1 (IGF1), or Insulin.

FIG. 12 is a graph showing the development of an antibody response in Balb/C and C57BI mice topically vaccinated with tetanus toxiod. The species differences in antibody responses to topical and intramuscular administration of the antigen are evident.

FIG. 13 is a graph depicting the development of a T cell response in Balb/C and C57BI mice topically vaccinated with tetanus toxiod. The species differences in cellular responses to topical and intramuscular administration of the antigen, as determined by measurement of footpad thickness, are evident.

### DETAILED DESCRIPTION OF THE INVENTION

It is to be understood that unless otherwise indicated, the subject invention is not limited to specific formulations, manufacturing methods, diagnostic methods, assay protocols, nutritional protocols, or research protocols or the like as such may vary. It is also to be understood that the terminology used herein is for the purpose of describing particular embodiments only and is not intended to be limiting.

It must be noted that, as used in the subject specification, the singular forms “a”, “an” and “the” include plural aspects unless the context already dictates otherwise. Thus, for example, reference to “a pharmaceutically active agent” includes a single pharmaceutically active agent as well as two or more pharmaceutically active agents, “an agent” includes a single agent as well as two or more agents; reference to “the microemulsion” includes one or more microemulsions; and so forth.

The terms “compound”, “active agent”, “chemical agent”, “pharmaceutical agent”, “pharmacologically active agent”, “medicament”, “active” and “drug” are used interchangeably herein to refer to a chemical compound and in particular a protein or a chimeric molecule thereof that induces a desired pharmacological and/or physiological effect. The terms also encompass pharmaceutically acceptable and pharmacologically active ingredients of those active agents specifically mentioned herein including but not limited to salts, esters, amides, prodrugs, active metabolites, analogs and the like. When the terms “compound”, “active agent”, “chemical agent”, “pharmaceutical agent”, “pharmacologically active agent”, “medicament”, “active” and “drug” are used, then it is to be understood that this includes the active agent per se as well as pharmaceutically acceptable, pharmacologically active salts, esters, amides, prodrugs, metabolites, analogs, etc. The desired pharmacological effect is dependent on the agent used but includes an anti-inflammatory effect, an immune response, a change in the level of gene expression, a pigmentation effect including a lightening effect, an imaging effect on a target such as a target lymph node, an anti-muscle dystrophic effect, weight loss, treatment of cancer, reduction in pain or a physiological effect such as correction of erectile dysfunction.

Reference to a “compound”, “active agent”, “chemical agent”, “pharmaceutical agent”, “pharmacologically active agent”, “medicament”, “active” and “drug” includes combinations of two or more active agents such as two or more proteins. A “combination” also includes multi-part such as a two-part composition where the agents are provided separately and given or dispensed separately or admixed together prior to dispensation.

The terms “agents” and the like include peptides, polypeptides and proteins, large and small chemical molecules, hormones, steroids and nucleic acid molecules as well as viral components such as virosomes.

The terms “effective amount” and “therapeutically effective amount” of an agent as used herein mean a sufficient amount of the pharmaceutical agent, alone or in combination with other agents to provide the desired therapeutic or physiological effect or outcome. Undesirable effects, e.g. side effects, are sometimes manifested along with the desired therapeutic effect; hence, a practitioner balances the potential benefits against the potential risks in determining what is an appropriate “effective amount”. The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation.

An “effective amount”, for example, would include an amount of a cytokine or anti-cytokine antibody or anti-sense or RNAi molecule directed to a gene or mRNA transcript encoding an inflammatory cytokine to inhibit an inflammatory response by a reduction in aberrant skin proliferation, antibody production, cytokine production and/or immune response effector molecule production.

By “pharmaceutically acceptable” carrier, excipient or diluent is meant a pharmaceutical vehicle comprised of a material that is not biologically or otherwise undesirable, i.e. the material may be administered to a subject along with the selected active agent without causing any or a substantial adverse reaction. Carriers may include excipients and other additives such as diluents, detergents, coloring agents, wetting or emulsifying agents, pH buffering agents, preservatives, and the like.

Similarly, a “pharmacologically acceptable” salt, ester, amide, prodrug or derivative of a compound as provided herein is a salt, ester, amide, prodrug or derivative that this not biologically or otherwise undesirable.

The terms “treating” and “treatment” as used herein refer to reduction in severity and/or frequency of symptoms of the condition being treated, elimination of symptoms and/or the underlying cause, prevention of the occurrence of symptoms of the condition and/or their underlying cause and improvement or remediation or amelioration of damage following a condition.

“Treating” a subject may involve prevention of a condition or other adverse physiological event in a susceptible individual as well as treatment of a clinically symptomatic individual by ameliorating the symptoms of the condition.

A “subject” as used herein refers to an animal, in a particular embodiment, a mammal and in a further embodiment human who can benefit from the pharmaceutical formulations and methods of the present invention. There is no limitation on the type of animal that could benefit from the presently described pharmaceutical formulations and methods. A subject regardless of whether a human or non-human animal may be referred to as an individual, patient, animal, host or recipient. The compounds and methods of the present invention have applications in human medicine, veterinary medicine as well as in general, domestic or wild animal husbandry including agriculturally-relevant animals (e.g. livestock animals).

As indicated above, in a particular embodiment, the animals are humans or other primates such as orangutans, gorillas or marmosets, further contemplated are livestock animals, laboratory test animals, companion animals or captive wild animals, as well as avian species.

Examples of laboratory test animals include mice, rats, rabbits, guinea pigs and hamsters. Rabbits and rodent animals, such as rats and mice, provide a convenient test system or animal model. Livestock animals include sheep, cows, pigs, goats, horses, yaks, Ilamas, camels and donkeys. Companion animals include dogs and cats. Non-mammalian animals such as avian species, fish and amphibians.

The term “protein” is used interchangeably with the term “peptide” or “polypeptide” and is used in its most general sense including ligands and receptors. As used herein, reference to a particular protein should be understood to refer to the “complete” protein as well as fragments, isoforms, derivatives or homologs or chimeras thereof comprising one or more amino acid additions, deletions or substitutions, but which substantially retain the biological activity of the complete protein.

The term “polypeptide” refers to a polymer of amino acids and its equivalent but does not imply a limitation as to a specific length of the product, thus, peptides, oligopeptides, polypeptides and proteins are included within the definition of a “polypeptide”. This term also includes all co- or post-translationally modified forms of a polypeptide. Also included within the definition are, for example, polypeptides containing one or more analogs of an amino acid including, unnatural amino acids or polypeptides with substituted linkages.

As used herein, the terms “co- or post-translational modifications” refer to covalent modifications occurred during or after translation of the peptide chain. Exemplary co- or post-translational modifications include, but are not limited, to acylation (including acetylation), amidation or deamidation, biotinylation, carbamylation (or carbamoylation), carboxylation or decarboxylation, disulfide bond formation, fatty acid acylation (including myristoylation, palmitoylation and stearoylation), formylation, glycation, glycosylation, hydroxylation, incorporation of selenocysteine, lipidation, lipoic acid addition, methylation, N- or C-terminal blocking, N- or C-terminal removal, nitration, oxidation of methionine, phosphorylation, proteolytic cleavage, prenylation (including farnesylation, geranyl geranylation), pyridoxal phosphate addition, sialylation or desialylation, sulfation, ubiquitinylation (or ubiquitination) or addition of ubiquitin-like proteins.

A “derivative” of a polypeptide of the present invention also encompasses a portion or a part of a full-length parent polypeptide, which retains partial transcriptional activity of the parent polypeptide and includes a variant. Such “biologically-active fragments” include deletion mutants and small peptides, for example, for at least 10, in a particular embodiment, at least 20 and in a further embodiment at least 30 contiguous amino acids, which exhibit the requisite activity. Peptides of this type may be obtained through the application of standard recombinant nucleic acid techniques or synthesized using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines” edited by Nicholson and published by Blackwell Scientific Publications. Alternatively, peptides can be produced by digestion of an amino acid sequence of the invention with proteinases such as endoLys-C, endoArg-C, endoGlu-C and staphylococcus V8-protease. The digested fragments can be purified by, for example, high performance liquid chromatographic (HPLC) techniques. Any such fragment, irrespective of its means of generation, is to be understood as being encompassed by the term “derivative” as used herein.

The pharmaceutical agent of the present invention may be a chimeric molecule comprising an isolated protein or a fragment thereof, such as an extra-cellular domain of a membrane bound protein, linked to the constant (Fc) or framework region of a human immunoglobulin via one or more protein linkers. Such a chimeric molecule is also referred to herein as protein-Fc. Other chimeric molecules contemplated by the present invention include the protein or protein-Fc or a fragment thereof, linked to a lipid moiety such as a polyunsaturated fatty acid molecule. Such lipid moieties may be linked to an amino acid residue in the backbone of the molecule or to a side chain of such an amino acid residue. The human immunoglobulin may be selected from IgG1, IgG2, IgG3, IgG4, IgA1, IgA2, IgM, IgE, IgD.

The pharmaceutical agent of the present invention may further include a chimeric molecule comprising an isolated protein or a fragment thereof, such as an extra-cellular domain of a membrane bound protein, linked to the constant (Fc) or framework region of a mammalian immunoglobulin via one or more protein linker. In another aspect, the mammal Fc or framework region of the immunoglobulin is derived from a mammal selected from the group consisting of primates, including humans, marmosets, orangutans and gorillas, livestock animals (e.g. goats, cows, sheep, pigs, horses, donkeys, asses), laboratory test animals (e.g. mice, rats, guinea pigs, hamsters, rabbits, companion animals (e.g. cats, dogs) and captured wild animals (e.g. rodents, foxes, deer, kangaroos). In another embodiment the Fc or framework region is a human immunoglobulin. In a particular embodiment the mammal is a human. Such a chimeric molecule is also referred to herein as protein-Fc. Other chimeric molecules contemplated by the present invention include the protein or protein-Fc or a fragment thereof linked to a lipid moiety such as a polyunsaturated fatty acid molecule. Such lipid moieties may be linked to an amino acid residue in the background of the molecule or to a side chain of such an amino acid residue.

The pharmaceutical agent has one or more of the following activities: anti-infective, anti-inflammatory, anti-tumor, skin repair, skin damage prevention, skin anti-aging, skin lightening or whitening, hair growth promotion or hair growth inhibition, muscle repair, weight loss promoting, erectile dysfunction correcting, anti-acne and CNS healing activities.

Examples of Particular Agents and Applications Follow:

1. Anti-inflammatory Activities for the Treatment of Skin Disorders.

The microemulsions of the present invention are useful inter alia for delivering small molecules, proteins (including peptides and polypeptides and chimeras), antibodies, other water soluble molecules as well as oil-soluble molecules useful in treating skin-related inflammatory conditions. For example, the microemulsions can be used to deliver nitric oxide and TNF alpha inhibitors. Vitamin B12, for example, is a useful nitric oxide chelator and is useful in reducing skin-related inflammatory responses. In a another embodiment, the present invention contemplates a topical composition comprising a vitamin (VB12) derivative (or analogs thereof for the treatment of skin-related inflammatory conditions. Any inhibitor of i-nitric oxide synthase, e-nitric oxide synthase or n-nitric oxide synthase is contemplated by the present invention. Nicotinamide may also be used to block nitric oxide synthase. Other agents useful for treating psoriasis, dermatitis and itch include curcuminoids, COX-1 and COX-2 inhibitors, vitamins such as vitamin E and vitamin C. In a related embodiment, the present invention contemplates a topical composition comprising a curcuminoid derivative (or an analog thereof) for the treatment of inflammatory conditions of the skin.

Proteins useful for treating skin-related inflammatory conditions such as psoriasis, dermatitis, systemic lupus erythematosus (SLE) and itch include antibodies directed against IL-20, C5aR, IFN alpha, TNF (e.g. infliximab, adalimumab), IL-12, IL-1, IL-6, leukotrienes and IL-4. Such proteins include receptor-Fc chimeras, for example, TNF receptor-Fc chimera proteins such as etanercept.

In a further embodiment, the vitamin B12 derivative is adenosylcobalamin and the curcuminoid is tetrahydrocurcumin.

In yet another embodiment, an anti-TNF molecule is administered within a microemulsion containing both adenosylcobalamin and tetrahydrocurcumin.

2. Anti-inflammatory Activities for the Treatment of Bruising.

The microemulsions of the present invention are useful inter alia for delivering small molecules, proteins (including peptides and polypeptides and chimeras), antibodies, other water soluble molecules as well as oil-soluble molecules useful in treating bruising. This approach avoids the toxicity common with systemic or oral delivery of, for example, anti-ecchymosis therapies such as extracts of Arnica montana for the treatment of bruising. For example, the microemulsions can be used to deliver nitric oxide and TNF alpha inhibitors to a site of ecchymosis following cosmetic surgery such as rhytidectomy. Vitamin B12, for example, is a useful nitric oxide chelator and is useful in reducing bruising, also known as ecchymosis. In one embodiment, the present invention contemplates a topical composition comprising a vitamin B12 derivative (or analogs thereof) for the treatment of bruising. In a further embodiment, the present invention contemplates a topical composition comprising a vitamin B12 derivative (or an analog thereof and a curcuminoid derivative (or an analog thereof) for the treatment of bruising. In a related embodiment the vitamin B12 derivative is adenosylcobalamin and the curcuminoid is tetrahydrocurcumin.

3. Anti-inflammatory Activities for the Treatment of Muscular or Tendon Damage.

The microemulsions of the present invention are useful inter alia for delivering small molecules, proteins (including peptides and polypeptides and chimeras), antibodies, other water soluble molecules as well as oil-soluble molecules useful in treating musclar or tendon damage. Conditions such as delayed onset muscle soreness (DOMS) can prevent athletes from recovering following a training event or game, resulting in measurable reductions in power, flexibility or endurance. Vitamin B12, for example, is a useful nitric oxide chelator and is useful in reducing DOMS. The microemulsions of the present invention can be used to deliver nitric oxide and TNF alpha inhibitors to muscular tissue to ameliorate conditions such as DOMS. This approach avoids the toxicity common with systemic or oral delivery of other anti-inflammatory therapies such as COX inhibitors. In a related embodiment, the present invention contemplates a topical composition comprising a vitamin B12 derivative (or analogs thereof for the treatment of muscular damage such as DOMS. Additionally such treatment also be used for tendon injuries such as tenosynovitis, Carpal Tunnel syndrome, bruising, bursitis and pain, repetitive strain injury (RSI), patellotendonitis, paratendonitis, proliferative tendonitis, degenerative tendonitis, enthesis In more particular embodiment, the present invention contemplates a topical composition comprising a vitamin B12 derivative (or an analog thereof and a curcumioid derivative (or an analog thereof) for the treatment of muscular damage such as DOMS.

In a further embodiment the vitamin B12 derivative is adenosylcobalamin and the curcuminoid is tetrahydrocurcumin.

4. Anti-inflammatory Activities for the Treatment of Joint Damage.

The microemulsions of the present invention are useful inter alia for delivering small molecules, proteins (including peptides and polypeptides and chimeras), antibodies, other water soluble molecules as well as oil-soluble molecules useful in treating joint damage. Joint damage may arise through chronic inflammatory disease states such as rheumatoid arthritis and systemic lupus erythematosus (SLE) or in response to acute damage such as that experienced by individuals during sporting activities (e.g. ankle sprain). Vitamin B12, for example, is a useful nitric oxide chelator and is useful in reducing joint-related inflammation. The microemulsions of the present invention can be used to deliver nitric oxide and TNF alpha inhibitors to joint tissue to ameliorate conditions such as rheumatoid arthritis and joint damage following acute injury. This approach avoids the toxicity common with systemic or oral delivery of other anti-inflammatory therapies such as COX inhibitors. In a related embodiment, the present invention contemplates a topical composition comprising a vitamin B12 derivative (or analogs thereof for the treatment of rheumatoid arthritis, SLE, or acute joint damage. In a further preferred embodiment, the present invention contemplates a topical composition comprising a vitamin B12 derivative (or an analog thereof and a curcuminoid derivative (or an analog thereof for the treatment of rheumatoid arthritis, SLE, or acute joint damage,

In a related embodiment the vitamin B12 derivative is adenosylcobalamin and the curcuminoid is tetrahydrocurcumin.

5. Topical Vaccines.

The microemulsions are useful as topical vaccines such as to induce an antibody response and/or a cytotoxic T-cell and/or a T helper response including a TH1/TH2 response. Topical vaccines are particularly useful for mobilizing dendritic cells in the dermal layer and increasing numbers of activated dendritic cells. In the past, hyperdermic needles were able to deliver agents to the subcutaneous layer or muscle layer, however, the procedure was invasive and did not necessarily target the desired region. The topical microemulsion may be provided with or without an adjuvant. Adjuvants contemplated herein include microorganisms or viruses or components thereof and chemical substances which enhance the immune response and/or chemical which act as preservatives and tissue fixatives. Particularly useful adjuvants include aluminium hydroxide, aluminium phosphate and calcium phosphate. In addition, oil based emulsions and products from bacteria including synthetic derivatives and liposomes of gram-negative bacteria, endotoxins, cholesterol, fatty acids, aliphatic amines, parafinic and vegetable oils, monophosphoryl lipid A, iscoms with Quil-A and Syntex agents formulations containing threonyl derivaties or muramyl type peptides may be employed. Other adjuvants contemplated by the present invention include Freund's emulsify oil adjuvants (complete and incomplete), ARLACEL A, mineral oil, emulsified peanut oil adjuvant (adjuvant 65), mineral compounds and bacterial products from Bordetella pertussis, Corynebacterium granulosom-derived P40 component, lipopolysaccaride, Mycobacterium or components thereof, inulin, cholera toxin and/or liposomes may also be employed. Specific disease states that can be topically treated with a microemulsion of the present invention include Hepatitis B virus (HBV), Hepatitis C virus (HCV), DPT, Human paploma virus (HPV), Japanese encephalitis, shigella, malaria, rabies, measles, mumps, rubella, whooping cough, rotavirus and herpes zoster. In a related embodiment, the present invention contemplates a topical composition comprising a antigen and an adjuvant within a microemulsion for the vaccination of a vertebrate host.

6. Topical Fat Weight Loss.

The microemulsions of the present invention may be used to induce weight loss via lymphodystrophy. It is proposed that topically applied insulin induces insulin-mediated lymphodystrophy. There is minimal if any entry of the insulin to the blood system and hence no increase in glucose levels. Reference to “insulin” includes insulin analogs and precursor forms of insulin which may have the ability to interact with insulin receptors but lack signalling capability. In a related embodiment, the present invention contemplates a topical composition comprising insulin for the amelioration of sub-cutaneuous fat deposits. In another embodiment of the present invention there is described a topical composition comprising growth factor administered within a microemulsion for the amelioration of obesity. In yet another embodiment of the present invention there is described a topical composition comprising a growth hormone releasing hormone, or growth hormone releasing peptide for the amelioration of obesity.

7. Imaging and Treatment of Skin Cancer and Breast Cancer.

According to this aspect of the present invention, the microemulsions are able to access or introduce imaging reporter molecules to the sentinel lymph node in relation to particular cancers such as melanoma and breast carcinoma. The sentinel lymph node is the first lymph node to which cancer is likely to spread from the primary tumor. Cancer cells may appear first in the sentinel node before spreading to other lymph nodes and other places in the body. In accordance with the present invention, imaging prior to the removal of the sentinel lymph node increases the likelihood that the correct lymph node is removed in its entirety. Any imaging agent may be employed, such as a radio-active agents, chelates, fluorescent compounds and metals. The identification of the sentinel lymph node is improved and can be successfully removed thus reducing the risk of spread of a cancer. In chelate methods a radionuclide is bound to the targeting molecule indirectly, through a bifunctional chelating agent (BFCA)

Examples of radioactive agents which may be admixed with the microemulsions include the following: Antimony-124, Antimony-125, Arsenic-74, Barium-103, Barium-140, Beryllium-7, Bismuth-206, Bismuth-207, Cadmium-109, Cadmium-11Sm, Calcium-45, Cerium-139, Cerium-141, Cerium-144, Cesium-137, Chromium-51, Cobalt-56, Cobalt-57, Cobalt-58, Cobalt-60, Cobalt-64, Erbium-169, Europium-152, Gadolinium-153, Gold-195, Gold-199, Hafnium-175, Hafnium0175-181, Indium-111, Iridium-192, Iron-55, Iron-59, Krypton-85, Lead-210, Manganese-54, Mercury-197, Mercury-203, Molybdenum-99, Neodymium-147, Neptunium-237, Nickel-63, Niobium-95, Osmium-185+191, Palladium-103, Platinum-195m, Preseodymium-143, Promethium-147, Protactinium-233, Radium0226, Rhenium-186, Rubidium-86, Ruthenium-103, Ruthenium-106, Scandium-44, Scandium-46, Selenium-75, Silver-110m, Silver-111, Sodium-22, Strontium-85, Strontium-89, Strontium-90, Sulfur-35, Tantalum-82, Technetium-99m, Tellurium-125, Tellurium-132, Thallium-204, Thorium-228, Throium-232, Thallium-170, Tin-113, Titanium-44, Tungsten-185, Vanadium-48, Vanadium-49, Ytterbium-169, Yttrium-88, Yttrium-90, Yttrium-91, Zinc-65 or Zirconium-95.

Examples of chelating groups which may be admixed with the microemulsions include the following:

1,3,5triaminocyclohexane; 1,3,5triaminocyclohexane N-pyridine; 1,1-cyclobutanedicarboxylic acid; 1,2-Dimethyl-3-hydroxypyridin-4-one; 1,2-dimethyl-3-hydroxypyridin-4-one (Deferiprone); 1,4,7,10-tetraaza-4,7,10-tris(carboxymethyl)-1-cyclododecylacetyl-R-(+)-R-methylbenzylamine (DOTA-MBA); 1,4,7,10-tetraazacyclododecane-N,N,N,N-tetraacetic acid (DOTA); 1,6-dimethyl-2-(1-hydroxyethyl)-3-hydroxypyridin-4-one; 1-ethyl-2- (1-hydroxyethyl)-3-hydroxypyridin-4-one; 1-hydroxypryidin-2one; 1-hydroxypryidin-2one; 2-Deoxy-2-(N-carbamoylmethyl-[N9-29-methyl-39-hydroxypyrid-49-one])-D-glucopyranose; 2-furoylcarboxaldehyde isonicotinoyl hydrazone (FIH); 2-hydroxy-1-naphthylaldehyde benzoyl hydrazone; 2-hydroxy-1-naphthylaldehyde isonicotinoyl hydrazone; 2-hydroxy-1-napthylaldehyde isonicotinoyl hydrazone; 2-methyl-3-hydroxy-4H-benzopyran-4-one (MCOH); 2-pyridylcarboxaldehyde 2-thiophenecarboxyl hydrazone (PCTH); 2-pyridylcarboxaldehyde benzoyl hydrazone (PCBH); 2-pyridylcarboxaldehyde isonicotinoyl hydrazone (PCIH); 2-pyridylcarboxaldehyde isonicotinoyl hydrazone (PCIH); 2-pyridylcarboxaldehyde m-bromobenzoyl hydrazone (PCBBH); 2-pyridylcarboxaldehyde m-bromobenzoyl hydrazone (PCBBH); 2-pyridylcarboxaldehyde p-aminobenzoyl hydrazone (PCAH); 2-pyridylcarboxaldehyde p-hydroxybenzoyl hydrazone (PCHH); 2-pyridylcarboxaldehyde thiophenecarboxyl hydrazone (PCTH); 311 2-hydroxy-1-napthylaldehyde isonicotinoyl hydrazone; 3-aminopyridine-2-carboxaldehyde thiosemicarbazone (Triapine®); 3-aminopyridine-2-carboxyaldehyde thiosemicarbazone; 3-hydroxypyridin-2-one; 3-hydroxypyridin-4-one; 4-[3,5-bis-(hydroxyphenyl)-1,2,4-triazol-1-yl]-benzoic acid (ICL670A), aminocarboxylates, BAPTA/AM (1,2-bis(2-aminophenoxy)ethane-N,N,N9,N9-tetraacetic acid acetoxymethyl ester); Catechols; CDTA cyclohexanediaminetetraacetic acid; cis-1,3,5-triaminocyclohexane; clioquinol; DDC diethyldithiocarbamate; Defarasirox; Deferiprone (1,2-dimethyl-3-hydroxypyrindin-4-one); Deferoxamine; Demercaptol; DFO desferrioxamine; DFOA; Diaminocyclohexane; diethylenetriaminepentaacetic acid; DMB; DMPA dimercaptopropionic acid; DMPS; DMSA dimercaptosuccinic acid; DPA (D-penicillamine); DTPA (diethylene triamine pentaacetic acid); EDTA, (ethylendiaminetetraacetic acid); Ferroportin-1; Hydroxamates; Hydroxycarboxylates; hydroxypyridinones; IDA iminodiacetic acid; MECAM;

N,N_-bis-(2-hydroxybenzyl)ethylenediamine-N,N_-diacetic acid (HBED); N,N¢-bis(2-hydroxybenzyl)ethylenediamine-N,N-diacetic acid (H BED); N,N¢-Bis(3,4,5-trimethoxybenzyl)ethylenediamine-N,N-diacetic acid; N,N-dimethyl-2,3-dihydroxybenzoic acid; N4,NR,NR,N_,N_-pentakis[[((N-hydroxy-N-methyl]carbonyl)methyl]-2,6-diamino-4-azahexanoic hydrazide; NAPA N-acetyl-D-penicillamine; N-ethyl N,N,N-tris(pyridylmethyl)-cis,cis, 1,3,5,-triaminocyclohexane; NOTP (1,4,7-triazacyclononane-1,4,7-tris(methylenephosphonate)); NOTPME (1,4,7-triazacyclononane-1,4,7-tris(methylenephosphonatemonoethylester)); N-pyridine; NTA nitrilotriacetic acid; oxalic acid; pyridoxal hydrochloride; pyridoxal isonicotinoyl hydrazone; Pyridoxal isoonicotinoyl hydrazone (PIH); pyridoxal metachlorobenzoyl hydrazone; pyridoxal metafluorobenzoyl hydrazone; pyridoxal paramethoxybenzoyl hydrazone; rhizoferrin; salicylaldehyde benzoyl hydrazone; staphloferrin; staphloferrin; succinic acid; tachpyridine; TETA (triethylenetetra-amine); tetraaza-4,7,10-tris(carboxymethyl)-1-cyclododecylacetylbenzylamine (DOTA-BA); TREN-(Me-3,2-hydroxipyridonate) (HOPO); TRENCAM; Triapine, 3-aminopyridine-2-carboxyaldehyde thiosemicarbazone; TTD tetratethythiuramdisulfide; TTHA triethylenetetraminehexaacetic acid; Deferiprone (1,2-dimethyl-3-hydroxypyridin-4-one); HYNIC (6-hydrazinonicotinamide); HYNIC-Kp-DPPB and HYNIC-Ko-DPPB where (HYNIC) 6-hydrazinonicotinamide, where K is lysine and DPPB is diphenylphosphine-benzoic acid; HPO 3-hydroxypyridin-4-one; 1-(2′-carboxyethyl)-2-methyl-3-hydroxypyridin-4-one; 1-(3′-hydroxypropyl)-2-methyl-3-hydroxypyridin-4-one; or 1-(2′-hydroxyethyl)-2-ethyl-3-hydroxypyridin-4-one.

Chelates for use during imaging are readily formed by mixing the appropriate radionuclide (formulated within the microemulsion) with an equimolar amount of the particular chelating moiety (also formulated within the microemulsion). The resultant radionuclide-chelate forms spontaneously within the microemulsion and can be used almost immediately for topical application.

Microemulsions can also be used to topically introduce chemotherapeutic agents and cytotoxic compounds to a site close to the tumor. This approach avoids the toxicity common with the systemic or oral delivery of most chemotherapies. Particular cancers that may be treated in this way include skin cancers such as melanoma, squamous cell carcinoma, basal cell carcinoma, solar keratosis as well as breast carcinoma.

Examples of chemotherapeutic agents which may be admixed with the microemulsions include the following:

Antimetabolites: substances that interfere with the body's chemical processes, such as creating proteins, DNA, and other chemicals needed for cell growth and reproduction; in cancer treatment, antimetabolite drugs disrupt DNA production, which in turn prevents cell division. Examples include Azaserine, D-Cycloserine, Mycophenolic acid, Trimethoprim, 5-fluorouracil, capecitabine, methotrexate, gemcitabine, cytarabine (ara-C) and fludarabine.

Antitumor antibiotics which interfere with DNA by stopping enzymes and mitosis or altering the membranes that surround cells. These agents work in all phases of the cell cycle. Thus, they are widely used for a variety of cancers. Examples of antitumor antibiotics include dactinomycin, daunorubicin, doxorubicin (Adriamycin), idarubicin, and mitoxantrone.

Mitotic inhibitors which are plant alkaloids and other compounds derived from natural products. They can inhibit, or stop, mitosis or inhibit enzymes for making proteins needed for reproduction of the cell. These work during the M phase of the cell cycle. Examples of mitotic inhibitors include paclitaxel, docetaxel, etoposide (VP-16), vinblastine, vincristine, and vinorelbine.

Steroids which are natural hormones and hormone-like drugs that are useful in treating some types of cancer (lymphoma, leukemias, and multiple myeloma) as well as other illnesses. When these drugs are used to kill cancer cells or slow their growth, they are considered chemotherapy drugs. They are often combined with other types of chemotherapy drugs to increase their effectiveness. Examples include prednisone and dexamethasone.

Sex hormones, or hormone-like drugs, which alter the action or production of female or male hormones. They are used to slow the growth of breast, prostate, and endometrial (lining of the uterus) cancers, which normally grow in response to hormone levels in the body. These hormones do not work in the same ways as standard chemotherapy drugs. Examples include anti-estrogens (tamoxifen, fulvestrant), aromatase inhibitors (anastrozole, letrozole), progestins (megestrol acetate), anti-androgens (bicalutamide, flutamide), and LHRH agonists (leuprolide, goserelin, histerillin, nafarelin, buserelin).

Alkylating agents which work directly on DNA to prevent the cancer cell from reproducing. As a class of drugs, these agents are not phase-specific (in other words, they work in all phases of the cell cycle). These drugs are active against chronic leukemias, non-Hodgkin's lymphoma, Hodgkin's disease, multiple myeloma, and certain cancers of the lung, breast, and ovary. Examples of alkylating agents include busulfan, chlorambucil, cyclophosphamide, ifosfamide, dacarbazine (DTIC), mechlorethamine (nitrogen mustard), and melphalan.

Platinum and other heavy metals may also be used in the treatment of cancers. Examples of platinum compounds and other compounds that may be used in the treatment of cancers include but are not limited to Cisplatin, oxaliplatin, carboplatin, diaminocyclohexylplatinin (DACH-platinum), nedaplatin, cycloplatan, SKI2053R, Loboplatin, enloplatin, zeniplatin, Miboplatin, L-NDDP, TRK-710, MBA (bis monobromo acetato trans 1,2-diaminocyclohexane platinum II), PYP (Bis-pyruvato 1,2-diaminocyclohexaneplatinum II), Sulphato 1,2 diaminocyclohexane platinum II, Malonato 1,2 diaminocyclohexane platinum II, Isocitrato 1,2-diaminocyclohexane platinum II, 4-Carboxyphtalato 1,2-diaminocyclohexane platinum II, bis-monobromoacetato trans 1,2-diaminocyclooctane platinum II, 1,1 diaminomethyl cyclohexane sulphate platinum II, JM-216 (bis-acetato-ammine-dichloro-cyclohexylamine platinum IV, Iproplatin (cis-dichloro-trans-dihydroxy-bis-isopropylamino platinum IV, CHIP, JM-9), Iproplatin (cis-dichloro-trans-dihydroxy-bis-isopropylamino platinum IV, CHIP, JM-9), 5-fluorouracil, leucovorin, Capecitabine, Gemcitabine, Topotecan, 9-aminocamtotecin, Rubitecan, Irinotecan, Exatecan, Docetaxol, Doxifluridine, Carmofur, UFT, Eniluracil, ZD-9331, MMI-166, Eniluracil, 6-hydroxymethylacylfulvene, Nediplatin, Satroplatin, Lipoplatin, BBR3464, ZD0473, lobaplatin, Spirogermanium, Gallium trinitrate, etoposide, gallium chloride, Budotitane, titanocene dichloride, trans-indazolium-[tetrachlorobis(2H-indazole)ruthenate(III,N1)] (HInd[RuInd2Cl4]), Trans-Na[RuInd2Cl4], ruthenium dimethylsulfoxide, thioguanidine, hexamethylmelamine), 5_nor-anhydro-vinblastine (Vinorelbine), Avastin (bevacizumab), BEC2 (mitomomab), Tositumomab (Bexxar),Campath (alemtuzumab), CeaVab, herceptin (trastuzumab), IMC-C225 (centuximab), Lymphocide (epratuzumab), MDX-210, Mylotarg (Gemtuzumab), Panorex (Edorcolomab), Rituxan (Rituximab), Theragyn (pemtumomab), Zamyl, Zevalin (Ibritumomab), Gefitinib (Iressa), Erlotinib, ABX-EGF, GSK 572016 (lapatinibditosylate), Cl-1033 (canertinib diHCl), Bortezomib, Arsenic trioxide (Trisenox), CC-394 (Actimid), CDC-501 (Revlimid), Thalidomide (Thalomid), Oblimersen (Genasense), Abarelix (Plenaxis), Zevalin, Gemtuzumab, 2-MPPA, CPI-0004Na.

Nitrogen mustard in the form of its crystalline hydrochloride which is used as a drug in the treatment of Hodgkin's disease, non-Hodgkin's lymphomas, and brain tumors. Nitrogen mustards cause mutations in the genetic material of cells, thereby disrupting mitosis, or cell division. Cells vary in their susceptibility to nitrogen mustards, with rapidly proliferating tumor and cancer cells most sensitive; bone marrow, which produces red blood cells, is also sensitive, and depression of red blood cell production is a frequent side effect of nitrogen mustard therapy. The nitrogen mustards also suppress the immune response (see immunity). Other types include the aromatic mustards melphalan and chlorambucil, cyclophosphamide, HN1, bis-(2-chloroethyl), ethylamine; HN2, bis-(2-chloroethyl), methylamine and HN3, tris-(2-chloroethyl), amine.

Nitrosoureas act in a similar way to alkylating agents. They interfere with enzymes that help repair DNA. These agents are able to travel to the brain so they are used to treat brain tumors as well as non-Hodgkin's lymphomas, multiple myeloma, and malignant melanoma. Examples of nitrosoureas include carmustine (BCNU) and lomustine (CCNU).

Hormone agonists such as Leuprolide (Lupron, Viadur, Eligard) for prostate cancer, Goserelin (Zoladex) for breast and prostate cancers and Triptorelin (Trelstar) for ovarian and prostate cancers and nafarelin acetate (Synarel).

Microtubule inhibitors include “Vinca” alkaloids, taxoids and benzimidazoles.

Curcuminoid derivatives such as tetrahydrocurcumin (THC), sodium circuminate, bisdemethoxycurcumin, demethoxycurcumin, 5′-methoxycurcumin and dihydrocurcumin, tetrahydrodemethoxycurcum in (INCI: tetrahydrodemethoxydiferuloymethane), and tetrahydrobisdemethoxycurcumin (INCI: tetrahydrobisdemethoxydiferuloylmethane.

Trichostatin A (TSA), an inhibitor of the enzyme deacetylase.

In one embodiment, the present invention contemplates a topical composition comprising ^{99m}Tc chelated to DTPA for the identification of a sentinel lymph node in relation to melanoma or breast carcinoma.

In an embodiment, the present invention contemplates a topical composition comprising one or more chemotherapeutic agents for the treatment of skin or breast cancer.

In another embodiment, the present invention contemplates a topical composition comprising a curcuminoid derivative for the treatment of melanoma or breast carcinoma.

In a related embodiment, the present invention contemplates a topical composition comprising TSA for the treatment of melanoma or breast carcinoma.

In yet another embodiment, the present invention contemplates a topical composition comprising an anthracyclin for the treatment of breast carcinoma. This composition may optimally also contain 5-fluorouracil and chlorambucil.

In yet a further embodiment, the present invention contemplates a topical composition comprising a chelate between an imaging agent and a chelating agent. Examples include, but are not limited to ^{99m}-Tc-HYNIC, ^{99m}-Tc-DTPA, ^{99m}Tc-ciprofloxacin, ^{99m}Tc-methylene diphosphonate (MDP), indium or technetium-^{99m}hexamethylpropylene amine oxime (HMPAO), ¹¹¹In-DTPA-Folate, ^{99m}Tc diadenosine tetraphosphate (Ap4A; AppppA, P1,P4-di(adenosine-5*)-tetraphosphate) and its analog ^{99m}Tc AppCHClppA, ^{99m}Tc-HYNIC-IL-8., ^{99m}Tc-TRODAT-1, ^{99m}Tc-M-TRODAT, ^{99m}Tc-RP128, Gd(III) DOTA, ^{99m}Tc-HYNIC-folate, [^{99m}Tc(SG38)(tricine)(TPPTS)] (RP517), natCu-DOTADY1-TATE and DOTA-natl-DY-TATE, ⁶¹Cu-DOTA-DY1-TATE, ^{99m}Tc labeled [(N-[2-((39-N9-propyl-[3,3,1]aza-bicyclononan-3a-yl)(20-methoxy-5-methyl-phenylcarbamate)(2-mercaptoethyl)amino)acetyl]-2-aminoethanethiolato] technetium(V) oxide), Albumin-(biotin)10-(gadopentetate)25, ^{99m}TcO(BAT-NI), (¹³¹1, ¹²³1, ′″In, (⁶⁷Ga, ⁶⁶Ga, ⁶⁴Cu, ⁶⁷Ga-deferoxamine-folate, ¹¹¹In-octadentate-DTPA-folate (g), ¹¹¹In-DTPA-NOON-pteroate., ^{99m}Tc(CO)3-DTPA-folate (g), ^{99m}Tc-EC20 (^{99m}Tc-Cys-Asp-Dap-D-Glu-Pte), ^{99m}Tc-oxa-PnAO-(a and g)-folate 1155, ^{99m}Tc-ethylene dicysteine (^{99m}Tc-L,L-EC), Co-⁵⁶, Co-⁵⁷, Co-⁵⁸, and Co-⁶⁰-labelled vitamin B12, DCTA, ¹¹¹Indium-DTPA-vitamin B12.

In a still further embodiment, the present invention contemplates a topical composition comprising a chemotherapeutic agent linked to a targeting agent. Such targeting agents selected from Amphiregulin, Antibodies including Avastin (bevacizumab), BEC2 (mitomomab), Tositumomab (Bexxar),Campath (alemtuzumab), CeaVab, herceptin (trastuzumab), IMC-C225 (centuximab), Lymphocide (epratuzumab), MDX-210, Mylotarg (Gemtuzumab), Panorex (Edorcolomab),Rituxan (Rituximab), Theragyn (pemtumomab), Zamyl, Zevalin (Ibritumomab), BAFF, BDNF, bFGF, BMP, BMP-4, BMP-7, CD209L, CSF, EGF, Fas-Ligand, Fc fragments, FGF, FGF2, FGFR1, FGFR4, Flt3, Flt3-Ligand, G-CSF, GM-CSF, hGH, hGHR, Growth Hormone Releasing Hormone and analogs, Growth Hormone Releasing Peptides, IFN, IgG, IGF-1, IGFBP-3, Interleukins, Langerin, LHRH analogs, LIF, L-Selectin, Lymphotoxin-a, MC-148, MCP-1, MIP-1a, MIP-1b, MLIF, NGF, NGFR, OSM, OX-40, PDGF, RANTES, Tf, TGF, TGF-b1, TNF, TNF-a, TPO, TrkA, TrkB, VEGF, Vitamin derivatives, including derivatives of vitamin B12, biotin, folate and/or riboflavin.

8. Skin Lightening and Re-Pigmentation.

The microemulsions of the present invention can be used to treat melasma and other areas where the skin contains no pigment or to lighten the skin where there areas of colored blotches. In one example, the microemulsions are used to introduce nucleic acid molecules to induce gene expression in order to produce pigment. Melanin and other pigmentation molecules may also be introduced.

Examples of skin lightening, skin whitening and re-pigmentation molecules include estrogen-like compounds such as chromenes, isoflavones and coumestran groups of phyloestrogens (coumestan glycosides). Examples of chromenes include microestrol and deoxymicroestrol. Examples of isoflavones include diadzen, genisten, mirificin, puerarin and puerarin-6″-monoacetate. Examples of coumestran glycosides include moumestrol, mirificoumestan, mirificoumestan glycol and mirificoumestran hydrate.

A useful source of estrogen-like compounds is from the plant genus Pueraria such as Pueraria lobata and Pueraria murifica and hence the present invention extends to the estrogen-like compound contained within an extract of a Pueraria plant such as an extract of P. murifica and/or P lobata.

Collagen fragment mimetics include dermaxyl (a palmitoyl oligopeptide), ceramide 2 (a natural skin lipid) and matrixyl 3000 which is a mixture of palmitoyl oligopeptides (e.g. palmitoyl-GHK and palmitoyl GQPR in a ratio of 2:1) may also be administered with the microemulsion alone or with an anti-oxidant.

Examples of suitable anti-oxidants include retinyl palmitate (Vitamin A), allantoin (also called 5-ureidohydantoin, glyoxyldiureide and 5-ureidohydantoin; it is a diureide of glycoxylic acid) and D-α tocopherol (Vitamin E).

9. Gene Therapy.

The microemulsions of the present invention are a useful vehicle to introduce nucleic acid molecules such as DNA, siRNA, anti-sense RNA, effector RNA molecules and microRNAs to inhibit gene expression or to facilitate gene expression. Reference to gene therapy in this regard includes the introduction of virosomes or constructs to facilitate gene expression. Gene therapy is useful inter alia to the treatment of cancer, diabetes and inflammatory conditions. In an embodiment, the present invention contemplates a topical composition comprising a siRNA for the treatment of cancer.

10. Muscle Dystrophy.

The microemulsions of the present invention are useful inter alia for delivering small molecules, proteins (including peptides and polypeptides and chimeras), antibodies, other water soluble molecules as well as oil-soluble molecules useful in the treatment of muscular dystrophy. An example of a small molecule with therapeutic potential in the treatment of muscular dystrophy is trichostatin A (TSA), an inhibitor of the enzyme deacetylase. In an embodiment, the present invention contemplates a topical composition comprising TSA for the treatment of muscular dystrophy.

11. Treatment of Neuropathic and Nociceptive Pain.

The microemulsions of the present invention may be used to introduce analgesic agents such as to include but not limited to fentanyl, oxycodone, codeine, dihydrocodeine, dihydrocodeinone enol acetate, morphine, desomorphine, apomorphine, diamorphine, pethidine, methadone, dextropropoxyphene, pentazocine, dextromoramide, oxymorphone, hydromorphone, dihydromorphine, noscapine, papverine, papveretum, alfentanil, buprenorphine and tramadol and pharmaceutically acceptable salts, derivatives, homologs or analogs thereof as well as opioid agonists. Both neuropathic and nociceptic pain may be treated and agents useful for either neuropathic or nociceptic pain may be introduced via the microemulsion. Pain includes the treatment of sport injuries, such as but not limited to, inflammatory conditions, planta fasciatis, bursitis of the knee, elbow, shoulder, subcutaneous prepatella bursa, olecranon bursa, tenosynovitis, Carpal Tunnel syndrome, bruising, Repetitive Strain Injury (RSI), patellotendonitis, paratendonitis, proliferative tendonitis, Degenerative tendonitis, enthesis and other conditions such as delayed onset muscle soreness (DOMS) and stings. In an embodiment, the present invention contemplates a topical composition comprising one or more analgesic agents for the treatment of neuropathic or nociceptic pain. In yet another embodiment of the invention the treatment of pain involves the use of a microemulsion containing a nitric oxide scavenger or an inhibitor of NOS such as Niacinamide, vitamin B12 and derivatives, curcumin and derivatives L-NAME,N^{G}-nitro-L-arginine methyl ester; L-NMA, N^{W}-methyl-L-arginine, L-NMMA, N^{G}-monomethyl-L-arginine, S-Methylisothiourea sulphate, Aminoguanidine. Inhibitor of iNOS, 7-nitro indazole or N-nitro-L-arginine.

12. Erectile Dysfunction.

The microemulsion of the present invention can be used to topically apply agents which are useful in treating erectile dysfunction including inhibitors of cGMP specific phosphodiesterases type 5 (PDE5). These molecules act by inhibiting the breakdown of nitric oxide (NO). Examples of PDE5 inhibitors include sildenafil, tadalafil and vardenafil. It is proposed that the microemulsion be topically applied to overcome erectile dysfunction. This approach avoids the side effects often associated with oral delivery of PDE5 inhibitors, including headache, lightheadedness, dizziness, flushing and change in vision. In a related embodiment, the present invention contemplates a topical composition comprising a PDE5 inhibitor for the treatment of erectile dysfunction.

Persistent, painful erection of the penis (priapism) is relatively common in young males with sickle cell anaemia but can also result following oral delivery of PDE5 inhibitors. It is therefore contemplated that the microemulsion of the present invention can be used to topically apply agents which enhance the break-down of NO for the treatment of persistent erection. Example of such molecules include NO chelators such as vitamin B12 derivatives as well as inhibitors of NO synthase (e.g. Niacinamide, curcumin and derivatives, L-NAME,N^{G}-nitro-L-arginine methyl ester; L-NMA, N^{W}-methyl-L-arginine, L-NMMA, N^{G}-monomethyl-L-arginine, S-Methylisothiourea sulphate, Aminoguanidine. 7-nitro indazole, N-nitro-L-arginine). In a preferred embodiment, the present invention contemplates a topical composition comprising an NO chelator for the treatment of persistent erection. In another embodiment, the present invention contemplates a topical composition comprising an NO synthase inhibitor for the treatment of persistent erection.

13. Acne Treatment.

The microemulsion of the present invention can be used to topically treat acne. Examples of agents which are useful in treating acne include curcumin, tetrahydrocurcumin, sodium circuminate, bisdemethoxycurcumin, demethoxycurcumin, 5′-methoxycurcumin and dihydrocurcumin, vitamin E, selenium, zinc, Vitamin C, azaleic acid, pantothenic acid, benzyl peroxide and extract of tea tree oil. In an embodiment, the present invention contemplates a topical composition comprising one or more compounds selected from the list of: Curcumin, tetrahydrocurcumin, sodium circuminate, bisdemethoxycurcumin, demethoxycurcumin, 5′-methoxycurcumin and dihydrocurcumin, vitamin E, selenium, zinc, vitamin C, azaleic acid, pantothenic acid, benzyl peroxide and extract of tea tree oil for the treatment of acne.

14. Treatment of Neurological Conditions.

The microemulsion of the present invention can be used to topically treat oxidative damage associated with neurological disease. Such diseases include neurodegenerative conditions like Parkinson's disease, Alzheimer's disease and other dementias as well as amyotrophic lateral sclerosis and multiple sclerosis. Other neurological diseases include those following acute injury such as stroke and spinal cord damage. Examples of agents which are useful in treating oxidative damage associated with neurological disease include NO chelators such as vitamin B12 derivatives as well as inhibitors of NO synthase (e.g. Niacinamide, vitamin B12 and derivatives, curcumin and derivatives L-NAME,N^{G}-nitro-L-arginine methyl ester; L-NMA, N^{W}-methyl-L-arginine, L-NMMA, N^{G}-monomethyl-L-arginine, S-Methylisothiourea sulphate, Aminoguanidine. 7-nitro indazole Inhibitro of nNOS, N-nitro-L-arginine). In a related embodiment, the present invention contemplates a topical composition comprising an NO synthase inhibitor for the treatment of acute CNS injuries such as stroke and spinal cord damage.

15. Treatment of Abrasions.

The microemulsion of the present invention can be used to topically treat skin damage associated with cuts and abrasions. Such skin damage is susceptible to microbial infection. Povidone-iodine type compounds such as Betadine are commonly used broad-spectrum topical microbicides as treatments for abrasions but can display poor capacity to penetrate within the wound. In an embodiment, the present invention contemplates a topical composition comprising a povidone-iodine type compound for the treatment of skin damage associated with cuts and abrasions.

The present invention provides, therefore, a microemulsion that is capable of transdermal delivery of pharmaceutical or physiologically useful or active agents. As used herein, the term “microemulsion” refers to a system comprising an oil phase, a water phase and a surfactant, wherein the microemulsion is a single optically isotropic and thermodynamically stable liquid solution. The term “transdermal delivery” is not to be construed as passage of an agent solely through the dermal layer although such passage is also contemplated by the present invention. The term provides passage of agents through all layers of the skin.

The microemulsion of the present invention comprises an oil phase, a water phase and a surfactant, wherein the microemulsion is capable of transdermal delivery of pharmaceutical agents. In one embodiment, the microemulsions further comprises a co-surfactant, a co-solvent, or a combination thereof.

The microemulsion of the present invention may be oil-in-water microemulsion, wherein the surfactant is preferentially soluble in water; water-in-oil microemulsion, wherein the surfactant is mainly in the oil phase; a three phase microemulsion wherein a surfactant rich middle phase coexists with water and oil phases; a bicontinuous monophase; a single phase micellar solution that forms upon addition of a sufficient quantity of amphiphile (surfactant plus alcohol); or a swollen micellar solution.

The microemulsion of the present invention may be produced by methods known in the art. In general, microemulsions are produced by emulsifying components under conditions including typically sufficient force or the required temperature to generate the required dispersion level, conductivity, viscosity, percolativity or other dispersion characteristics.

Microemulsion formation can be assessed using scattering and spectroscopic techniques such as neutron scattering, time-average scattering, quasi-electric light scattering i.e., high-resolution ultrasonic spectroscopy or photon correlation spectroscopy. The partition coefficients of microemulsions may also be measured chromatographically.

The selection of particular formulations is based on a number of different paradigms depending upon the desired application. Illustrative paradigms include the hydrophilic-lipophilic balance, the phase-inversion temperature, or the cohesive-energy ratio.

Microemulsions may be formulated using a wide range of immiscible liquids and other additional agents.

The microemulsion of the present invention may comprise an oil phase of between 50 and 99% by weight, most preferably between 50 and 90% by weight; a water phase of between 2 and 50% by weight, most preferably between 1 and 50 by weight; 0.1 to 90% by weight surfactant, preferably 1 to 90% by weight surfactant. The microemulsion may further comprise 0.1 to 90% by weight cosurfactant or cosolvent; preferably 1 to 90% by weight cosurfactant or cosolvent.

The oil phase may comprise natural oils derived from plants or animals, such as vegetable oils, sunflower oils, coconut oils, almond oils; purified synthetic or natural di or triglycerides (such as Crodamol GTCC™ and Capmul MCM™); phospholipids and their derivatives (such as lecithin or lysolecithin); fatty acid esters (such as isopropyl myristate, isopropyl palmitate, ethyl oleate, oleic acid ethyl ester); hydrocarbons (such as hexane, the n-decane through n-octadecane series); and/or glycerolysed fats and oils (such as glyceryl monooleate, glyceryl monocaprylate, glycerol monocaprate, propylene glycol monocaprylate, propyleme glycol monolaurate).

Other oil phase ingredients include, but are not limited to, Labrafil M 1944 CS™, benzene, tetrahydrofuran, and n-methyl pyrrolidone, or halogenated hydrocarbons, such as methylene chloride, or chloroform. In a particular embodiment, the oil phase comprises Crodamol GTCC™ and Capmul MCM™, at 3:1 ratio.

The oil component is either used alone or in combination with other oil component. Each oil or unique mixture of oils may require a different surfactant or mixture of surfactants or surfactants and co-surfactants to form a microemulsion with the water phase, as can routinely be determined by those of skill in the art.

Water phase ingredients may comprise water and any water-soluble components in water, including one or more pharmaceutical agent.

Surfactants used according to the present invention are known surfactants in the art that reduce the interfacial tension between the oil and water phases sufficiently to allow the formation of microemulsions in the subject compositions. Typically, surfactants are organic compounds that are amphiphatic, containing both hydrophobic groups and hydrophilic groups. Surfactants include, but are not limited to anionic surfactants, cationic surfactants and non-ionic surfactants.

Anionic surfactants include fatty acid soaps (including sodium oleate, sodium palmitate, sodium myristate, sodium sterate, potassium oleate and triethanolamine oleate); alkyl sulfates (including sodium dodecyl sulfate, ammonium lauryl sulfate, triethanolamine lauryl sulfate and sodium alkyl sulfate); alkyl lactylates (including calcium stearoxyl-2-lactylate), alkyl lactates (including sodium-O-stearyllactate and sodium stearoyllactylate) alkyl benzenesulfonates (including calcium dodecyl benzene sulfonate); alkyl sulfonates (including alkyl aryl sulfonate); alkyl phosphates; alkyl oleates; alkyl stearates (including self-emulsifying glycerol monostearate); alkyl esters (including dioctyl ester of sodium sulphosuccininc acid (AOT, Aerosol OT)); acyl sulfates; or acyl sulfosuccinates.

Cationic surfactants include alkyl primary, secondary, tertiary, or quaternary amines; high-molecular-weight amine and fatty amine blends; polyoxyethylene fatty amines (including tallow amine); alkyl sulfates (including N-cetyl-N-ethyl morpholinium ethyl sulfate(35%)); alkyl pyridinium and quaternary ammonium salts.

Non-ionic surfactants include alcohol ethoxylate, alkylphenol ethoxylate, fatty acids (such as oleic acid), lanolin alcohols (such as polyoxyethylene (5) lanolin alcohol (ether and ester), polyoxyethylene (50) lanolin (ether and ester), acetylated polyoxyethylene (10) lanolin, polyoxyethylene (16) lanolin alcohol, acetylated polyoxyethylene (9) lanolin), alkyl polyglycosides, mono-, di- or glyceride esters (such as diglycerine sesquioleate), acetylated monoglycerides, polyglycerols, polyglycerol esters (such as decaglycerol decaoleate, decaglycerol octaoleate, decaglycerol tetraoleate), phospholipids (such as lecithin), mono- or diglyceride esters of citric acid, tartaric acid and lactic acid, sorbitan fatty acid esters (such as sorbitan monostearate (Span 60, Crill 3), sorbitan monooleate (Arlacel 80, Span 80, Crill 4), sorbitan isosterate (Crill 6), sorbitan monolaurate (Arlacel 20, Span 20, Crill 1), sorbitan trioleate (Span 85, Crill 45), sorbitan tristearate (Span 65), sorbitan sesquioleate (Arlacel 83, Crill 43), sorbitan monopalmitate (Span 40, Crill 2)), polyol fatty acid esters (such as ethylene glycol distearate, ethylene glycol monostearate, diethylene glycol monostearate, propylene glycol monostearate, propylene glycol monolaurate, polyoxyethylene (1.5) nonylphenol, polyoxyethylene (4) nonylphenol, polyoxyethylene (5) nonylphenol, polyoxyethylene (6) nonylphenol, polyoxyethylene (8) nonylphenol, polyoxyethylene (20) nonylphenol, polyoxyethylene (30) nonylphenol, polyoxyethylene (10) nonylphenol, poly(ethylene glycol) 200 distearate, poly(ethylene glycol) 300 dilaurate, poly(ethylene glycol) 400 distearate, polyoxyethylene octylphenol, poly(ethylene glycol) 400 dilaurate, poly(ethylene glycol) 400 monostearate, poly(ethylene glycol) 400 monolaurate, poly(ethylene glycol) 4000 distearate, polyoxyethylene (10) octylphenol, poly(ethylene glycol) 600 monostearate, Polyoxyethylene (14) nonylphenol, polyoxyethylene (24) cholesterol, polyoxyethylene (25) soyasterol, poly(ethylene glycol) 1000 monooleate, polyoxyethylene (25) propylene glycol monostearate, poly(ethylene glycol) 1000 monolaurate, polyoxyethylene (70) dinonylphenol), glycerol fatty acid esters (such as glycerol dioleate, glycerol monoleate, glycerol monostearate, glycerol monolaurate, polyoxyethylene (20) glycerol monostearate), sucrose fatty acid esters (such as sucrose distearate, sucrose monolaurate), polyoxyethylene sorbitan fatty acid esters (polysorbates) (such as polyoxyethylene (4) sorbitan monolaurate, polyoxyethylene (5) sorbitan monooleate, polyoxyethylene (20) sorbitan monooleate (Tween 80), polyoxyethylene (40) sorbitol hexaoleate, polyoxyethylene (50) sorbitol hexaoleate, polyoxyethylene (20) sorbitan tristearate, polyoxyethylene (20) sorbitan trioleate, polyoxyethylene (20) sorbitan monostearate, polyoxyethylene (20) sorbitan monopalmitate, polyoxyethylene (20) sorbitan monolaurate (Tween 20), polysorbate 20 NF, EP, JP, poly(ethylene glycol)-20 sorbitan isostearate, poly(ethylene glycol) (20) sorbitan trioleate (Crillet 45), poly(ethylene glycol) (20) sorbitan stearate (Crillet 3 Super, Polysorbate 60), poly(ethylene glycol) (20) sorbitan oleate (Crillet 4 Super, Polysorbate 80), poly(ethylene glycol) (20) sorbitan laurate (Crillet 2 Super, Polysorbate 40)), monoesters (such as polyoxyethylene (4) stearic acid, polyoxyethylene (8) stearic acid, polyoxyethylene (8) lauric acid, polyoxyethylene (40) stearic acid, polyoxyethylene (50) stearic acid), polyethoxylated esters of acyl acids (such as polyoxyethylene (2) octyl alcohol, polyoxyethylene (4) tridecyl alcohol, polyoxyethylene (6) tridecyl alcohol, polyoxyethylene (8) tridecyl alcohol), copolymers of polyethylene oxide and polypropylene oxide, polyoxyethylene fatty ethers (such as polyoxyethylene fatty ethers derived from lauryl, cetyl, stearyl and oleyl alcohols, polyoxyethylene (4) lauryl ether, polyoxyethylene (23) lauryl ether (Brij 35), polyoxyethylene (2) cetyl ether (Brij 52), polyoxyethylene (10) cetyl ether, polyoxyethylene (20) cetyl ether (Brij 58), polyoxyethylene (2) stearyl ether (Brij 72), polyoxyethylene (10) stearyl ether, polyoxyethylene (20) stearyl ether, polyoxyethylene (2) oleyl ether, polyoxyethylene (10) oleyl ether (Brij 97), polyoxyethylene (20) oleyl ether, polyoxyethylene (21) stearyl ether, polyoxyethylene (12) lauryl ether), fatty amides (such as N,N,-Dimethylstearamide), Polyethylene glycol ether of linear alcohol, polyoxyethylene (15) tall oil fatty acids (ester), acetylated sucrose diesters, isopropyl ester of lanolin fatty acids, polyoxyethylene sorbitol beeswax derivative, Polyoxypropylene/Polyoxyethylene condensate, sodium oleate, polyoxyethylene (20) castor oil (ether, ester), glycerol oleate & propylene glycol (Arlacel 186) and Cremophor.

Surfactants in the microemulsion of the present invention may be used alone or in combination with each other.

Co-surfactants used according to the subject invention are known surface-active agents in the art that act, in addition to surfactants, to further lower the interfacial energy of a microemulsion. Co-surfactants include, but are not limited to non-toxic amphiphilic molecules; alcohols (including aliphatic alcohols, shorter chain alcohols, such as ethanol); fatty acid alcohols (such as n-alkane-1,2-diols); acids (such as acetic acid); esters (such as butyl lactate); any surfactants as herein listed or mixtures thereof. Alcohol content may range, for example, from 0 to about 30% by volume in the microemulsion.

The microemulsion of the present invention may further comprise solvents or other agents to enhance emulsion formation or stability. Other agents may be introduced to provide functions such as pH, ionic content, polymerisation, taste, smell, sterility, colour, viscosity etc.

The microemulsions of the present invention may also be generated using any suitable synthetic plastic or polymeric, monomeric or hybrid colloidal material.

The present invention further extends to a pharmaceutical composition comprising the microemulsion and one of more pharmaceutical agents as described herein, wherein such pharmaceutical composition is suitable for topical application on a subject.

Suitable pharmaceutical agents for use in the present invention include any water soluble solute, including, but not limited to peptides, proteins, polysaccharides, oligonucleotides, salts, sugars, nutrients, vitamins, nucleic acids minerals, acids, anti-oxidants, or any biological active compounds for administration to a subject, such as a human, animal or other mammal. The pharmaceutical agent is selected based upon the intended application or therapy, wherein the effect of the pharmaceutical agent is suitable to treat a particular condition.

Suitable peptides and proteins that can be delivered via the microemulsion of the present invention include molecules, protein chimeric molecules, other chimeric molecules or fragments selected from the TNF superfamily (including TNF-α, TNFR1, TNFR2, BAFF, OX-40, Lymphotoxin-α, Fas-ligand); chemokines (including MCP-1, MIP-1a, MIP-1b, RANTES, IL-8 and viral like chemokine antagonist MC148); interleukins, interleukin receptors and antagonist (including IL-1a IL-1b, IL-2, IL-3, IL-4, IL-5, IL-6, IL-7, IL-8, IL-10, IL-11, IL-12, IL-13, IL-15, IL-16, IL-18, IL-20, IL-21, IL-22, IL-23, IL-24, IL-25, IL-26, IL-27, IL-28, IL-29, IL-30, their respective receptors including IL-1Ra, IL-2Ra, IL-2Rb, IL-2Rg, IL-3Ra, IL-4Ra, IL-5Ra, IL-6Ra, IL-7Ra, IL-10Ra, IL-11Ra, IL-13Ra, IL-15Ra as well as IL-1R Antagonist); the interferon family (including IFN-a2B, IFN-b1, IFN-g, IFN-y, IFN-aR2, IFN-aRa, IFN-aRb, IFN-gRa, IFN-gRb); lectins (including CD209 type I and II, E-Selectin, L-Selectin, P-Selectin, Langerin); growth factors and their receptors (including Amphiregulin, Angiopoietin, BDNF, beta-cellulin, BMPs (including BMP-2, BMP-4, BMP-7), CNTF, cripto, ECGF-1, EGF, EGFR, EPO, FGFs and their receptors (including FGF-1, FGF-2, FGF-5, FGF-7, FGF-9, FGF-11, FGF-12, FGF-13, FGF-14, FGF-14 FLAG, FGF-18, FGF-19, FGF-21, FGFR1, FGFR1, FGFR4, FGFR5), Flt3-Ligand and its receptor (including Flt3), G-CSF, GDNF, GM-CSF, GM-CSF-R, hGH and its receptor (including hGHR), IGF-1, IGFBP-3, M-CSF, Neuregulin, NGFs and its receptor (including NGF-b, NGFR, NGFR), NT-3, PDGFS, TGFs and their receptors (including TGF-a, TGF-b, TGFbR2), Trk-A, Trk-B , TPO, VEGFs and their receptors (including VEGF-A, VEGF-B, VEGF-C, VEGF-D, VEGF-165, VEGFR); embryonic growth factors (such as Noggin, Nodal, SCF, Wnts, Wnt-2, Wnt-3, Wnt-3A, Wnt-4, Wnt-5A, Wnt-5A-FLAG-C, Wnt-5B, Wnt-6, Wnt-7A, Wnt-7B, Wnt-10A, Wnt-10B, Wnt-10B-FLAG-C, Wnt-11); adhesion molecules (such as adiponectin, ICAM), other cytokines and proteins, such as LIF, OSM, transferring and its receptor, hormones (such as insulin, calcitonin, adrenocorticotropin (ACTH), glucagon, somatostatin, somatotropin, thymosin, parathyroid hormone, pigmentary hormones, somatomedin, lutenizing hormone and agonists and antagonists thereof, chorionic gonadotropin, hypothalmic releasing factors, antidiuretic hormones, vasopressin, thyroid stimulating hormone, endorphins, enkephalins, biphalin and prolactin.); antibiotics (such as gentamycin, amikacin, neomycin, penicillin, streptomycin), enzymes (such as activin A asparaginase, adenosine deaminase, BACE-1, caspase-1, fucosyltransferase, furin, mTACE, sialyltransferase) Factor VIII, LH-RH analogs, anticoagulants such as Heparin, Warfarin, Herbal extracts such as those derived from Danshen Devil's Claw, Eleuthero, Garlic, Ginger, Ginkgo, Horse Chestnut, Panax Ginseng, Papain, Red Clover, Saw Palmetto, capsaicin and vaccines (for instance, vaccines for Hepatitis ‘B’ surface antigen, typhoid and cholera vaccines) and plasminogen activator inhibitors, and small peptides such as MLIF (Met-Gln-Cys-Asn-Ser), or analogs thereof.

In particular, suitable chimeric proteins that can be delivered via the microemulsion of the present invention include TNFR1-Fc, TNFR2-Fc, OX-40-Fc, MC148-Fc, IL-1Ra-Fc, IL-2Ra-Fc, IL-2Rb-Fc, IL-2Rg-Fc, IL-3Ra-Fc, IL-4Ra-Fc, IL-5Ra-Fc, IL-6Ra-Fc, IL-7Ra-Fc, IL-10Ra-Fc, IL-11Ra-Fc, IL-13Ra-Fc, IL-15Ra-Fc, IFN-aRa-Fc, IFN-aRb-Fc, IFN-gRa-Fc, IFN-gRb-Fc, CD209L-Fc, E-Selectin-Fc, L-Selectin-Fc, P-Selectin-Fc, Langerin-FLAG, EGFR-Fc, FGFR1-Fc, FGFR4-Fc, FGFR5-Fc, Flt3-Fc, hGHR-Fc, NGFR-Fc, TGFbR2-Fc, Trk-A-Fc, Trk-B-Fc, VEGFR-Fc, Wnt-5A-FLAG-C, Wnt-10B-FLAG-C and, most preferably, TNFR1-Fc, TNFR2-Fc.

In another embodiment, the pharmaceutical agent of the present invention comprises MLIF or analogs thereof, such as iodacetylated MLIF.

Additional suitable proteins include monoclonal and polyclonal antibodies, single-chain antibodies, other antibody fragments, analogs and derivatives thereof. Polynucleotides, including antisense oligonucleotides, aptamers and therapeutic genes can also be delivered using the methods and compositions of the present invention.

In a further embodiment, suitable antibodies that can be delivered via the microemulsion of the present invention include antibodies directed against TNF alpha, IL-1, IL-8, IL-6 or other cytokines involved in the inflammatory response and, most preferably, anti-TNF alpha antibodies with the technical names of infliximab and adalimumab.

Anticoagulants, such as Heparin, Warfarin, Herbal extracts such as those derived from Danshen Devil's Claw, Eleuthero, Garlic, Ginger, Ginkgo, Horse Chestnut, Panax Ginseng, Papain, Red Clover, Saw Palmetto, capsaicin, also can be delivered using the methods and compositions of the invention. Still other suitable therapeutic agents for use in the present invention include bioactive molecules, such as anticancer drugs, e.g., Curcumin derivatives, Glycyrrhizin, Glycyrrhetinic acid, antracycline, platinum drugs, methotrexate derivatives, heary metals, genistein, chlorambucil, cyclophosphamide, melphalan, cyclopropane, doxorubicin, daunomycin, adriamycin, mitomycin C, [2-(hydroxymethyl)anthraquinone], methotrexate, dichloromethatrexate: cisplatin, carboplatin, metallopeptides containing platinum, copper, vanadium, iron, cobalt, gold, cadmium, zinc and nickel, DON, thymidine, pentamethylmelamin, dianhydrogalactitol, 5-Methyl-THF, anguidine, maytansine, neocarzinostatin, chlorozotocin, AZQ, 2′deoxycoformycin, PALA, AD-32, m-AMSA and misonidazole, doxorubicin, epirubicin and daunorubicin, vincristine, vinblastin methotrexate, paclitaxol, taxol, camptothecin and camptothecin analogs, antipsychotics, antidepressants, and drugs for diabetes and cardiovascular disease.

Other pharmaceutical agents that can be delivered via the microemusltion of the present invention are ones which have anti-inflammatory properties, such as NSAIDs. Exemplary NSAIDs include salicylates (including aspirin, choline magnesium trisalicylate, dilfunisal, salasalate, benorylate); phenylalkanoic acids (including carpofen, fenoprofen, fluribiprofen, ibuprofen, ketoprofen, naproxen, naproxen Na, suprofen, oxaprozin,); acetic acids or indoles (including alclofenac, diclofenac, fenclofenac, indomethacin, sulindac, tolemetin); enolic acids (including isoxicam, meloxicam, piroxicam, tenoxicam); fenamic acids (including flufenamic acid, meclofenamate, mefenamic acid); napthylalkanones (including nabumetone); niflumic acid; pyranocarboxylic acids (including etodolan); pyrazolones (including phenylbutazone, oxyphenobutazone); pyrroles (including ketorolac); COX-2 inhibitors (including celecoxib, parecoxib, valdecoxib, rofecoxib). Some brand names of NSAIDs are shown in Table 2.

TABLE 2
Brand Names of NSAIDs

| | |
|---|---|
| Generic Name | Brand Name |

Salicyclic acids

| | |
|---|---|
| Aspirin (acetylsalicyclic acid) | Ascriptin, Bayer, Ecotrin |
| Choline magnesium trisalicylate | Trilisate |
| Diflunisal | Dolobid |
| Salsalate | Disalcid, Salflex |

Phenylalkanoic acids

| | |
|---|---|
| Fenoprofen | Nalfon |
| Flubiprofen | Ansaid |
| Ibuprofen | Advil, Motrin, Nuprin |
| Ketoprofen | Actron, Orudis, Oruvail |
| Naproxen | Aleve, Anaprox, Naprelan, Naprosyn |
| Naproxen Na | Anaprox |
| Suprofen | Suprol |
| Oxaprozin | Daypro |

Acetic Acids/Indoles

| | |
|---|---|
| Diclofenac | Cataflam, Voltaren |
| Indomethacin | Indocin |
| Sulindac | Clinoril |
| Tolmetin | Tolectin |

Enolic acids

| | |
|---|---|
| Meloxicam | Mobic |
| Piroxicam | Feldene, Fexicam |

Fenamic acids

| | |
|---|---|
| Meclofenamate | Meclomen |
| Mefenamic acid | Ponstel |

Naphthylalkanones

| | |
|---|---|
| Nebumetone | Relafen |

Pyranocarboxylic acids

| | |
|---|---|
| Etodalac | Lodine |

Pyrroles

| | |
|---|---|
| Ketorolac | Toradal |

COX-2 inhibitors

| | |
|---|---|
| Celecoxib | Celebrex |
| Valdecoxib | Bextra |
| Rofecoxib | Vioxx |

In another embodiment, the pharmaceutical agent suitable to be delivered via the microemulsion of the present invention comprises copper complexes of NSAIDs. Formation of copper complexes of NSAIDs reduces toxicity and may enhance the anti-inflammatory properties of the complex due to the anti-inflammatory property of copper. Such pharmaceutical agents include copper complexes of salicylates (including aspirin, choline magnesium trisalicylate, dilfunisal, salasalate, benorylate); copper complexes of phenylalkanoic acids (including carpofen, fenoprofen, fluribiprofen, ibuprofen, ketoprofen, naproxen, naproxen Na, suprofen, oxaprozin); copper complexes of acetic acids or indoles (including alclofenac, diclofenac, fenclofenac, indomthacin, sulindac, tolemetin); copper complexes of enolic acids (including isoxicam, meloxicam, piroxicam, tenoxicam); copper complexes of fenamic acids (including flufenamic acid, meclofenamate, mefenamic acid); copper complexes of napthylalkanones (including nabumetone); copper complexes of niflumic acid; copper complexes of pyranocarboxylic acids (including etodolan); copper complexes of pyrazolones (including phenylbutazone, oxyphenobutazone); copper complexes of pyrroles (including ketorolac); copper complexes of COX-2 inhibitors (including celecoxib, parecoxib, valdecoxib, rofecoxib).

In yet another embodiment, the pharmaceutical agent may be selected from copper complexes of tyrosine, lysine, valproic acid, 2,2,-bypyridine, 1,10-phenanthroline, 2,9-dimethyl-1,10-phenanthroline, glycylglycine, cimetidine, sulfathiazole, tetraanhydroaminmobenzaldehyde, metronidazole and imidazole.

Other potential complexes suitable for delivery as pharmaceutical agents include copper complexes with amino acids, aromatic carboxylic acid, corticoids, tetrazoles, histamines, penicillamines, anti-ulcer histamine antagonists, rantidine and cimetidine.

The aforementioned copper complexes may further comprise one of more molecules of DMF, DMSO, Pyridine, Caffeine, Methylimidazole, Imidazole, 2-methylbenzimidazole, metronidazole, 2-methylimidazole, 3-picoline, 4-picoline, imidazole, 1-methylimidazole, diethylamine, nicotinamide, papaverine, N,N-dimethylacetamide, N-methyl-2-pyrrolidone, ethanol, methanol.

The pharmaceutical composition of the present invention comprising one or more copper-NSAID complexes may be formed by mixing two microemulsions of the present invention containing NSAID and copper respectively.

Suitable vitamins include but are not limited to water soluble vitamins such as vitamin C, analogs or derivatives thereof; folic acid and analogs or derivatives thereof (including but not limited to methotrexate, aminopterin, 10-deazaminopterin, 10-ethyl-10-deazaaminopterin, 5,10-dideazatetrahydrofolate, folinic acid, 7-hydroxyaminopterin); niacin (nicotinic acid, vitamin B3) and analogs or derivatives thereof (including but not limited to beta-hydroxybutyrate, acipimox, niceritrol, nicotinamide (niacin)); thiamine (vitamin B-1), analogs and derivatives thereof; riboflavin (vitamin B2) and its analogs or derivatives thereof (including but not limited to 7-nor-7-chlororiboflavin, 8-nor-8-chlororiboflavin, 7-nor-7-bromoriboflavin, 8-nor-8-bromoriboflavin, 7-methylriboflavin, 8-methylriboflavin, 7,8-dimethylriboflavin, 7-nor-7-bromo-8-methylriboflavin, 7-methyl-8-nor-8-bromoriboflavin, 7-nor-7-chloro-8-methylriboflavin, 7-methyl-8-nor-8-chlororiboflavin, 8-nor-8-fluororiboflavin, 7-nor-7-chloro-8-nor-8-chlororiboflavin, 8-nor-8-aminoriboflavin, N(3)-methylriboflavin and 5-deaza-5-carbariboflavin); pyridoxine (vitamin B6), analogs or derivatives thereof; cyanocobalamin (vitamin B12), analogs or derivatives thereof; pantothenic acid (vitamin B5), analogs or derivatices thereof; biotin, analogs or derivatives thereof; and the vitamin E derivative Trolox. Suitable vitamins also include but are not limited to oil soluble vitamins such as vitamin A, analogs or derivatives thereof, such as acitretin (Soriatane); vitamin E (alpha tocopherol) analogs or derivatives thereof.

Suitable vitamin B12 (VB12) analogs according to the invention include descobaltocorrinoids such as alpha (5,-6-dimethylbenzymidazolyl)-hydrogenobamide, hydroxocobalamin (OH-Cbl), methylcobalamin, adenosylcobalamin (AdeCbl), aquocobalamin, methylcobalamin, cyanocobalamin, carbanalide, 5 methoxybenzylcyanocobalamin [(5-MeO)CN-Cbl], as well as the desdimethyl, monoethylamide and the methylamide analogs of all of the above VB12 molecules. Other suitable VB12 analogs include all alkyl cobalamins in which the alkyl chain is linked to the corrin nucleus by a direct CoC covalent bond. Still other suitable VB12 analogs include chlorocobalamin, sulfitocobalamin, nitrocoba lamin, thiocyanatocobalamin, benzimidazolecyanocobalamin derivatives such as 5,6 dichlorobenzimidazole, 5-hydroxybenzimidazole, trimethylbenzimidazole, as well as adenosylcyanocobalamin [(Ade)CN-Cbl], cobalamin lactone, cobalamin lactam and the anilide, ethylamide, monocarboxylic and dicarboxylic acid derivatives of VB12 or its analogs. Other VB12 derivatives include adeninylalkylcobalamin, adeninylethylcobalamin (AdeEtCbl), adeninylpropylcobalamin (AdePrCbl) and adeninylpentylcobalamin (AdePeCbl)). Alternative derivatives of VB12 include the mono, di- and tricarboxylic acid derivatives or the propionamide derivatives of VB12. Alternatively, suitable analogs of VB12 include those in which the cobalt ion is replaced by either a zinc or nickel ion. Other derivatives and analogs of vitamin B12 are discussed in Schneider and Stroinski, Comprehensive B12, Walter De Gruyter, Berlin, N.Y., 1987, the disclosure of which is incorporated herein by reference.

In a preferred embodiment, the pharmaceutical agent of the present invention comprises aquocobalamin, adenosylcobalamin, methylcobalamin, hydroxycobalamin, cyanocobalamin, carbanalide, or 5 methoxybenzylcyanocobalamin [(5-MeO)CN-Cbl].

Without limiting the scope of the invention, VB12 analogs or derivatives of the present invention are involved in the sequestration and/or neutralization of nitric oxide and other reactive nitrogen species. Thus, nitric oxide and nitric superoxide rapidly combine to form a toxic reaction product, peroxynitrite anion (ONOO). The ratio of superoxide to NO is important in determining the reactivity of peroxynitrite: excess NO or excess superoxide reduces the oxidation elicited by peroxynitrite. The oxidant reactivity of peroxynitrite is mediated by an intermediate with the biological activity of the hydroxyl radical. Activated macrophages have been shown to secrete high levels of NO due to inducible NO synthase (iNOS). Molecules such as vitamin B12 have been shown to reduce the activity of NO by complexation to the CN group. Additionally, the skin contains many dendritic cells, which have also been shown to produce iNOS when stimulated. Such stimulation may occur in clinical conditions such as psoriasis.

Suitable curcumin analogs include tetrahydrocurcumin, bisdemethoxycircumin, extracts from Curcuma xanthorrhiza or C. domestica such as 25 P54FP and extracts from C. phaeocaulis valeton. Suitable curcumin derivatives include 5′-methoxycurcumin, dihydrocurcumin, cyclocurcumin and demethoxy curcumin, as well as curcumins and curcuminoids such as those described in the Chinese Pharmacopoeia; Yujin (from the tubers of C. wenyujin, C. longa, C. kwangsiensis or C. phaeocaulis); Jianghuang (the rhizome of C. longa); Pian-Jianghuang (the rhizome of C. wenyujin) and Ezhu (the rhizomes of C. phaeocaulis, C. kwangsiensis or C. wenyujin) as well as in the rhizomes of C. zedoaria and C. aromatica. Additional molecules with anti-inflammatory properties include those that are structurally related to curcumin, such as diarylheptanoids obtained from Alpinia oxyphylla, several sesquiterpenes, germacrone, turmerone, ar-(+)-, a-, β-turmerones; β-bisabolene; a-curcumene; zingiberene; β-sesquiphellandene, bisacurone; curcumenone; dehydrocurdione; procurcumadiol; bis-acumol; curcumenol; isoprocurcumenol epiprocurcumenol; procurcumenol; zedoaronediol; curlone; and turmeronol A and turmeronol B, as well as four polysaccharides-ukonans—having activity on the Reticuloendothelial system, along with stigmasterol, β-sitosterol, cholesterol and 2-hydroxymethyl anthraquinone.

In a further embodiment, the pharmaceutical agent of the present invention comprises a curcumin analog and, in particular, tetrahydrocurcumin.

In a particular embodiment, the pharmaceutical composition of the present invention comprises a microemulsion with an oil phase composed of mixtures of oil, in particular Crodamol GTCC™ (caprylic/capric triglyceride) and Capmul MCM™ (medium chain mono- and diglyceride), at 3:1 ratio; surfactants and co-surfactants Crillet 4™ (polysorbate 80) and Crill 4™ (sorbitan monooleate) at 3:2 ratio, with one ninth volume of water containing one or more pharmaceutical agents in solution. The concentration of the pharmaceutical agent may be between 0.1 μg/ml to 100 mg/ml.

In another particular embodiment, the pharmaceutical composition of the present invention comprises a microemulsion including 3.5 ml of hexane and 1.5 g surfactants and co-surfactants Crillet 4™ (polysorbate 80) and Crill 4™ (sorbitan monooleate) at 1:1 ratio; 260 μl of solution with one or more pharmaceutical agents at 10 mg/ml.

In an additional embodiment, the pharmaceutical composition of the present invention comprises a base stable microemulsion including 16 g of oil components (Crodamol GTCC™ and Capmul™, at 3:1 ratio), 4 g of surfactants and co-surfactants Brij 72 (polyoxyethylene (2) stearyl ether) and Brij 97 (polyoxyethylene (10) oleyl ether) at a ratio of 3:1, and 0.5 ml of protein solution (2-10 mg/ml).

In a further embodiment, the pharmaceutical composition of the present invention comprises a microemulsion, an emulsion or a cream and one of more pharmaceutical agent as described herein, wherein such pharmaceutical composition is suitable for topical application on a subject.

The present invention further provides a method suitable for the transdermal delivery of pharmaceutical agents in a range of therapeutic, cosmetic and research applications. The method comprises firstly preparing a water phase, wherein any water-soluble components, including any pharmaceutical agents, are dissolved in water. Oil soluble components are dissolved in the oil phase as appropriate. Any undissolved material is separated by centrifugation or filtration. The oil and water phases and one or more suitable surfactants or co-surfactants are then mixed in a suitable vessel, and given time to equilibrate. Standard emulsification techniques, like stirring, use of membranes, applying shear, ultrasound and the like, can be used to facilitate the mixing of the two phases. The pharmaceutical composition is then applied on a subject.

Examples of therapeutic, cosmetic and research applications of the present invention include any skin conditions, or any dermatologic disorders, including dermatitis, bacterial, fungal, parasitic, viral infections of the skin, disorders of hair follicles and sebaceous glands, scaling papular diseases, psoriasis, inflammatory reactions, reaction to sunlight, bullous diseases, disorders of cornification, pressure sores, pigmentation disorders, disorders of sweating, benign and malignant tumours.

Examples of dermatitis include contact dermatitis, atopic dermatitis, seborrheic dermatitis, nummular dermatitis, chronic dermatitis of hands and feet, generalized exfoliative dermatitis, stasis dermatitis, lichen simplex chronicus.

Examples of bacterial infections of the skin include cellulitis, acute lymphangitis, lymphadenitis, erysipelas, cutaneous abscesses, necrotizing subcutaneous infections, staphylococcal scalded skin syndrome, folliculitis, furuncles, hidrandenitis suppurativa, carbuncles, paronychial infections, erythrasma.

Examples of fungal skin infections include dermatophyte infections including tinea corporis, tinea pedis, tinea unguium, tinea capitis, tinea cruris, tinea barbae, dermatiphytids or Id Eruptions; yeast infections including candidiasis, tinea versicolor.

Examples of parasitic skin infections include scabies, pediculosis, creeping eruption.

Examples of viral skin infections include warts and molluscum contagiosum.

Examples of hair follicles and sebaceous glands disorders include acne, rosacea, perioral dermatitis, hypertrichosis, alopecia, pseudofolliculitis barbae, keratinous cyst.

Examples of scaling papular diseases include psoriasis, pityriasis rosea, lichen planus, pityriasis rubra pilaris.

Examples of inflammatory reactions of the skin include drug eruptions, toxic epidermal necrolysis, erythema multiforme, erythema nodosum, granuloma annulare.

Examples of skin reaction to sunlight include sunburn, chronic effects of sunlight, such as actinic keratoses, squamous and basal cell carcinoma and malignant melanomas, and photosensitivity.

Examples of bullous diseases include pemphigus, bullous pemphigoid, dermatitis herpetiformis, linear immunoglobulin A disease.

Examples of cornification disorders include ichthyosis, keratosus pilaris, calluses and corns.

Pressure sores results from ischemic necrosis and ulceration of tissues overlying a bony prominence that has been subjected to prolonged pressure against an external object, such as bed, wheelchair, cast or splint. Examples of pressure sores include bedsores, decubitus ulcers, trophic ulcers.

Examples of pigmentation disorders include hypopigmentation and hyperpigmentation.

Examples of sweating disorders include miliaria and hyperhidrosis.

Examples of benign tumours include moles, dysplastic nevi, skin tags, lipomas, angiomas, pyogenic granuloma, seborrheic keratoses, dermatofibroma, keratoacanthoma, keloid.

Examples of malignant tumours include basal cell carcinoma, squamous cell carcinoma, Bowen's disease, malignant melanoma, breast cancer, Paget's disease of the nipples, and Kaposi's sarcoma.

Other examples of therapeutic, cosmetic and research applications of the present invention include any musculoskeletal and connective tissue disorders, including rheumatoid arthritis, Sjogren's syndrome, Behcet's syndrome, relapsing polychondritis, systemic lupus erythematosus, discoid lupus erythematousis, systemic sclerosis, eosinophilic fasciitis, polymyositis, dermatomyositis, polymyalgia rheumatica, vasculitis, termporal arteritis, polyarteritis nodosa, Wegener's granulomatosis, mixed connective tissue disease, ankylosing spondylitis, Reiter's syndrome, psoriatic arthritis, osteoarthritis, neurogenic arthropathy, avascular necrosis, infectious arthritis, ostemyelitis, gout, idiopathic hyperuricemia, calcium pyrophosphate dihydrate crystal deposition disease, basic calcium phosphate and other crystal disorders, muscular dystrophies, benign and malignant tumors of bone, osteoporosis, Paget's disease of bone, nonarticular rheumatism (such as spasmodic torticollis, low back pain, bursitis, tendinitis, tenosynovitis, fibromyaglia, ankle sprains, disorders associated with heel pain (such as calcaneal spur syndrome, epiphysitis of the calcaneus, posterior achilles tendon bursitis, fracture of the posterolateral talar tubercle, anterior achilles tendon bursitis, posterial tibial nerve neuralgia), disorders associated with metatarsalgia (such as interdigital nerve pain, metatarsophalangeal articulation pain, hallux rigidus), common hand disorders including deformities (such as Mallet finger, swan-neck deformity, Boutonniere deformity, erosive (inflammatory) osteoarthritis, Dupuytren's contracture), neurovascular syndromes (such as carpal tunnel syndrome, cubital tunnel syndrome, radial tunnel syndrome, Kienbock's disease, ganglia, reflex sympathetic dystrophy), trauma, infections, congenital deformities, tendon problem; and common sports injuries including metatarsal stress fracture, shin splints, popliteus tendinits, achilles tendinitis, patellofemoral pain, posterior femoral muscle strain, piriformis syndrome, lumbar strain, lateral epicondylitis, medial epicondylitis and rotator cuff tendinitis.

Other examples of therapeutic, cosmetic and research applications of the present invention include inflammatory diseases not related to skin or musculoskeletal and connective tissue such as multiple sclerosis, Crohn's disease and ulcerative colitis.

The present invention is further described by the following non-limiting examples.

### EXAMPLE 1

### Preparation of a Pharmaceutical Composition with an Water-in-Oil Microemulsion

Microemulsions were formed by mixing an oil phase composed of a mixture of oil and surfactants/co-surfactants with one ninth volume of water. The oil mixture consisted of caprylic/capric triglyceride (Crodamol GTCC; Croda) and medium chain mono- and di-glyceride (Capmul MCM; Croda) at a 3:1 ratio; and a surfactant/co-surfactant mixture at a 3:2 ratio; the oil to surfactant/co-surfactant ratio varying from 50:50 to 80:20. The surfactant was either polysorbate 40 (Crillet 2; Croda), polysorbate 60 (Crillet 3; Croda), or polysorbate 80 (Crillet 4; Croda). The co-surfactant was either sorbitan palmitate (Crill 2; Croda), sorbitan stearate (Crill 3; Croda) or. sorbitan monooleate (Crill 4; Croda). The final concentration of each pharmaceutical agent within the microemulsion was between 0.1 μg/ml to 100 mg/ml, each being dissolved in either the oil phase or water phase (as appropriate) prior to final mixing by gentle shaking.

### EXAMPLE 2

### Preparation of a Pharmaceutical Composition with an Water-in-Oil Microemulsion

Microemulsions were formed by mixing 3.5 ml of hexane (Sigma Chemical) with 1.5 g of surfactant and cosurfactant (Crillet 4 (Croda Surfactants) and Crill 4 (Croda Surfactants) at the ratio of 1:1) and adding 260 μl of protein solution at 10 mg/ml.

### EXAMPLE 3

### Preparation of a Pharmaceutical Composition with an Water-in-Oil Microemulsion

A stable microemulion was formed by mixing 16 g of oil (Crodamol GTCC (Croda Surfactants) and Capmul (Croda Surfactants), at 3:1 ratio) with 4 g of surfactant and cosurfactant (Brij 72 (Sigma Chemical) and Brij 97 (Sigma Chemical), at the ratio of 3:1) and stirring until clear. Water phase containing one or more water-soluble pharmaceutical agents was then added (0.5 ml; final concentration of pharmaceutical agent varied from 2 to 10 mg/ml). Microemulsion formation occurred following gentle shaking of the oil and water phases.

### EXAMPLE 4

### Transdermal Delivery of Various Proteins Including BSA

Microemulsions containing rhodamine-labeled-proteins were prepared in accordance with one of Examples 1 to 3. The microemulsions were then applied to the shaved skin of Balb/C mice. The microemulsion was left on the mice for 90 minutes, after which the mice were euthanased and the skin removed from mice and place in embedding media prior to frozen sectioning. Sections were stained with 50 μg/ml BisB to stain nuclei (blue).

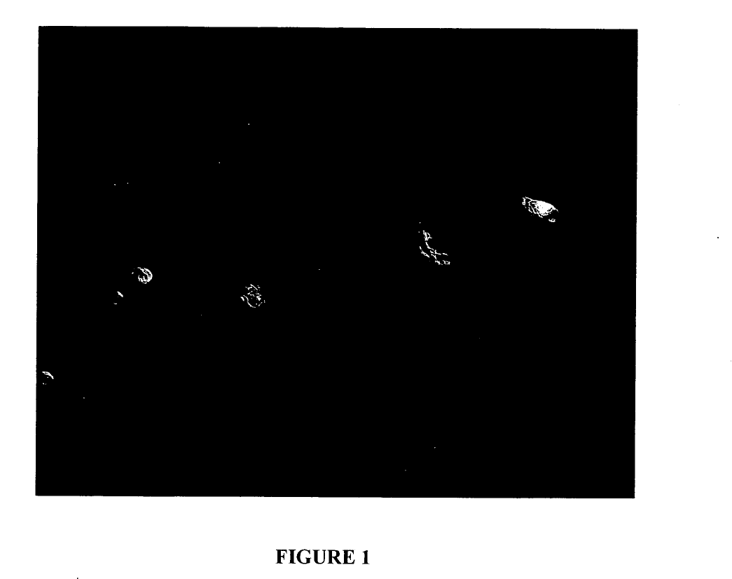

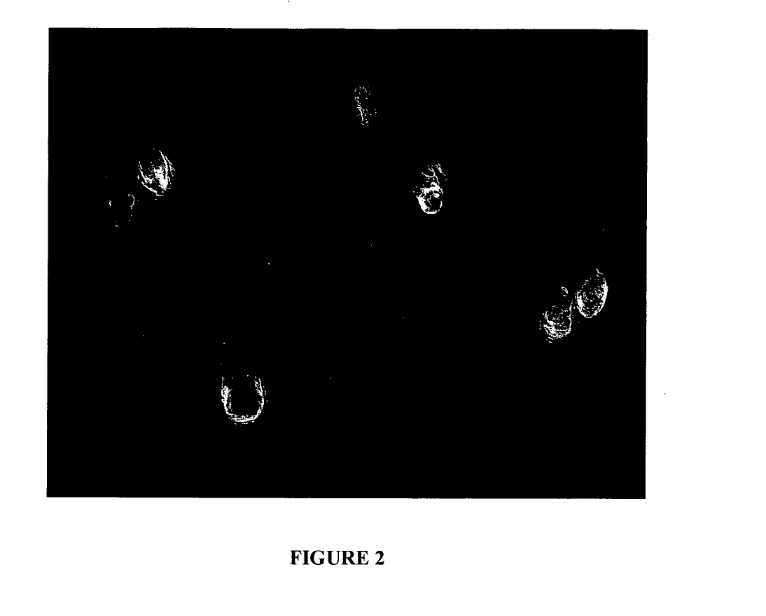

FIGS. 1 and 2 demonstrate the transdermal uptake of topically applied Rho-BSA. FIG. 1 is a photograph of rhodamine-labeled BSA found to have penetrated a hair follicle and extended to the bottom of the follicle. FIG. 2 is a photograph of rhodamine-labeled BSA found to have penetrated into the follicle-associated sebaceous gland.

### EXAMPLE 5

### Transdermal Delivery of ¹²⁵Labeled Proteins Applied in a Microemulsion

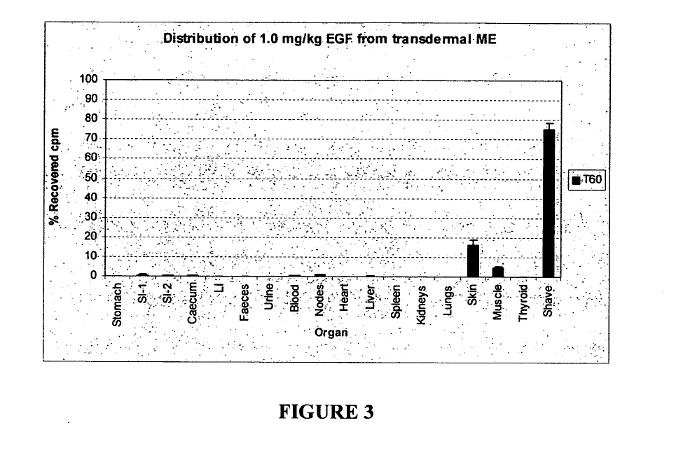

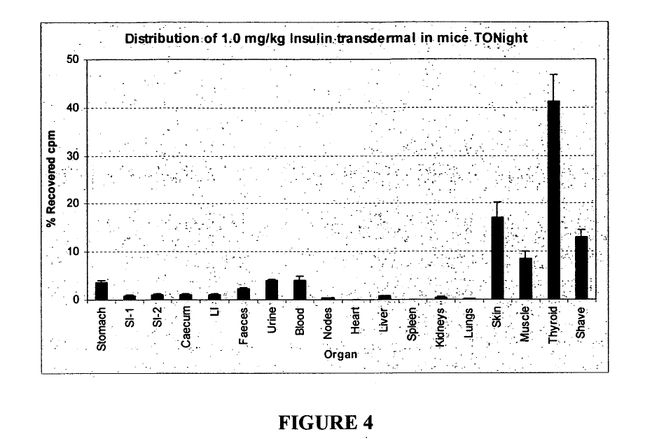

Microemulsions containing ¹²⁵I-labeled-proteins were prepared in accordance with one of Examples 1 to 3. The microemulsions were then applied to a 2×1 cm area of the shaved skin of Balb/C mice. The microemulsion was left on the mice and after 180 minutes or overnight the mice were euthanased and all the organs removed and counted in a gamma counter. FIGS. 3 and 4 shows the distribution of EGF and insulin in mice following transdermal application of EGF and insulin in a microemulsion formulation of the present invention after 3 hours and overnight, respectively.

As can be seen in FIGS. 3 and 4 there was the rapid appearance of ¹²⁵I in skin, muscle and the shaved area of the skin. Surprisingly there was also the appearance of counts in the stomach, SI-1, SI-2, caecum and faeces. Examination of the mice after microemulsion application showed that they frequently were seen to be grooming the site of application of the microemulsion. It was presumed that this was the reason for the appearance of counts in the organs of the intestine.

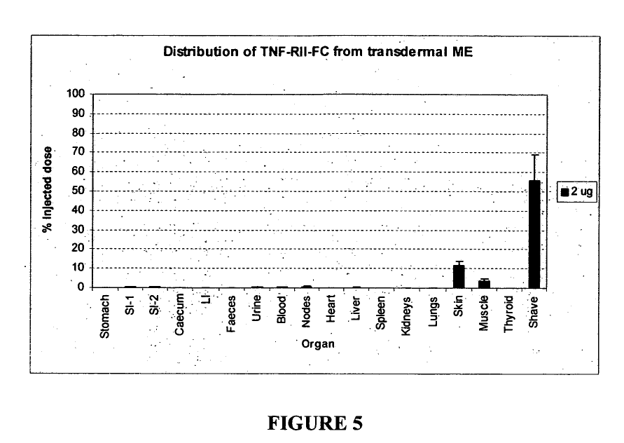

For this reason, a separate experiment was carried out as aforedescribed using microemulsions containing ¹²⁵I-labeled TNFR2-Fc, in which the mice were anaesthetized in order to stop the grooming behaviour. The TNFR2-Fc molecule was a recombinant human TNF receptor II-IgG1 Fc chimera, the production and purification of which is described in PCT Application No. WO 06/079176. The results from this experiment are shown in FIG. 5. It can be seen that when the mice were stopped from licking the site of application of the microemulsion, there was a greatly reduced amount of material found in the small intestinal tissues. There was, however, TGFR2-Fc found in the skin and the muscle. These data demonstrate that the microemulsion was capable of delivering the applied TGFR2-Fc to the muscle and adjacent skin tissue.

### EXAMPLE 6

### Treatment of a Dermalogical Disorder Using a Topical Pharmaceutical Composition of the Present Invention

The individual subjects of the in vivo studies described herein are warm-blooded vertebrate animals, which includes humans.

The clinical trial is subjected to rigorous controls to ensure that individuals are not unnecessarily put at risk and that they are fully informed about their role in the study. To account for the psychological effects of receiving treatments, volunteers are randomly assigned to topical placebo or topical treatment groups. Furthermore, to prevent the doctors from being biased in treatments, they are not informed as to whether the medication they are administering is the topical pharmaceutical composition or topical placebo. Using this randomization approach, each volunteer has the same chance of being given either the new treatment or the placebo.

Volunteers receive either a topical pharmaceutical composition, for example, one of those described in Examples 11, 12, 13 or 14 or topical placebo for an appropriate period, for example, a single 0.8 ml application on one psoriatic lesion (total skin area of 20 cm2) with a 7 day follow-up or, alternatively, multiple 0.8 ml applications on the same target lesion 9 times (every second day) over a 21 day period. Biological parameters associated with the indicated disease state or condition, for example, psoriasis, will be measured at the beginning (baseline measurements before any treatment), end (after the final treatment), and at regular intervals during the study period. Such measurements include the levels of TNF alpha in body fluids, tissues or organs compared to pre-treatment levels. Other measurements include, but are not limited to, indices of the disease state or condition being treated, body weight, blood pressure, serum titers of pharmacologic indicators of disease or toxicity as well as ADME (absorption, distribution, metabolism and excretion) measurements.

Information recorded for each patient includes age (years), gender, height (cm), family history of disease state or condition (yes/no), motivation rating (some/moderate/great) and number and type of previous treatment regimens for the indicated disease or condition.

Volunteers taking part in this study are adults aged 18 to 65 years and exhibit a particular dermalogical disorder, for example, psoriatic skin lesions. Additionally, roughly an equal number of males and females participate in the study. Volunteers with certain characteristics are equally distributed for topical placebo and topical pharmaceutical composition.

Evaluation of treatment is graded by an appropriate method, for example, the grading of treatment into one of four categories, namely, cured, obviously effective, effective and non-effective. “Cured” is where the inflammatory area on the plaque is diminished completely and the pruritus disappeared. “Obviously effective” is where the inflammatory area on the plaque is diminished by more than 60% and the pruritus is slighted and softened. “Effective” is where the inflammatory area on the plaque is diminished by 20 to 60% and the pruritus is slighted and softened. “Non-effective” is where the inflammatory area on the plaque is diminished by less than 20% or there is exacerbation of psoriasis.

Alternatively, treatment evaluation is graded by the Local Plaque Severity Index (LPSI), whereby each target plaque is assessed and rated for erthema, induration and dessquamation using a five-point scale by the supervising clinician at the time of the specified clinic visits. An example of an appropriate clinical visit timetable for the “multiple application” treatment regime is on days 0, 11 and 21. The five-point scale is defined as follows: 0=no symptoms; 1=slight; 2=moderate; 3=marked; 4=very marked. Scores for erthema, induration and desquamation are totalled. LPSI ranges from 0 to 12 with the highest score representing the more severe disease state.

In general, the volunteers treated with topical placebo have little or no response to treatment, whereas the volunteers treated with a topical pharmaceutical composition of the present invention show positive trends in their disease state or condition index at the conclusion of the study. In particular, the topical pharmaceutical composition of the present invention is obviously effective on most patients in the treatment group. No visible side-effects are observed.

### EXAMPLE 7

### Preparation of a Oil-Surfactant Phase Suitable for the Formation of a Microemulsion of the Present Invention

A 3:1 ratio of Crodamol GTCC to Capmul MCM C8 was prepared. A second mixture of a 3:2 ratio of Crillet 4 (Tween 80) to Crill 4 (Span 80) was also prepared. The oil-surfactant solution was prepared by mixing 1.4 g Crillet/Crill with 7.6 g Crodamol/Capmul. Alternative surfactants mixed at the similar ratios include Tween 60/Span 60 and Tween 40/Span 40, however, these material were heated in order to form the surfactant solution. The resulting oil-surfactant phase was then used to form microemulsions as described in Example 1.

### EXAMPLE 8

### Preparation of a Microemulsion Containing a Copper-Salicylic Acid Complex

Salicylic acid was dissolved at 43 mg/ml in distilled water. A molar equivalent of sodium hydroxide was added dropwise to the solution until the sodium-salicylate complex so formed became clear. One molar equivalent of dimethyl formamide was added to the sodium salicylate, after which 0.5 mole equivalent of copper chloride (100 mg/ml) was added dropwise to the solution. The solution gradually became turbid and a dark green colour became evident. After additional stirring a precipitate containing the Salicylate₂-DMF₂-Copper complex became evident. At this point the copper suspension was added to a 9-fold excess (volume:volume) of a microemulsion of the present invention. The solution became clear within minutes of gentle shaking as the microemulsion formed.

### EXAMPLE 9

### Preparation of a Microemulsion Containing a Copper-Ibuprofen Complex

Ibuprofen was dissolved at 53 mg/ml in distilled water. One molar equivalent of DMF (alternatives include, but are not limited to, DMSO, imidazoled, Pyridine) was added to the Ibuprofen, after which 0.5 mole equivalent of copper chloride (100 mg/ml) was added dropwise to the solution. The solution gradually became turbid and a dark blue colour became evident. After additional stirring a precipitate containing the Ibuprofen₂-DMF₂-Copper complex became evident. At this point the copper suspension was added to a 9-fold excess (volume:volume) of a microemulsion of the present invention. The solution became clear within minutes of gentle shaking as the microemulsion formed.

### EXAMPLE 10

### Preparation of a Microemulsion Containing a Copper-Penicillamine Complex

Preparation of the copper-penicillamine complex may also be formed by mixing two microemulsions containing separately the NSAID and copper. Thus, Penicillamine was dissolved at 75 mg/ml in DW. One molar equivalent of DMF (alternatives include, but are not limited to, DMSO, imidazole, Pyridine, Caffeine, Methylimidazole, 2-methylbenzimidazole, metronidazole, 2-methylimidazole, 3-picoline, 4-picoline, imidazole, 1-methylimidazole, diethylamine, nicotinamide, papaverine, N,N-dimethylacetamide, N-methyl-2-pyrrolidone, ethanol, methanol) was added to the Penicillamine, which was added to a 9-fold excess (volume:volume) of a microemulsion of the present invention. The solution became clear within minutes of gentle shaking as the microemulsion formed. Immediately 0.5 mole equivalent of copper chloride suspended in an microemulsion of the present invention (10 mg/ml) was added as a bolus to the solution. The solution gradually became turbid and a dark blue colour became evident. After additional stirring a precipitate containing the Ibuprofen₂-DMF₂-Copper complex became evident. At this point the copper suspension was added to a 9-fold excess (volume:volume) of a microemulsion of the present invention. The solution became clear within minutes of gentle shaking as the microemulsion formed.

### EXAMPLE 11

### Preparation of Pharmaceutical Compositions Containing VB12 Analogs in a Microemulsion

Vitamin B12 analogs, including cyanocobalamin, hydroxycobalamin, cobalamin, adenosyl-cobalamin (all obtained from Sigma Pharmaceuticals), were dissolved at 100 mg/ml in distilled water and used in the water phase to form separate microemulsions as described above in Examples 1-3, 7. The final concentration of each VB12 analog in the subsequent microemulsion was 10 mg/ml.

### EXAMPLE 12

### Preparation of Pharmaceutical Compositions Containing MLIF Molecules in a Microemulsion

Microemulsions containing MLIF (synthesized by Auspep, Melbourne, Australia), were formed as described in Examples 1-3 and 7. Specific MLIF peptide sequences were: MLIF A=H-Met-Gln-Glu-Asn-Ser-OH; MLIF B=H-Met-Gln-Ile-Asn-Ser-OH; MLIF C=H-Met-Gln-Met-Asn-Ser-OH. Final concentrations of the MLIF molecules and AdeCbl in the resulting microemulsions was 10 mg/ml.

### EXAMPLE 13

### Preparation of Pharmaceutical Compositions Containing Curucumin Analogs in a Microemulsion

Microemulsion containing tetrahydrocurcumin (10 mg/ml final concentration) was formed as described in Examples 1 or 7. Note that the tetrahydrocurcumin was added to the oil phase. Microemulsions containing a combination of tetrahydrocurcumin (2-50 mg/ml final concentration) and VB12 analogs (adenosyl-cobalamin at 10 mg/ml final concentration) were formed as described in Examples 1, 7 and 11.

### EXAMPLE 14

### Preparation of Pharmaceutical Compositions Containing Chimeric TNFR Molecules in a Microemulsion

Microemulsions containing a recombinant human TNF receptor II-IgG1 Fc chimera, the production and purification of which is described in PCT Application No. WO 06/079176, were formed as described in Examples 1, 2, 3, 7. The final concentration of the TNFRII-Fc chimeric protein in microemulsion was 5 mg/ml.

### EXAMPLE 15

### Topical Treatment of Carrageenan-Induced Inflammation in Mice Using the Pharmaceutical Composition of the Present Invention Containing VB12 Analogs

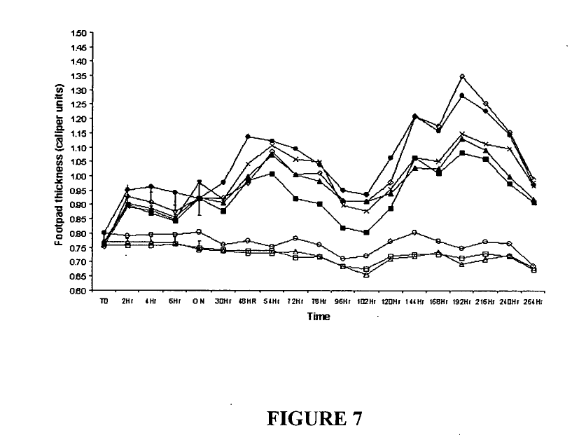

Inflammation was induced in the rear right footpad of C57/BI6 strain mice by an intra-pad injection of 50 μl of 1% Ilambda Carrageenan. Immediately following injection of Carrageenan into the foot-pad, mice receive treatment, which consisted of the application of 20 microL of the pharmaceutical compositions described above in Example 11, applied to a previously shaved area of the belly of the mouse using a flat applicator. The thickness of the Carageenan-injected foot and left rear foot (non-injected control foot) was measured with a constant pressure vernier calliper at regular timepoints for a total of 14 days. A comparison of foot thickness over time shows the effects of the pharmaceutical compositions containing various VB12 analogs (FIG. 7), all administered to a previously shaved area of the belly of each mouse using a flat applicator of a C57/BI6 strain mouse following Carrageenan-induced inflammation. VB12-CN=cyanocobalamin; OH-VB12=hydroxycobalamin; Cobal=cobalamin; Co-enzyme=co-enzyme VB12; All four pharmaceutical compositions were active in reducing inflammation within the right rear foot. The greatest reduction in inflammation occurred with the pharmaceutical composition containing co-enzyme VB12.

### EXAMPLE 16

### Topical Treatment of Carrageenan-Induced Inflammation in Mice Using the Pharmaceutical Compositions of the Present Invention Containing MLIF Molecules

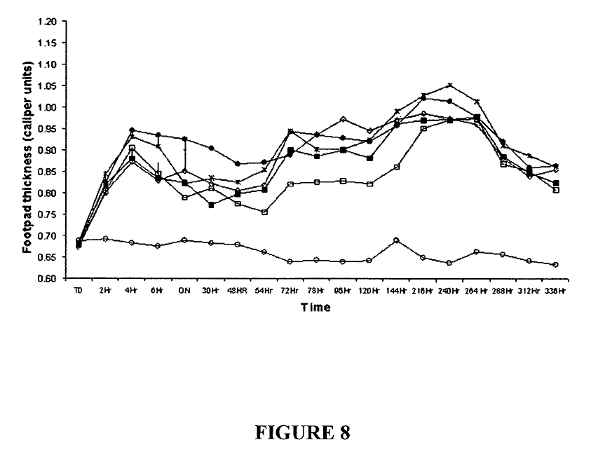

Inflammation was induced in the rear right footpad of C57/BI6 strain mice, as described above in Example 15. The pharmaceutical compositions described above in Example 12 were applied via the methods described above in Example 15 and foot thickness was measured as described above in Example 15. A comparison of foot thickness over time shows the effects of the pharmaceutical compositions containing various MLIF molecules (FIG. 8), all administered to a previously shaved area of the belly of each mouse using a flat applicator following Carrageenan-induced inflammation. OH=hydroxycobalamin. Application of all four pharmaceutical compositions resulted in a significantly lower level of inflammation in the rear right foot-pad than in untreated animals.

### EXAMPLE 17

### Topical Treatment of Carrageenan-Induced Inflammation in Mice Using the Pharmaceutical Compositions of the Present Invention Containing VB12 Analogs and/or Curucumin Analogs

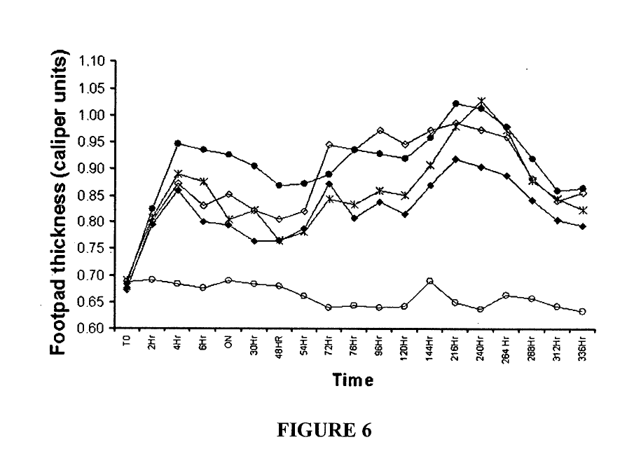

Inflammation was induced in the rear right footpad of C57/BI6 strain mice, as described above in Example 15. The pharmaceutical compositions described above in Example 13 were applied via the methods described above in Example 15 and foot thickness was measured as described above in Example 15. A comparison of foot thickness over time shows the effects of the pharmaceutical compositions containing adenosyl-cobalamin and/or tetrahydrocurcumin (FIG. 6), all administered to a previously shaved area of the belly of a C57/BI6 strain mouse following Carrageenan-induced inflammation. Ade or AdeCb=adenosyl-cobalamin; Tetra or TTC=tetrahydrocurcumin. Treatment with all three pharmaceutical compositions resulted in a significantly lower level of inflammation in the rear right foot-pad than in untreated animals. The greatest reduction in inflammation occurred with the pharmaceutical composition containing both adenosyl-cobalamin and tetrahydrocurcumin.

### EXAMPLE 18

### Topical Treatment of Carrageenan-Induced Inflammation in Mice Using the Pharmaceutical Compositions of the Present Invention Containing TNF Receptor-Fc Protein Analogs or anti-TNF Antibodies

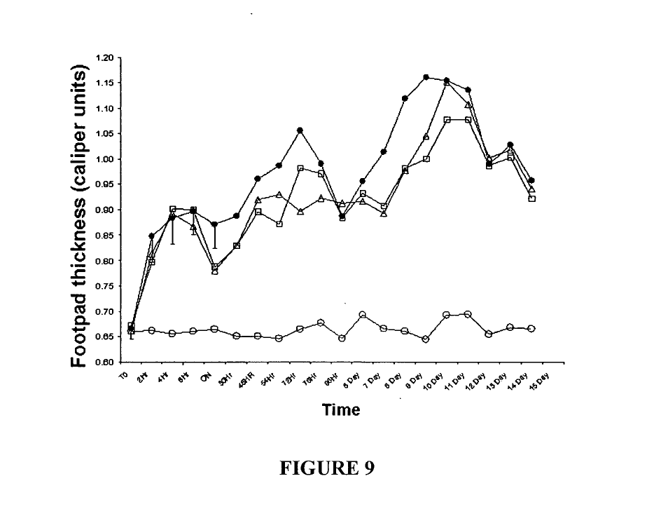

Inflammation was induced in the rear right footpad of C57/BI6 strain mice, as described above in Example 15. Pharmaceutical compositions were topically applied via the methods described above in Example 15 and foot thickness was measured as described above in Example 15. The pharmaceutical compositions were: a composition containing recombinant human TNF receptor II-IgG1 Fc chimera as described above in Example 14; a pharmaceutical composition containing adalimumab, a commercially available anti-TNF antibody (Humira®; Abbott; 4 mg/ml). Pharmaceutical compositions were applied via the methods described above in Example 15. Foot thickness was measured as described above in Example 15 and plotted over time (FIG. 9). Results show that treatment with either topical pharmaceutical composition resulted in significantly lower levels of inflammation in the rear right foot-pad than in untreated animals.

### EXAMPLE 19

### Topical Vaccination of Mice Using a Microemulsion of the Present Invention

Microemulsions for use as topical vaccines include a proteinaceous or non-proteinaceous antigen with or within an adjuvant. Ten mice per group were vaccinated with 1.8 LF (limit of flocculation units, 20 mcg) of tetanus toxoid (TT; CSL, Melbourne, Australia) either by injection of 0.05 ml saline solution into the quadriceps muscle with a 30 gauge needle or topical application of 0.04 ml of microemulsion containing TT, as described in Examples 1 and 7, to a 2 cm² patch of shaved skin on the belly. The control group received a mock vaccination of saline alone. Identical control and test groups were used in both the C57Black and BALB/C strains of mice. The C57Black strain is predisposed toward the development of Th1 or cell mediated immunity, associated with a protective response to bacteria and viruses. The BALB/C strain is predisposed toward development of Th2 or humoral immunity, associated with allergic reactions and immunity to gastrointestinal parasites. Mice were vaccinated every two weeks for a total of three vaccinations. Blood samples were taken by orbital bleeds 2 weeks post-vaccination. Indirect ELISA assay was used to determine the titre of serum IgG specific to tetanus toxoid using an anti-mouse whole IgG antibody alkaline phosphatase conjugate (Sigma Cat No. A3562). Cell mediated immunity was measured by the delayed type hypersensitivity reaction. The right footpad of vaccinated or control mice were injected with 10 mcg of tetanus toxoid in 0.05 ml of saline and the left footpad was injected with saline only as a control. The increase in footpad swelling of the right footpad was measured 24, 48 and 72 hours post-injection using a caliper.

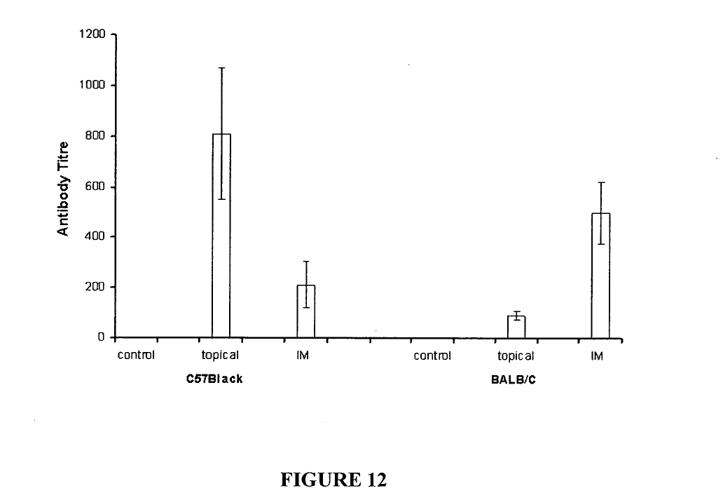

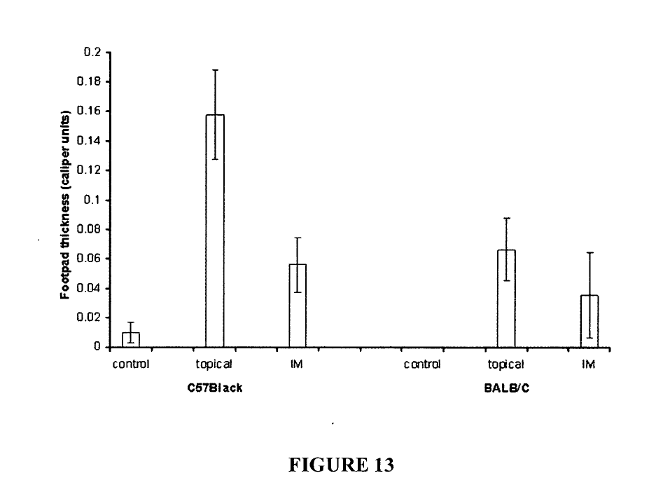

The antibody response to different vaccination regimes is plotted in FIG. 12. The increase in size of footpad is plotted in FIG. 13. It can be seen that topical administration of antigen in a microemulsion generated both an antibody and cellular response that was similar to intramuscular injection of the antigen.

### EXAMPLE 20

### Weight Loss Microemulsion

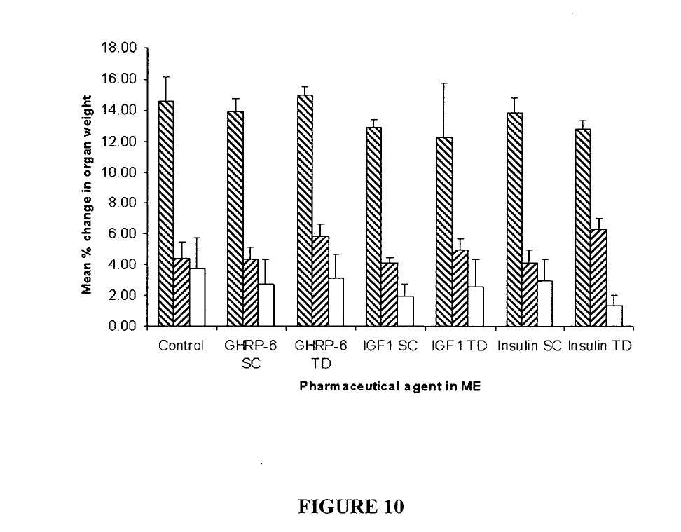

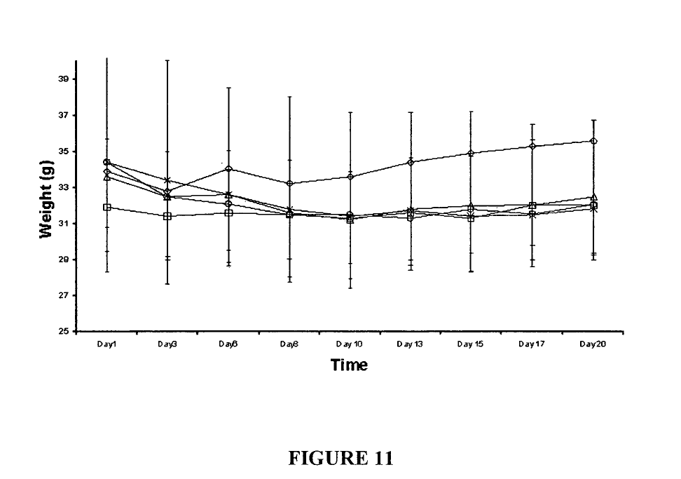

A microemulsion for topical weight loss was prepared according to the methods described in Examples 1 and 7, and included insulin, or insulin-like growth factor 1 (IGF1) or GHRP-6 at a final concentration of 1 mg/ml. A standardised volume of microemulsion was applied daily (TD) to an area of shaved skin on the belly of conscious Swiss strain mice. A standardised volume of each of insulin, or insulin-like growth factor 1 (IGF1) or GHRP-6 at a final concentration of 1 mg/ml in saline was injected sub cutaneously (SC) into the belly region of Swiss strain mice. Animal weights were measured 3 times per week. Following 2 weeks of treatment the mice were euthanased and their tissues removed and weighed. The weight of the animals with time is plotted in FIG. 11. It can be seen that there was a decrease in body weight of animals treated with the topical insulin of up to 15% within 2 weeks (FIG. 11). Examination of organ weights showed the greatest change in the topically applied insulin group, where the weight of skin was significantly reduced whilst muscle mass increased (FIG. 10).

### EXAMPLE 21

### Treatment of Carcinoma Using a Topical Pharmaceutical Composition of the Present Invention

The individual subjects of the in vivo studies described herein are warm-blooded vertebrate animals, which includes humans.

Preferably to account for the psychological effects of receiving treatments, the trial is conducted in a double-blinded fashion. Volunteer patients at a tertiary care center are randomly assigned to placebo or treatment groups. Furthermore, to prevent the doctors from being biased in treatments, they are not informed as to whether the medication they are administering is the topical pharmaceutical composition of the present invention or a placebo composition. Using this randomization approach, each volunteer has the same chance of being given either the new treatment or the placebo treatment.

Volunteers presenting with, for example, cutaneous melanoma, receive either the topical pharmaceutical composition of the present invention, for example, a microemulsion as described in Examples 1, 2, 3 or 7 with one or more of the following: 5-fluorouracil, carboplatin, oxaliplatin, cis-platin, selenomethionine, chlorotoxin, L49 monoclonal antibody, MAb-LM609, antileukinate, gold-monophosphine and gold-diphosphine, pyridoxal isonicotinoyl hydrazone (PIH), amino-substituted platinum complexes, paclitaxol, defoxamine, synthetic retinoids such as tretinoin, isotretinoin, and etretinate, imiquimod (e.g. 5%), calcitriol, temozolomide, interferon beta, betulinic acid (e.g. 20%), a curcuminoid derivative, TSA, or topical placebo composition, for an appropriate period thereafter with biological parameters associated with the condition being measured at the beginning (baseline measurements before any treatment), end (after the final treatment), and at regular intervals during the study period. Such measurements include the levels of each pharmaceutical agent in the composition in body fluids, tissues or organs compared to pre-treatment levels. Other measurements include, but are not limited to, evidence of metastisis in tissues such as lymph nodes. Still other measurements include, but are not limited to body weight, blood pressure, serum titers of pharmacologic indicators of disease such as specific disease indicators or toxicity as well as ADME (absorption, distribution, metabolism and excretion) measurements.

Information recorded for each patient includes age (years), gender, height (cm), family history of disease state or condition (yes/no), motivation rating (some/moderate/great) and number and type of previous cancer treatment regimens.

Volunteers taking part in this study are adults aged 18 to 65 years. Volunteers with certain characteristics are equally distributed for topical placebo treatment and the topical pharmaceutical composition of the present invention treatment. In general, at the conclusion of the study the volunteers topically treated with the placebo composition have little or no response to treatment, whereas the volunteers treated with the topical pharmaceutical composition of the present invention show positive trends in one or more of the following parameters: overall survival (OS), disease-free survival (DFS).

### EXAMPLE 22

### Gene Therapy Treatment of Cancer Using a Topical Pharmaceutical Composition of the Present Invention Containing Nucleic Acid Molecules

Recent methods indicate that cancerous disease states associated with the expression of abnormal common fusion genes can be treated by the specific silencing of those genes via the administration of siRNA (Hashimoto et al. PNAS 101(17): 6647-6652, 2004).

The individual subjects of the in vivo studies described herein are warm-blooded vertebrate animals, which includes humans.

Preferably to account for the psychological effects of receiving treatments, the trial is conducted in a double-blinded fashion. Volunteers presenting with a cancerous condition, for example, chronic myeloid leukemia, are randomly assigned to placebo or treatment groups. Furthermore, to prevent the doctors from being biased in treatments, they are not informed as to whether the medication they are administering is the topical pharmaceutical composition of the present invention or a topical placebo. Using this randomization approach, each volunteer has the same chance of being given either the new treatment or the placebo.

Volunteers receive either the topical pharmaceutical composition of the present invention, for example, a microemulsion as described in Examples 1, 2, 3 or 7 containing siRNAs directed against one or more of the two types of the common fusion genes present in chronic myeloid leukemia patients (Hashimoto et al., supra) or placebo for an appropriate period with biological parameters associated with the indicated disease state being measured at the beginning (baseline measurements before any treatment), end (after the final treatment), and at regular intervals during the study period. Such measurements include the levels of pharmaceutical agent in the composition in body fluids, tissues or organs compared to pre-treatment levels. Other measurements include, but are not limited to, counts of abnormal white cells, other specific tests for detecting trace leukemia cells, body weight, blood pressure, serum titers of pharmacologic indicators of disease such as specific disease indicators or toxicity as well as ADME (absorption, distribution, metabolism and excretion) measurements.

Information recorded for each patient includes age (years), gender, height (cm), family history of disease state or condition (yes/no), motivation rating (some/moderate/great) and number and type of previous treatment regimens for the indicated disease or condition.

Volunteers taking part in this study are adults aged 18 to 65 years and roughly an equal number of males and females participate in the study. Volunteers with certain characteristics are equally distributed for placebo and the topical pharmaceutical composition of the present invention treatment. In general, the volunteers treated with placebo have little or no response to treatment, whereas the volunteers treated with the topical pharmaceutical composition of the present invention show positive trends in one or more of the following parameters: decreased counts of abnormal white cells, overall survival (OS), disease-free survival (DFS) at the conclusion of the study.

### EXAMPLE 23

### Treatment of Neuropathic or Nociceptic Pain Using a Topical Pharmaceutical Composition of the Present Invention Containing Analgesic Agents

The individual subjects of the in vivo studies described herein are warm-blooded vertebrate animals, which includes humans.

Preferably to account for the psychological effects of receiving treatments, the trial is conducted in a double-blinded fashion. Volunteers presenting with either neuropathic or nociceptic pain are randomly assigned to placebo or treatment groups. Furthermore, to prevent the doctors from being biased in treatments, they are not informed as to whether the medication they are administering is the topical pharmaceutical composition of the present invention or a topical placebo. Using this randomization approach, each volunteer has the same chance of being given either the new treatment or the placebo.

Volunteers receive either the topical pharmaceutical composition of the present invention, for example, a microemulsion as described in Examples 8-14, optionally containing one or more analgesic agents such as lidocaine (4%) or placebo for an appropriate period with biological parameters associated with the indicated disease state or condition being measured at the beginning (baseline measurements before any treatment), end (after the final treatment), and at regular intervals during the study period. Such measurements include the levels of each pharmaceutical agent in the composition in body fluids, tissues or organs compared to pre-treatment levels. Other measurements include, but are not limited to, indices of the disease state or condition being treated, body weight, blood pressure, serum titers of pharmacologic indicators of disease such as specific disease indicators or toxicity as well as ADME (absorption, distribution, metabolism and excretion) measurements.

Information recorded for each patient includes age (years), gender, height (cm), family history of disease state or condition (yes/no), motivation rating (some/moderate/great) and number and type of previous treatment regimens for the indicated disease or condition.

Volunteers taking part in this study are adults aged 18 to 65 years and roughly an equal number of males and females participate in the study. Volunteers with certain characteristics are equally distributed for placebo and the topical pharmaceutical composition of the present invention treatment. In general, the volunteers treated with placebo have little or no response to treatment, whereas the volunteers treated with the topical pharmaceutical composition of the present invention show an amelioration of pain intensity at the conclusion of the study.

### EXAMPLE 24

### Treatment of Bruising with a Microemision Containing Adenosyl Cobalamin and Tetrahydrocurcumin

A microemulsion containing adenosyl cobalamin and tetrahydrocurcumin was prepared according to Example 13. A human subject, who had encountered a sharp blow to the shins with a cricket ball, was treated as soon as practical with the microemulsion, and once again after several hours. Twentyfour hours later the subject was visually examined and found to have no signs of bruising at the site of injury.

### EXAMPLE 25

### Treatment of Rheumatoid Arthritis with a Microemision Containing Adenosyl Cobalamin and Tetrahydrocurcumin

A microemulsion containing adenosyl cobalamin and tetrahydrocurcumin was prepared according to Example 13. A human subject, who had chronic arthritis of the hand was treated with the microemulsion daily. The subject reported increased mobility of the joints with a concurrent reduction in pain.

### EXAMPLE 26

### Treatment of a Sprained Ankle with a Microemision Containing Adenosyl Cobalamin and Tetrahydrocurcumin

A microemulsion containing adenosyl cobalamin and tetrahydrocurcumin was prepared according to Example 13. A human subject, who had recently badly sprained her ankle was treated with the microemulsion daily, with an accompanying compression bandage. The subject reported both a reduction in swelling and pain than generally experienced with a similar injury.

### EXAMPLE 27

### Treatment of Contraction-Induced Injury in Mice Using the Pharmaceutical Composition of the Present Invention

Contraction-induced injury is induced in a suitable strain of mice, for example, C57/BI6 strain mice by a suitable method, for example, the lengthening contraction protocol (LCP) of Rader and Faulkner J Appl Physiol 101:887-892, 2006.

Immediately following induction of the injury, the mice receive treatment, consisting of the application to the skin area adjacent to the plantar flexor muscle group using a flat applicator of either a suitable volume of the pharmaceutical composition, for example, that described in Example 13, or a topical placebo composition. The treatment is administered at regular time-points for a total of 1-2 months.

At regular time-points following the LCP injury, for example, at 1, 3, 5 days, 1 week, and 2 weeks, various physical and biological parameters are measured. Such measurements include the maximum isometric force generated and the weight of the isolated plantar flexor muscle group (see Rader and Faulkner, supra).

In general, at the conclusion of the study, mice treated with the topical pharmaceutical composition of the present invention show positive trends in maximum isometric force generation and muscle weight when compared with mice treated with the topical placebo composition.

### EXAMPLE 28

### Treatment of Delayed Onset Muscle Soreness (DOMS) Using a Topical Pharmaceutical Composition of the Present Invention

The individual subjects of the in vivo studies described herein are warm-blooded vertebrate animals, which includes humans.

Preferably to account for the psychological effects of receiving treatments, the trial is conducted in a double-blinded fashion. Volunteers are randomly assigned to placebo or treatment groups. Furthermore, to prevent the participating clinicians from being biased in treatments, they are not informed as to whether the treatment being administering is the topical pharmaceutical composition of the present invention or a topical placebo composition. Using this randomization approach, each volunteer has the same chance of being given either the new treatment or the placebo.

Volunteers take part in a suitable exercise protocol to induce lower limb fatigue, for example, repetitive sets of jumping movements on an inclined sled apparatus on three separate days with a minimum of one week rest between each test session (presentation by S. Hughes at Australian Conference of Science and Medicine in Sport, Yanuca, Fiji, October, 2006).

Volunteers receive a controlled application of either the topical pharmaceutical composition of the present invention, for example, that described in or topical placebo composition applied to the lower limbs at regular time-points after the initial exercise protocol for a total of 1-14 days.

Physical and biological parameters associated with DOMS are measured at the beginning (baseline measurements before any treatment), end (after the final treatment), and at regular intervals during the study period. Suitable physical parameters include the level of performance between initial and follow-up test sessions of the exercise protocol. Suitable biological parameters include the levels of pharmaceutical agent or agents of the composition in body fluids, tissues or organs compared to pre-treatment levels. Other measurements include, but are not limited to, blood lactate levels, body weight, blood pressure, serum titers of pharmacologic indicators of DOMS such as creatinine levels or toxicity as well as ADME (absorption, distribution, metabolism and excretion) measurements.

Information recorded for each patient includes age (years), gender, height (cm), family history of disease state or condition (yes/no), motivation rating (some/moderate/great) and number and type of previous treatment regimens for DOMS.

Volunteers taking part in this study are adults aged 18 to 65 years and roughly an equal number of males and females participate in the study. Volunteers with certain characteristics are equally distributed for topical placebo composition and topical pharmaceutical composition of the present invention treatments. In general, at the conclusion of the study, volunteers treated with the topical pharmaceutical composition of the present invention show positive trends in one or more of the above-mentioned physical or biological parameters in comparison to volunteers treated with the topical placebo composition.

### EXAMPLE 29

### Treatment of Facial Bruising Associated with Facial Surgery Using a Topical Pharmaceutical Composition of the Present Invention

The individual subjects of the in vivo studies described herein are warm-blooded vertebrate animals, which includes humans.

Preferably to account for the psychological effects of receiving treatments, the trial is conducted in a double-blinded fashion. Volunteer patients undergoing rhytidectomy at a tertiary care center are randomly assigned to placebo or treatment groups. Furthermore, to prevent the doctors from being biased in treatments, they are not informed as to whether the medication they are administering is the topical pharmaceutical composition of the present invention or a placebo composition. Using this randomization approach, each volunteer has the same chance of being given either the new treatment or the placebo treatment.

Volunteers receive either the topical pharmaceutical composition of the present invention, for example, that described in Example 13 or topical placebo composition peri-operatively and for an appropriate period thereafter with physical and biological parameters associated with post-operative facial surgery being measured at the beginning (baseline measurements before any treatment), end (after the final treatment), and at regular intervals during the study period. Such measurements include the levels of pharmaceutical agent in the composition in body fluids, tissues or organs compared to pre-treatment levels. Other measurements include, but are not limited to, standardized objective methods for measuring physical parameters of skin, for example, skin color changes and total area of ecchymosis, as well as subjective assessments of postoperative ecchymosis (Seeley et al. Arch Facial Plast Surg 8:54-59, 2006). Still other measurements include, but are not limited to body weight, blood pressure, serum titers of pharmacologic indicators of disease such as specific disease indicators or toxicity as well as ADME (absorption, distribution, metabolism and excretion) measurements.

Postoperative photographs are analyzed using a suitable computer model for color changes and area of ecchymosis, for example, the analysis method described by Seeley et al. supra. Subjective assessments of post-operative ecchymosis were obtained from the relevant clinicans and from volunteers (self-assessments).

Information recorded for each patient includes age (years), gender, height (cm), family history of disease state or condition (yes/no), motivation rating (some/moderate/great) and number and type of previous facial treatment regimens.

Volunteers taking part in this study are adults aged 18 to 65 years. Volunteers with certain characteristics are equally distributed for topical placebo treatment and the topical pharmaceutical composition of the present invention treatment. In general, at the conclusion of the study the volunteers topically treated with the placebo composition have little or no response to treatment, whereas the volunteers treated with the topical pharmaceutical composition of the present invention show positive trends in one or more of the following parameters: skin color changes; total area of ecchymosis; clinician-assessed post-operative ecchymosis; degree of self-perceived ecchymosis.

### EXAMPLE 30

### Detection of Sentinel Lymph Nodes in Patients with an Identified Primary Tumour Using a Topical Pharmaceutical Composition of the Present Invention

The individual subjects of the in vivo studies described herein are warm-blooded vertebrate animals, which includes humans.

Preferably to account for the psychological effects of receiving treatments, the trial is conducted in a double-blinded fashion. Volunteer patients at a tertiary care center are randomly assigned to placebo or treatment groups. Furthermore, to prevent the doctors from being biased in treatments, they are not informed as to whether the medication they are administering is the topical pharmaceutical composition of the present invention or a placebo composition. Using this randomization approach, each volunteer has the same chance of being given either the new treatment or the placebo treatment.

Volunteers presenting with a tumour, for example, a cutaneous melanoma, receive either the topical pharmaceutical composition of the present invention, for example, a microemulsion as described in Examples 1, 2, 3 or 7 with ^{99m}Tc chelated to DTPA, or topical placebo composition, applied to the tumour. At an appropriate time after the application of the composition, the body region is imaged for the detection of imaging agent. It is anticipated that the accumulation of imaging agent within the sentinel lymph node will allow for a rapid identification and subsequent removal of the lymph node. Other measurements include, but are not limited to body weight, blood pressure, serum titers of pharmacologic indicators of disease such as specific disease indicators or toxicity as well as ADME (absorption, distribution, metabolism and excretion) measurements.

Information recorded for each patient includes age (years), gender, height (cm), family history of disease state or condition (yes/no), motivation rating (some/moderate/great) and number and type of previous cancer treatment regimens.

Volunteers taking part in this study are adults aged 18 to 65 years. Volunteers with certain characteristics are equally distributed for topical placebo treatment and the topical pharmaceutical composition of the present invention treatment. In general, at the conclusion of the study the volunteers treated with the topical pharmaceutical composition of the present invention, when compared with the volunteers topically treated with the placebo composition, show positive trends in one or more of the following parameters: incidence of metastisis, overall survival (OS), disease-free survival (DFS).

### EXAMPLE 31

### Sub Cutaneous Fat Loss in Volunteers Following Topical Application of the Pharmaceutical Composition of the Present Invention Containing Insulin

The individual subjects of the in vivo studies described herein are warm-blooded vertebrate animals, which includes humans.

Preferably to account for the psychological effects of receiving treatments, the trial is conducted in a double-blinded fashion. Volunteers are randomly assigned to placebo or treatment groups. Furthermore, to prevent the doctors from being biased in treatments, they are not informed as to whether the medication they are administering is the topical pharmaceutical composition of the present invention, or a placebo. Using this randomization approach, each volunteer has the same chance of being given either the new treatment or the placebo.

Volunteers receive either the topical pharmaceutical composition of the present invention, for example, a microemulsion as described in Examples 1, 2, 3 or 7 with one or more of the following: insulin (final concentration of 0.5-2 mg/ml), insulin-like growth factor 1 (IGF1) or GHRP-6 or placebo onto a suitable part of the body, for example, the anterior and posterior aspects of the upper arm for an appropriate period with particular physical and biological parameters associated with the condition being measured at the beginning (baseline measurements before any treatment), end (after the final treatment), and at regular intervals during the study period. Suitable physical measurements include skinfold caliper measurements or A- and/or B-mode ultrasound measurements of the thickness of the subcutaneous fat layer overlying the upper arm. Suitable biological measurements include blood levels of insulin and glucose in body fluids, tissues or organs compared to pre-treatment levels. Other measurements include, but are not limited to, body weight and blood pressure as well as ADME (absorption, distribution, metabolism and excretion) measurements.

Information recorded for each patient includes age (years), gender, height (cm), family history of condition (yes/no), motivation rating (some/moderate/great) and number and type of previous treatment regimens for the indicated condition.

Volunteers taking part in this study are adults aged 18 to 65 years and roughly an equal number of males and females participate in the study. Volunteers with certain characteristics are equally distributed for placebo and the topical pharmaceutical composition of the present invention treatment. At the conclusion of the study, the volunteers treated with the topical pharmaceutical composition of the present invention show reduced thickness of sub cutaneous fat deposits of the upper arm, as determined by one or more of the following methods: skinfold caliper measuremen, A-mode ultrasound measurement, B-mode ultrasound measurement; whereas the volunteers treated with placebo have little or no response to treatment. It is anticipated that blood levels of insulin, insulin-like growth factor 1 (IGF1) or GHRP-6 are not affected by treatment with the topical pharmaceutical composition of the present invention.

### EXAMPLE 32

### Treatment of Erectile Dysfunction Using a Topical Pharmaceutical Composition of the Present Invention

The individual subjects of the in vivo studies described herein are warm-blooded vertebrate animals, which includes humans.

Preferably to account for the psychological effects of receiving treatments, the trial is conducted in a double-blinded fashion. Volunteers are randomly assigned to placebo or treatment groups. Furthermore, to prevent the doctors from being biased in treatments, they are not informed as to whether the medication they are administering is the topical pharmaceutical composition of the present invention or a placebo. Using this randomization approach, each volunteer has the same chance of being given either the new treatment or the placebo.

Volunteers presenting with erectile dysfunction receive either a topical pharmaceutical composition of the present invention, for example, a microemulsion as described in Examples 1, 2, 3 or 7 with a PDE5 inhibitor (e.g. sildenafil, tadalafil, vardenafil) or placebo for an appropriate period with physical and biological parameters associated with the indicated disease state or condition being measured at the beginning (baseline measurements before any treatment), end (after the final treatment), and at regular intervals during the study period. Such measurements include the levels of PDE5 inhibitors in body fluids, tissues or organs compared to pre-treatment levels. Other measurements include, but are not limited to, indices of erectile dysfunction, body weight, blood pressure and toxicity as well as ADME (absorption, distribution, metabolism and excretion) measurements.

Information recorded for each patient includes age (years), height (cm), family history of disease state or condition (yes/no), motivation rating (some/moderate/great) and number and type of previous treatment regimens for erectile dysfunction.

Volunteers taking part in this study are adult males aged 18 to 65 years. Volunteers with certain characteristics are equally distributed for placebo and the topical pharmaceutical composition of the present invention treatment. In general, the volunteers treated with placebo have little or no response to treatment, whereas the volunteers treated with the topical pharmaceutical composition of the present invention show positive trends in their disease state or condition index at the conclusion of the study.

### EXAMPLE 33

### Treatment of Intradermal Melanoma in Mice Using a Topical Pharmaceutical Composition of the Present Invention

Mice (Balb/C and C57/BI) were injected intradermally or sub-cutaneously with 1×10⁶ cells of the B16 melanoma line. The tumour was allowed to grow until it was approximately 0.5 gm in mass. The mice were then treated topically with the following chemotherapeutic agents dissolved in a microemulsion of prepared according to Examples 1-3, 7: 5-fluorouracil (10 mg/ml, 20 microlitre dose, 2 times per day); Carboplatin (10 mg/ml, 20 microlitre dose, 2 times per day); Cis-Platinum (10 mg/ml, 20 microlitre dose, 2 times per day); or a control microemulsion without chemotherapeutic agents. After 4 days of treatment, there was no reduction in body weight of the animals, although in each group there was a reduction in growth rate of the tumour.

Those skilled in the art will appreciate that the invention described herein is susceptible to variations and modifications other than those specifically described. It is to be understood that the invention includes all such variations and modifications. The invention also includes all of the steps, features, compositions and compounds referred to, or indicated in this specification, individually or collectively, and any and all combinations of any two or more of said steps or features.

### BIBLIOGRAPHY

Atherton and Shephard Chapter 9 entitled “Peptide Synthesis” by which is included in a publication entitled “Synthetic Vaccines”
Escribano et al. European Journal of Pharmaceutical Sciences 19:203-210, 2003
Hashimoto et al. PNAS 101(17): 6647-6652, 2004
Kreilgaard et al. Journal of Controlled Release 69:421-433, 2000
Li et al. International Journal of Pharmaceutics 237:77-85, 2002
Rader and Faulkner J Appl Physiol 101:887-892, 2006
S. Hughes at Australian Conference of Science and Medicine in Sport, Yanuca, Fiji, October, 2006
Santus et al. J Control Release 25:1-20,1993
Seeley et al. Arch Facial Plast Surg 8:54-59, 2006
Shahrokh et al. Therapeutic Protein and Peptide Formulation and Delivery, American Chemical Society, 1997
Sintov et al. Journal of Controlled Release 95:173-183, 2004
Valenta et al. Journal of Controlled Release 95:257-265, 2004

1. A composition comprising a water-in-oil microemulsion and one or more pharmaceutically or physiologically active agents wherein said composition delivers the agent(s) to a subject transdermally.

2. The composition of claim 1, wherein the agent is selected from the list consisting of a protein, chemical compound, steroid, hormone, nucleic acid molecule and a vitamin.

3. The composition of claim 2, wherein the protein is selected from the list consisting of a protein in the TNF superfamily, a chemokine, an interleukin, an interleukin receptor, an interleukin receptor antagonist, an interferon, a lectin, a NSAID, a growth factor, a growth factor receptor, an embryonic growth factor, an adhesion molecule, a cytokine, a hormone, an antibiotic, an antibody, an enzyme and an antigen.

4. The composition of claim 3, wherein the protein comprises a protein-Fc.

5. The composition of claim 4, wherein the protein-Fc is selected from the list consisting of TNFR1-Fc, TNFR2-Fc, OX-40-Fc, MC148-Fc, IL-1Ra-Fc, IL-2Ra-Fc, IL-2Rb-Fc, IL-2Rg-Fc, IL-3Ra-Fc, IL-4Ra-Fc, IL-5Ra-Fc, IL-6Ra-Fc, IL-7Ra-Fc, IL-10Ra-Fc, IL-11Ra-Fc, IL-13Ra-Fc, IL-15Ra-Fc, IFN-aRa-Fc, IFN-aRb-Fc, IFN-gRa-Fc, IFN-gRb-Fc, CD209L-Fc, E-Selectin-Fc, L-Selectin-Fc, P-Selectin-Fc, EGFR-Fc, FGFR1-Fc, FGFR4-Fc, FGFR5-Fc, Flt3-Fc, hGHR-Fc, NGFR-Fc, TGFbR2-Fc, Trk-A-Fc, Trk-B-Fc and VEGFR-Fc.

6. The composition of claim 2, wherein the agent is a NSAID.

7. The composition of claim 2, wherein the agent is a copper complex of a NSAID.

8. The composition of claim 6, wherein the NSAID is selected from the list consisting of aspirin, choline magnesium trisalicylate, dilfunisal, salasalate, benorylate, carpofen, fenoprofen, fluribiprofen, ibuprofen, ketoprofen, naproxen, naproxen Na, suprofen, oxaprozin, alclofenac, diclofenac, fenclofenac, indomthacin, sulindac, tolemetin, isoxicam, meloxicam, piroxicam, tenoxicam, flufenamic acid, meclofenamate, mefenamic acid, nabumetone, niflumic acid, etodolan, phenylbutazone, oxyphenobutazone, ketorolac, celecoxib, parecoxib, valdecoxib and rofecoxib.

9. The composition of claim 2, wherein the vitamin is Vitamin B12, Vitamin E or Vitamin C or derivatives or analogs thereof.

10. The composition of claim 1, wherein the agent is selected from the list consisting of a nitric oxide chelator, a nitric oxide synthase inhibitior, a curcuminoid, a COX1 inhibitor and a COX2 inhibitor.

11. The composition of claim 1, wherein the agent is an antibody specific for IL-20, C5aR, IFN alpha, TNF, IL-12, IL-1, IL-6, a leukotriene or IL-4.

12. The composition of claim 1, wherein the agents are adenosylcobalamin and a curcuminoid.

13. The composition of claim 12 wherein the curcuminoid is tetrahydrocurcumin.

14. The composition of claim 1 wherein the agents are a vaccine antigen and an adjuvant.

15. The composition of claim 14 wherein the adjuvant is selected from the list consisting of aluminium hydroxide, aluminium phosphate and calcium phosphate. In addition, oil based emulsions and products from bacteria including synthetic derivatives and liposomes of gram-negative bacteria, endotoxins, cholesterol, fatty acids, aliphatic amines, parafinic and vegetable oils, monophosphoryl lipid A, iscoms with Quil-A and Syntex agents formulations containing threonyl derivaties or muramyl type peptides may be employed. Other adjuvants contemplated by the present invention include Freund's emulsify oil adjuvants (complete and incomplete), ARLACEL A, mineral oil, emulsified peanut oil adjuvant (adjuvant 65), mineral compounds and bacterial products from Bordetella pertussis, Corynebacterium granulosom-derived P40 component, lipopolysaccaride, Mycobacterium or components thereof, inulin, cholera toxin and a liposome.

16. The composition of claim 2 wherein the agent is insulin.

17. The composition of claim 1 wherein the agent is an imaging agent selected from the list consisting of Antimony-124, Antimony-125, Arsenic-74, Barium-103, Barium-140, Beryllium-7, Bismuth-206, Bismuth-207, Cadmium-109, Cadmium-11Sm, Calcium-45, Cerium-139, Cerium-141, Cerium-144, Cesium-137, Chromium-51, Cobalt-56, Cobalt-57, Cobalt-58, Cobalt-60, Cobalt-64, Erbium-169, Europium-152, Gadolinium-153, Gold-195, Gold-199, Hafnium-175, Hafnium0175-181, Indium-111, Iridium-192, Iron-55, Iron-59, Krypton-85, Lead-210, Manganese-54, Mercury-197, Mercury-203, Molybdenum-99, Neodymium-147, Neptunium-237, Nickel-63, Niobium-95, Osmium-185+191, Palladium-103, Platinum-195m, Preseodymium-143, Promethium-147, Protactinium-233, Radium0226, Rhenium-186, Rubidium-86, Ruthenium-103, Ruthenium-106, Scandium-44, Scandium-46, Selenium-75, Silver-110m, Silver-111, Sodium-22, Strontium-85, Strontium-89, Strontium-90, Sulfur-35, Tantalum-82, Technetium-99m, Tellurium-125, Tellurium-132, Thallium-204, Thorium-228, Throium-232, Thallium-170, Tin-113, Titanium-44, Tungsten-185, Vanadium-48, Vanadium-49, Ytterbium-169, Yttrium-88, Yttrium-90, Yttrium-91, Zinc-65 and Zirconium-95.

18. The composition of claim 1 wherein the agent is a chelating agent.

19. The composition of claim 18 wherein the chelating agent is selected from the list consisting of 1,3,5triaminocyclohexane; 1,3,5triaminocyclohexane N-pyridine; 1,1-cyclobutanedicarboxylic acid; 1,2-Dimethyl-3-hydroxypyridin-4-one; 1,2-dimethyl-3-hydroxypyridin-4-one (Deferiprone); 1,4,7,10-tetraaza-4,7,10-tris(carboxymethyl)-1-cyclododecylacetyl-R-(+)-R-methylbenzylamine (DOTA-MBA); 1,4,7,10-tetraazacyclododecane-N,N,N,N-tetraacetic acid (DOTA); 1,6-dimethyl-2-(1-hydroxyethyl)-3-hydroxypyridin-4-one; 1-ethyl-2-(1-hydroxyethyl)-3-hydroxypyridin-4-one; 1-hydroxypryidin-2one; 1-hydroxypryidin-2one; 2-Deoxy-2-(N-carbamoylmethyl-[N9-29-methyl-39-hydroxypyrid-49-one])-D-glucopyranose; 2-furoylcarboxaldehyde isonicotinoyl hydrazone (FIH); 2-hydroxy-1-naphthylaldehyde benzoyl hydrazone; 2-hydroxy-1-naphthylaldehyde isonicotinoyl hydrazone; 2-hydroxy-1-napthylaldehyde isonicotinoyl hydrazone; 2-methyl-3-hydroxy-4H-benzopyran-4-one (MCOH); 2-pyridylcarboxaldehyde 2-thiophenecarboxyl hydrazone (PCTH); 2-pyridylcarboxaldehyde benzoyl hydrazone (PCBH); 2-pyridylcarboxaldehyde isonicotinoyl hydrazone (PCIH); 2-pyridylcarboxaldehyde isonicotinoyl hydrazone (PCIH); 2-pyridylcarboxaldehyde m-bromobenzoyl hydrazone (PCBBH); 2-pyridylcarboxaldehyde m-bromobenzoyl hydrazone (PCBBH); 2-pyridylcarboxaldehyde p-aminobenzoyl hydrazone (PCAH); 2-pyridylcarboxaldehyde p-hydroxybenzoyl hydrazone (PCHH); 2-pyridylcarboxaldehyde thiophenecarboxyl hydrazone (PCTH); 311 2-hydroxy-1-napthylaldehyde isonicotinoyl hydrazone; 3-aminopyridine-2-carboxaldehyde thiosemicarbazone (Triapine®); 3-aminopyridine-2-carboxyaldehyde thiosemicarbazone; 3-hydroxypyridin-2-one; 3-hydroxypyridin-4-one; 4-[3,5-bis-(hydroxyphenyl)-1,2,4-triazol-1-yl]-benzoic acid (ICL670A), aminocarboxylates, BAPTA/AM (1,2-bis(2-aminophenoxy)ethane-N,N,N9,N9-tetraacetic acid acetoxymethyl ester); Catechols; CDTA cyclohexanediaminetetraacetic acid; cis-1,3,5-triaminocyclohexane; clioquinol; DDC diethyldithiocarbamate; Defarasirox; Deferiprone (1,2-dimethyl-3-hydroxypyrindin-4-one); Deferoxamine; Demercaptol; DFO desferrioxamine; DFOA; Diaminocyclohexane; diethylenetriaminepentaacetic acid; DMB; DMPA dimercaptopropionic acid; DMPS; DMSA dimercaptosuccinic acid; DPA (D-penicillamine); DTPA (diethylene triamine pentaacetic acid); EDTA, (ethylendiaminetetraacetic acid); Ferroportin-1; Hydroxamates; Hydroxycarboxylates; hydroxypyridinones; IDA iminodiacetic acid; MECAM; N,N_-bis-(2-hydroxybenzyl)ethylenediamine-N,N_-diacetic acid (HBED); N,N¢-bis(2-hydroxybenzyl)ethylenediamine-N,N¢-diacetic acid (HBED); N,N¢-Bis(3,4,5-trimethoxybenzyl)ethylenediamine-N,N¢-diacetic acid; N,N-dimethyl-2,3-dihydroxybenzoic acid; N4,NR,NR,N_,N_-pentakis[[((N-hydroxy-N-methyl]carbonyl)methyl]-2,6-diamino-4-azahexanoic hydrazide; NAPA N-acetyl-D-penicillamine; N-ethyl N,N,N-tris(pyridylmethyl)-cis,cis, 1,3,5,-triaminocyclohexane; NOTP (1,4,7-triazacyclononane-1,4,7-tris(methylenephosphonate)); NOTPME (1,4,7-triazacyclononane-1,4,7-tris(methylenephosphonatemonoethylester)); N-pyridine; NTA nitrilotriacetic acid; oxalic acid; pyridoxal hydrochloride; pyridoxal isonicotinoyl hydrazone; Pyridoxal isoonicotinoyl hydrazone (PIH); pyridoxal metachlorobenzoyl hydrazone; pyridoxal metafluorobenzoyl hydrazone; pyridoxal paramethoxybenzoyl hydrazone; rhizoferrin; salicylaldehyde benzoyl hydrazone; staphloferrin; staphloferrin; succinic acid; tachpyridine; TETA (triethylenetetraamine); tetraaza-4,7,10-tris(carboxymethyl)-1-cyclododecylacetylbenzylamine (DOTA-BA); TREN-(Me-3,2-hydroxipyridonate) (HOPO); TRENCAM; Triapine, 3-aminopyridine-2-carboxyaldehyde thiosemicarbazone; TTD tetratethythiuramdisulfide; TTHA triethylenetetraminehexaacetic acid; Deferiprone (1,2-dimethyl-3-hydroxypyridin-4-one); HYNIC (6-hydrazinonicotinamide); HYNIC-Kp-DPPB and HYNIC-Ko-DPPB where (HYNIC) 6-hydrazinonicotinamide, where K is lysine and DPPB is diphenylphosphine-benzoic acid; HPO 3-hydroxypyridin-4-one; 1-(2′-carboxyethyl)-2-methyl-3-hydroxypyridin-4-one; 1-(3′-hydroxypropyl)-2-methyl-3-hydroxypyridin-4-one; and 1-(2′-hydroxyethyl)-2-ethyl-3-hydroxypyridin-4-one.

20. The composition of claim 1 wherein the agent is a chelate between an imaging agent and a chelating agent.

21. The composition of claim 20 wherein the imaging agents are selected from the list consisting of ^{99m}-Tc-HYNIC, ^{99m}-Tc-DTPA, ^{99m}Tc-ciprofloxacin, ^{99m}Tc-methylene diphosphonate (MDP), indium or technetium-^{99m}hexamethylpropylene amine oxime (HMPAO), ¹¹¹In-DTPA-Folate, ^{99m}Tc diadenosine tetraphosphate (Ap4A; AppppA, P1,P4-di(adenosine-5*)-tetraphosphate) and its analog ^{99m}Tc AppCHClppA, ^{99m}Tc-HYNIC-IL-8., ^{99m}Tc-TRODAT-1, ^{99m}Tc-M-TRODAT, ^{99m}Tc-RP128, Gd(III) DOTA, ^{99m}Tc-HYNIC-folate, [^{99m}Tc(SG38)(tricine)(TPPTS)] (RP517), natCu-DOTADY1-TATE and DOTA-natI-DY1-TATE, ⁶¹Cu-DOTA-DY1-TATE, ^{99m}Tc labeled [(N-[2-((39-N9-propyl-[3,3,1]aza-bicyclononan-3a-yl)(20-methoxy-5-methyl-phenylcarbamate)(2-mercaptoethyl)amino)acetyl]-2-aminoethanethiolato]technetium(V) oxide), Albumin-(biotin)10-(gadopentetate)25, ^{99m}TcO(BAT-NI), (¹³¹1, ¹²³1, ′″In, (⁶⁷Ga, ⁶⁶Ga, ⁶⁴Cu, ⁶⁷Ga-deferoxamine-folate, ¹¹¹In-octadentate-DTPA-folate (g), ¹¹¹In-DTPA-NOON-pteroate., ^{99m}Tc(CO)3-DTPA-folate (g), ^{99m}Tc-EC20 (^{99m}Tc-Cys-Asp-Dap-D-Glu-Pte), ^{99m}Tc-oxa-PnAO-(a and g)-folate1155, ^{99m}Tc-ethylene dicysteine (^{99m}Tc-L,L-EC), Co-⁵⁶, Co-⁵⁷, Co-⁵⁸, and Co-⁶⁰-labelled vitamin B12, DCTA, and ¹¹¹Indium-DTPA-vitamin B12.

22. The composition of claim 1 wherein the agent is an anti-cancer chemotherapeutic agent.

23. The composition of claim 22, wherein the anti-cancer chemotherapeutic agent is selected from the list consisting of an antimetabolile, an antitumor, antibiotic, a mitotic inhibitor, a steroid, a sex hormone or hormone-like drug, an alkylating agent, platinum or other heavy metal, aq nitrogen mustard, a nitrosurea, a hormone agonist, a microtubule inhibitor a curcuminoid derivative and an inhibitor of deacetylase.

24. The composition of claim 1 wherein the agent is anthracyclin and one or both of 5-fluorouracil and/or chlorambucil.

25. The composition of claim 1 wherein the agent is a chemotherapeutic agent linked to a targeting agent.

26. The composition of claim 25 wherein the targeting agent is selected from the listing consisting of Amphiregulin, Antibodies including Avastin (bevacizumab), BEC2 (mitomomab), Tositumomab (Bexxar), Campath (alemtuzumab), CeaVab, herceptin (trastuzumab), IMC-C225 (centuximab), Lymphocide (epratuzumab), MDX-210, Mylotarg (Gemtuzumab), Panorex (Edorcolomab), Rituxan (Rituximab), Theragyn (pemtumomab), Zamyl, Zevalin (Ibritumomab), BAFF, BDNF, bFGF, BMP, BMP-4, BMP-7, CD209L, CSF, EGF, Fas-Ligand, Fc fragments, FGF, FGF2, FGFR1, FGFR4, Flt3, Flt3-Ligand, G-CSF, GM-CSF, hGH, hGHR, Growth Hormone Releasing Hormone and analogs, Growth Hormone Releasing Peptides, IFN, IgG, IGF-1, IGFBP-3, Interleukins, Langerin, LHRH analogs, LIF, L-Selectin, Lymphotoxin-a, MC-148, MCP-1, MIP-1a, MIP-1b, MLIF, NGF, NGFR, OSM, OX-40, PDGF, RANTES, Tf, TGF, TGF-b1, TNF, TNF-a, TPO, TrkA, TrkB, VEGF, Vitamin derivatives, including derivatives of vitamin B12, biotin, folate and/or riboflavin.

27. The composition of claim 1, wherein the agent is melanin.

28. The composition of claim 1, wherein the agent is an estrogen-like compound selected from the listing consisting of a chromene, an isoflavone and a coumestran.

29. The composition of claim 1, wherein the agent is a collagen fragment mimetic.

30. The composition of claim 1, wherein the agent is an anti-oxidant.

31. The composition of claim 1 wherein the agent is the listing consisting of DNA, siRNA, anti-sense RNA, effector RNA, and a microRNA.

32. The composition of claim 1 wherein the agent is selected from the listing consisting of fentanyl, oxycodone, codeine, dihydrocodeine, dihydrocodeinone enol acetate, morphine, desomorphine, apomorphine, diamorphine, pethidine, methadone, dextropropoxyphene, pentazocine, dextromoramide, oxymorphone, hydromorphone, dihydromorphine, noscapine, papverine, papveretum, alfentanil, buprenorphine, tramadol and opioid agonists and pharmaceutically acceptable salts, derivatives, homologs or analogs thereof.

33. The composition of claim 1 wherein the agent is a nitric oxide scavenger selected from the listing consisting of an inhibitor of NOS such as Niacinamide, vitamin B12 and derivatives, curcumin and derivatives L-NAME,N^{G}-nitro-L-arginine methyl ester; L-NMA, N^{W}-methyl-L-arginine, L-NMMA, N^{G}-monomethyl-L-arginine, S-Methylisothiourea sulphate, Aminoguanidine, 7-nitro indazole and N-nitro-L-arginine.

34. The composition of claim 1 wherein the agent is an inhibitor of cGMP specific phosphodiesterase type 5 (pDE5).

35. The composition of claim 1 wherein the agent is an nitric oxide synthase inhibitor selected from the listing consisting of Niacinamide, curcumin and derivatives, L-NAME,N^{G}-nitro-L-arginine methyl ester; L-NMA, N^{W}-methyl-L-arginine, L-NMMA, N^{G}-monomethyl-L-arginine, S-Methylisothiourea sulphate, Aminoguanidin, 7-nitro indazole and N-nitro-L-arginine.

36. The composition of claim 1 wherein the agent is selected from the listing consisting of curcumin, tetrahydrocurcumin, sodium circuminate, bisdemethoxycurcumin, demethoxycurcumin, 5′-methoxycurcumin and dihydrocurcumin, vitamin E, selenium, zinc, Vitamin C, azaleic acid, pantothenic acid, benzyl peroxide and extract of tea tree oil.

37. The composition of claim 1, wherein the microemulsion comprises an oil phase of 1 to 99% by weight; a water phase of 99 to 1% by weight; 1 to 99% by weight of surfactants or co-surfactants.

38. The composition of claim 1 wherein the oil phase is selected from the listing consisting of natural oils derived from plants or animals, such as vegetable oils, sunflower oils, coconut oils, almond oils; purified synthetic or natural di or triglycerides; phospholipids and their derivatives (such as lecithin or lysolecithin); fatty acid esters (such as isopropyl myristate, isopropyl palmitate, ethyl oleate, oleic acid ethyl ester); hydrocarbons (such as hexane, the n-decane through n-octadecane series); and/or glycerolysed fats and oils (such as glyceryl monooleate, glyceryl monocaprylate, glycerol monocaprate, propylene glycol monocaprylate, propyleme glycol monolaurate).

39. The composition of claim 1 wherein the oil phase is selected from the listing consisting of Labrafil M 1944 CS™, benzene, tetrahydrofuran, and n-methyl pyrrolidone, or halogenated hydrocarbons, such as methylene chloride, chloroform, Crodamol GTCC™ and Capmul MCM™.

40. The composition of claim 1 further comprising a surfactant selected from the listing consisting of an anionic surfactant, cationic surfactant and a non-ionic surfactant.

41. The composition of claim 2 wherein the agent is selected from the listing consisting of TNF superfamily (including TNF-alpha, TNFR1, TNFR2, BAFF, OX-40, Lymphotoxin-alpha, Fas-ligand); chemokines (including MCP-1, MIP-1a, MIP-1b, RANTES, IL-8 and viral like chemokine antagonist MC148); interleukins, interleukin receptors and antagonist (including IL-1a IL-1b, IL-2, IL-3, IL-4, IL-5, IL-6, IL-7, IL-8, IL-10, IL-11, IL-12, IL-13, IL-15, IL-16, IL-18, IL-20, IL-21, IL-22, IL-23, IL-24, IL-25, IL-26, IL-27, IL-28, IL-29, IL-30, their respective receptors including IL-1Ra, IL-2Ra, IL-2Rb, IL-2Rg, IL-3Ra, IL-4Ra, IL-5Ra, IL-6Ra, IL-7Ra, IL-10Ra, IL-11Ra, IL-13Ra, IL-15Ra as well as IL-1R Antagonist); the interferon family (including IFN-a2B, IFN-b1, IFN-g, IFN-y, IFN-aR2, IFN-aRa, IFN-aRb, IFN-gRa, IFN-gRb); lectins (including CD209 type I and II, E-Selectin, L-Selectin, P-Selectin, Langerin); growth factors and their receptors (including Amphiregulin, Angiopoietin, BDNF, beta-cellulin, BMPs (including BMP-2, BMP-4, BMP-7), CNTF, cripto, ECGF-1, EGF, EGFR, EPO, FGFs and their receptors (including FGF-1, FGF-2, FGF-5, FGF-7, FGF-9, FGF-11, FGF-12, FGF-13, FGF-14, FGF-14 FLAG, FGF-18, FGF-19, FGF-21, FGFR1, FGFR1, FGFR4, FGFR5), Flt3-Ligand and its receptor (including Flt3), G-CSF, GDNF, GM-CSF, GM-CSF-R, hGH and its receptor (including hGHR), IGF-1, IGFBP-3, M-CSF, Neuregulin, NGFs and its receptor (including NGF-b, NGFR), NT-3, PDGFs, TGFs and their receptors (including TGF-a, TGF-b, TGFbR2), Trk-A, Trk-B , TPO, VEGFs and their receptors (including VEGF-A, VEGF-B, VEGF-C, VEGF-D, VEGF-165, VEGFR); embryonic growth factors (such as Noggin, Nodal, SCF, Wnts, Wnt-2, Wnt-3, Wnt-3A, Wnt-4, Wnt-5A, Wnt-5A-FLAG-C, Wnt-5B, Wnt-6, Wnt-7A, Wnt-7B, Wnt-10A, Wnt-10B, Wnt-10B-FLAG-C, Wnt-11); adhesion molecules (such as adiponectin, ICAM), other cytokines and proteins, such as LIF, OSM, transferring and its receptor, hormones (such as insulin, calcitonin, adrenocorticotropin (ACTH), glucagon, somatostatin, somatotropin, thymosin, parathyroid hormone, pigmentary hormones, somatomedin, lutenizing hormone and agonists and antagonists thereof, chorionic gonadotropin, hypothalmic releasing factors, antidiuretic hormones, vasopressin, thyroid stimulating hormone, endorphins, enkephalins, biphalin and prolactin.); antibiotics (such as gentamycin, amikacin, neomycin, penicillin, streptomycin), enzymes (such as activin A asparaginase, adenosine deaminase, BACE-1, caspase-1, fucosyltransferase, furin, mTACE, sialyltransferase) Factor VIII, LH-RH analogs, anticoagulants such as Heparin, Warfarin, Herbal extracts such as those derived from Danshen, Devil's Claw, Eleuthero, Garlic, Ginger, Ginkgo, Horse Chestnut, Panax Ginseng, Papain, Red Clover, Saw Palmetto, capsaicin and vaccines (for instance, vaccines for Hepatitis ‘B’ surface antigen, typhoid and cholera vaccines) and plasminogen activator inhibitors, and small peptides such as MLIF (Met-Gln-Cys-Asn-Ser), and analogs thereof.

42. The composition of claim 1 wherein the agent is selected from the listing consisting of copper complexes of salicylates (including aspirin, choline magnesium trisalicylate, dilfunisal, salasalate, benorylate); copper complexes of phenylalkanoic acids (including carpofen, fenoprofen, fluribiprofen, ibuprofen, ketoprofen, naproxen, naproxen Na, suprofen, oxaprozin); copper complexes of acetic acids or indoles (including alclofenac, diclofenac, fenclofenac, indomthacin, sulindac, tolemetin); copper complexes of enolic acids (including isoxicam, meloxicam, piroxicam, tenoxicam); copper complexes of fenamic acids (including flufenamic acid, meclofenamate, mefenamic acid); copper complexes of napthylalkanones (including nabumetone); copper complexes of niflumic acid; copper complexes of pyranocarboxylic acids (including etodolan); copper complexes of pyrazolones (including phenylbutazone, oxyphenobutazone); copper complexes of pyrroles (including ketorolac); and copper complexes of COX-2 inhibitors (including celecoxib, parecoxib, valdecoxib, rofecoxib).

43. The composition of claim 1 wherein the agent is selected from the listing consisting of copper complexes of tyrosine, lysine, valproic acid, 2,2,-bypyridine, 1,10-phenanthroline, 2,9-dimethyl-1,10-phenanthroline, glycylglycine, cimetidine, sulfathiazole, tetraanhydroaminmobenzaldehyde, metronidazole and imidazole.

44. The composition of claim 1 wherein the agent is selected from the listing consisting of copper complexes with amino acids, aromatic carboxylic acid, corticoids, tetrazoles, histamines, penicillamines, anti-ulcer histamine antagonists, rantidine and cimetidine.

45. The composition of claim 1 wherein the agent is selected from the listing consisting of DMF, DMSO, Pyridine, Caffeine, Methylimidazole, Imidazole, 2-methylbenzimidazole, metronidazole, 2-methylimidazole, 3-picoline, 4-picoline, imidazole, 1-methylimidazole, diethylamine, nicotinamide, papaverine, N,N-dimethylacetamide, N-methyl-2-pyrrolidone, ethanol and methanol.

46. The composition of claim 1 wherein the agent is selected from the listing consisting of water soluble vitamins such as vitamin C, analogs or derivatives thereof; folic acid and analogs or derivatives thereof (including but not limited to methotrexate, aminopterin, 10-deazaminopterin, 10-ethyl-10-deazaaminopterin, 5,10-dideazatetrahydrofolate, folinic acid, 7-hydroxyaminopterin); niacin (nicotinic acid, vitamin B3) and analogs or derivatives thereof (including but not limited to beta-hydroxybutyrate, acipimox, niceritrol, nicotinamide (niacin)); thiamine (vitamin B-1), analogs and derivatives thereof; riboflavin (vitamin B2) and its analogs or derivatives thereof (including but not limited to 7-nor-7-chlororiboflavin, 8-nor-8-chlororiboflavin, 7-nor-7-bromoriboflavin, 8-nor-8-bromoriboflavin, 7-methylriboflavin, 8-methylriboflavin, 7,8-dimethylriboflavin, 7-nor-7-bromo-8-methylriboflavin, 7-methyl-8-nor-8-bromoriboflavin, 7-nor-7-chloro-8-methylriboflavin, 7-methyl-8-nor-8-chlororiboflavin, 8-nor-8-fluororiboflavin, 7-nor-7-chloro-8-nor-8-chlororiboflavin, 8-nor-8-aminoriboflavin, N(3)-methylriboflavin and 5-deaza-5-carbariboflavin); pyridoxine (vitamin B6), analogs or derivatives thereof; cyanocobalamin (vitamin B12), analogs or derivatives thereof, pantothenic acid (vitamin B5), analogs or derivatices thereof; biotin, analogs or derivatives thereof, and the vitamin E derivative Trolox. Suitable vitamins also include but are not limited to oil soluble vitamins such as vitamin A, analogs or derivatives thereof, such as acitretin (Soriatane); vitamin E (alpha tocopherol) analogs or derivatives thereof.

47. The composition of claim 1 wherein the agent is a vitamin B12 analog selected from the listing consisting of alpha (5,-6-dimethylbenzymidazolyl)-hydrogenobamide, hydroxocobalamin (OH—Cbl), methylcobalamin, adenosylcobalamin (AdeCbl), aquocobalamin, methylcobalamin, cyanocobalamin, carbanalide, 5methoxybenzylcyanocobalamin [(5-MeO)CN—Cbl], as well as the desdimethyl, monoethylamide and the methylamide analogs thereof including alkyl cobalamins in which the alkyl chain is linked to the corrin nucleus by a direct CoC covalent bond, chlorocobalamin, sulfitocobalamin, nitrocobalamin, thiocyanatocobalamin, benzimidazolecyanocobalamin derivatives such as 5,6dichlorobenzimidazole, 5-hydroxybenzimidazole, trimethylbenzimidazole, as well as adenosylcyanocobalamin [(Ade)CN—Cbl], cobalamin lactone, cobalamin lactam and the anilide, ethylamide, monocarboxylic and dicarboxylic acid derivatives of VB12 or its analogs, adeninylalkylcobalamin, adeninylethylcobalamin (AdeEtCbl), adeninylpropylcobalamin (AdePrCbl) and adeninylpentylcobalamin (AdePeCbl).

48. The composition of claim 1, wherein the composition or agent treats one or more dermatological conditions selected from dermatitis, bacterial, fungal, parasitic, viral infections of the skin, disorders of hair follicles and sebaceous glands, scaling papular diseases, inflammatory reactions, reaction to sunlight, bullous diseases, disorders of cornification, pressure sores, pigmentation disorders, disorders of sweating, benign and malignant tumors, muscle dystrophy and erectile dysfunction.

49. The composition of claim 48, wherein the agent has activity in promoting hair growth or inhibiting hair growth.

50. The composition of claim 48, wherein the agent has anti-infective activity, anti-inflammatory activity or anti tumor activity.

51. The composition of claim 48, wherein the agent has activity in skin whitening, skin repair or preventing or reducing skin damage.

52. Use of a composition of claim 1 in the manufacture of a medicament for the treatment of a condition.

53. Use of a microemulsion in the preparation of a transdermally deliverable medicament in the treatment of a disease condition in a subject.
